(19) United States
(12) Patent Application Publication
(10) Pub. No.: US 2015/0297761 A1
(43) Pub. Date: Oct. 22, 2015

(54) PEPTIDIC STRUCTURES INCORPORATING AN AMINO ACID METAL COMPLEX AND APPLICATIONS IN MAGNETIC RESONANCE IMAGING

(71) Applicant: THE GENERAL HOSPITAL CORPORATION, Boston, MA (US)

(72) Inventors: Peter Caravan, Cambridge, MA (US); Eszter Boros, Somerville, MA (US); Eric M. Gale, Charlestown, MA (US)

(21) Appl. No.: 14/441,892

(22) PCT Filed: Nov. 12, 2013
(86) PCT No.: PCT/US2013/069701

§ 371 Date: May 11, 2015

Related U.S. Application Data
(60) Provisional application No. 61/725,339, filed on Nov. 12, 2012.
(60) Provisional application No. 61/858,002, filed on Jul. 24, 2013.

(51) Int. Cl. A61K 51/08; C07K 5/062; C07F 5/00; C07K 5/02; A61K 49/10; A61K 49/14

(52) U.S. Cl. CPC A61K 51/088; A61K 49/108; A61K 49/14; C07F 5/00; C07K 5/02; C07K 5/06026; A61K 38/00

(57) ABSTRACT

A method for increasing the relaxivity of a contrast agent having a metal ion complexed to a chelator is disclosed. The metal ion complex is tethered to the remainder of the molecule by at least two points of attachment such that local motion is limited and higher relaxivity can be achieved. In one non-limiting example version of the invention, the alanine analogue of Gd(DOTA), Gd(DOTAla) wherein Gd is gadolinium and DOTA is 1,4,7,10-tetraazacyclododecane-1,4,7,10-tetraacetic acid was integrated into polypeptide structures. This resulted in very rigid attachment of the metal ion complex to the peptide backbone. Rigid molecular structures provide fewer degrees of rotational freedom, resulting in greater control over the rotational dynamics and resultant relaxivity. In the case of Gd(DOTAla), the metal complex is tethered to the peptide via the amino acid side chain to the DOTA moiety and via a dative bond from an amide oxygen to the Gd(III) ion.

### CROSS-REFERENCES TO RELATED APPLICATIONS

This application claims priority from U.S. Patent Application No. 61/725,339 filed Nov. 12, 2012 and U.S. Patent Application No. 61/858,002 filed Jul. 24, 2013.

### STATEMENT REGARDING FEDERALLY SPONSORED RESEARCH

This invention was made with government support under grant number EB 009062 awarded by the National Institute of Biomedical Imaging and Bioengineering. The government has certain rights in the invention.

### BACKGROUND OF THE INVENTION

1. Field of the Invention

The invention relates to contrast agents with high relaxivity for MRI scanners that are operated at higher magnetic fields. The invention also relates to methods for preparing the contrast agents.

2. Description of the Related Art

When a substance such as human tissue is subjected to a uniform magnetic field (polarizing field B₀), the individual magnetic moments of the excited nuclei in the tissue attempt to align with this polarizing field, but precess about it in random order at their characteristic Larmor frequency. If the substance, or tissue, is subjected to a magnetic field (excitation field B₁) that is in the x-y plane and that is near the Larmor frequency, the net aligned moment, Mz, may be rotated, or “tipped”, into the x-y plane to produce a net transverse magnetic moment Mt. A signal is emitted by the excited nuclei or “spins”, after the excitation signal B₁ is terminated, and this signal may be received and processed to form an image.

In magnetic resonance imaging (MRI) systems, the excited spins induce an oscillating sine wave signal in a receiving coil. The frequency of this signal is near the Larmor frequency, and its initial amplitude, is determined by the magnitude of the transverse magnetic moment Mt. The amplitude, A, of the emitted NMR signal decays in an exponential fashion with time, t. The decay constant 1/T*₂ depends on the homogeneity of the magnetic field and on T₂, which is referred to as the “spin-spin relaxation” constant, or the “transverse relaxation” constant. The T₂ constant is inversely proportional to the exponential rate at which the aligned precession of the spins would dephase after removal of the excitation signal B₁ in a perfectly homogeneous field. The practical value of the T₂ constant is that tissues have different T₂ values and this can be exploited as a means of enhancing the contrast between such tissues.

Another important factor that contributes to the amplitude A of the NMR signal is referred to as the spin-lattice relaxation process that is characterized by the time constant T₁. It describes the recovery of the net magnetic moment M to its equilibrium value along the axis of magnetic polarization (z). T₂ relaxation is associated with a decrease in spin coherence, and T₁ relaxation occurs due to a paramagnetic shift at the probe site and subsequent exchange of bound protons with the surrounding bulk water. The T₁ time constant is longer than T₂, much longer in most substances of medical interest. As with the T₂ constant, the difference in T₁ between tissues can be exploited to provide image contrast.

The reciprocals of these relaxation time constants are termed relaxation rates and denoted R₁ and R₂ where R₁=1/T₁ and R₂=1/T₂.

Contrast agents are exogenous molecules or materials that can alter the relaxation properties of tissue and induce image contrast. Contrast agents are typically paramagnetic, superparamagnetic, or ferromagnetic materials. Contrast agents are also sometimes referred to as imaging probes.

The extent to which a given contrast agent can alter the relaxation rate is termed relaxivity. Relaxivity is defined as the difference in the relaxation rate of a sample measured with and without the contrast agent. This relaxation rate difference is then normalized to the concentration of the contrast agent. Relaxivity is expressed as a lowercase “r” with a subscript “1” or “2” which refers to either the longitudinal or transverse relaxivity respectively. For instance longitudinal relaxivity, r₁, is defined as r₁=(R₁−R₁⁰)/C where R₁ is the relaxation rate in s⁻¹ measured in the presence of the contrast agent, R₁⁰ is the relaxation rate in s⁻¹ measured in the absence of contrast agent, and C is the concentration in mM of the contrast agent. Relaxivity has units of mM⁻¹s⁻¹. For contrast agents that contain more than one metal ion, relaxivity can be expressed in terms of the metal ion concentration (‘per ion’ or ‘ionic relaxivity’) or in terms of the molecular concentration (‘per molecule’ or ‘molecular relaxivity’). Relaxivity is an inherent property of contrast agents.

In an effort to elicit clinically-desired contrasts, MRI contrast agents have been developed that are designed to affect the relaxation periods. Not surprisingly, there are contrast agents that are used clinically to adjust T₁ contrast and those that are used clinically to adjust T₂ contrast.

T₁-weighted (T₁w) imaging provides image contrast where tissues or regions of the image are bright (increased signal intensity) when the T₁ of water in that region is short. One way to increase image contrast is to administer a paramagnetic complex or material based on gadolinium (Gd), manganese, or iron. For example, clinically utilized Gd(III) imaging probes comprise nonacoordinate, ternary complexes where the Gd(III) ion is held within an octadentate polyaminocarboxylate ligand and coordinated by one rapidly exchanging water ligand. Due to strong paramagnetism (S=7/2) and slow electronic relaxation (T1e), complexes of Gd(III) are ideal for generating T₁ contrast (example 5, ref 1). This paramagnetic contrast agent shortens the T₁ of water molecules that it encounters and results in positive image contrast. The degree to which a given concentration of contrast agent can change T₁ is the relaxivity as noted above. Compounds that have higher relaxivity provide greater T₁w signal enhancement than compounds with low relaxivity; alternatively a high relaxivity compound can provide equivalent signal enhancement to a low relaxivity compound but at a lower concentration than the low relaxivity compound. Thus, high relaxivity compounds are desirable because they enable greater enhancement of lesions and improve diagnostic confidence; alternately, they can be used at lower doses and thus improve the safety margin of the contrast agent.

An increasing number of MRI scanners being sold today operate at higher magnetic fields, e.g., at 3 Tesla or 7 Tesla. The signal:noise ratio (SNR) increases with increasing magnetic field. In addition, the inherent T₁ of tissue is also decreasing with increasing field and thus the sensitivity of Gd-based contrast agents should increase with field if probe relaxivity (r₁) is field independent. Unfortunately for many contrast agents, r₁ typically decreases with field faster than the decrease in baseline T₁. However, by controlling the rotational dynamics of the contrast agent, it is possible to create high relaxivity contrast agents that exhibit high relaxivity at high fields.

To optimize relaxivity, the contrast agent should undergo rotational diffusion with a rate close to the Larmor frequency of hydrogen at the applied magnetic field, e.g., ˜127 MHz at 3 Tesla. In general, there are three modular parameters readily available to the chemist: the rate of inner-sphere water exchange (kₑₓ=1/τₘ; τₘ=mean residency time of the H₂O ligand), the rotational correlation time of the complex (τ_{R}) and the hydration number of the Gd(III) ion (q). The property τₘ is dictated by the ligand frame and choice of donor groups, and the manifestations of commonly used functional groups on τₘ have been explored in detail (example 5, refs 4, 5). The optimal range of τₘ values depends on both τ_{R} and magnetic field, (example 5, refs 5, 7) however 10<τₘ<30 ns is optimal across all field strengths. Increasing τ_{R} can afford tremendous r₁ enhancement at relatively low field strength (<1.5 T) (example 5, ref 6). This strategy has met with much success by either multimerization (example 5, ref 8) or through covalent or non-covalent conjugation to macromolecular entities (example 5, refs 9-11). In order to design molecules with precisely tuned dynamics it is desirable to have the paramagnetic ion rotate isotropically along with the entire molecule. For larger molecules, the metal ion complex may be tethered to another part of the molecule via a flexible linker. This flexible linker results in local rotational motion that is faster than the overall rotational diffusion of the entire molecule. Fast local motion limits relaxivity.

Alternatively, modulation of the hydration number q can significantly affect the r₁ of contrast agents including Gd(III) complexes, and r₁ tends to scale with this parameter independent of field. However, an increase in q requires a reduction in available ligand donors and can come at the cost of reduced thermodynamic stability and kinetic inertness with respect to transchelation of the Gd(III) ion. Therefore, one must be judicious in ligand design. A handful of q>1 Gd(III) complexes featuring hexa- and heptadentate ligands have been prepared and evaluated, (example 5, refs 12-16) with notable examples highlighted in FIG. 26. These ligands are highly pre-organized and are designed for maximal stability given the reduction in ligand denticity (<8). In light of this, probes displaying enhanced relaxivity represent desirable and oft sought targets in molecular imaging.

Therefore, there is a need for higher relaxivity contrast agents for magnetic resonance imaging systems being operated at higher magnetic fields. There is also a need for methods for preparing such higher relaxivity MRI contrast agents.

### SUMMARY OF THE INVENTION

The invention meets the foregoing needs by providing a method of increasing the relaxivity of a contrast agent having a metal ion complexed to a chelator. By tethering the metal ion complex to the remainder of the molecule by at least two points of attachment, local motion is limited and higher relaxivity can be achieved.

In one non-limiting example version of the invention, we investigated applications of the alanine analogue of Gd(DOTA), Gd(DOTAla), wherein Gd is gadolinium and DOTA is 1,4,7,10-tetraazacyclododecane-1,4,7,10-tetraacetic acid. Fmoc (fluorenylmethyloxycarbonyl) protected DOTAla suitable for solid phase peptide synthesis was synthesized from cyclen and a mesylated serine derivative in five steps and 15% over all yield. The integration of this unnatural amino acid into polypeptide structures followed by complexation with Gd(III) results in very rigid attachment of the metal ion complex to the peptide backbone. Rigid molecular structures provide fewer degrees of rotational freedom, resulting in greater control over the rotational dynamics and resultant relaxivity. In the case of Gd(DOTAla), the metal ion complex is tethered to the peptide via the amino acid side chain to the DOTA moiety and via a dative bond from an amide oxygen to the Gd(III) ion.

We also investigated the application of Gd(DOTAla) as a blood pool imaging agent, by targeting human serum albumin (HSA). We took advantage of the possibility to attach multiple functional groups capable of targeting HSA. As the system is modular, rapid synthesis of a variety of derivatives is possible and allows for accelerated screening for the optimal contrast agent. We have also prepared multimeric peptide based contrast agents of defined size and relaxivity. These peptide based contrast agents have the general synthetic flexibility of peptides and can also incorporate other imaging reporters (e.g. fluorescent moieties or positron emitters) or specific targeting vectors.

We provide a synthetic intermediate which allows for efficient multimerization or integration of the chelate DOTAla into polypeptides using solid phase peptide synthesis. We have also found the corresponding Gd complexes to have excellent properties for development of molecular imaging agents for MRI. The modular nature of the invention allows for facile synthesis of multimodal imaging probes, targeted imaging probes, or theranostics. Furthermore, the well-defined structure of (DOTAla) enables rational peptide or protein design. Other paramagnetic metals may be incorporated that provide relaxation or chemical shift effects that can be used for structural elucidation of proteins.

Furthermore, we have designed and evaluated a new ligand framework, CyPic3A, capable of forming ternary complexes with Gd(III) featuring two coordinated waters. The two bound waters afford favorable r₁ and appear to be impervious to displacement by endogenously encountered bidentate ions such as phosphate, carbonate and lactate. Despite the heptadentate nature of the ligand, CyPic3A forms Gd(III) complexes with thermodyanamic stability and kinetic inertness comparable to FDA approved probes employing octadenate ligands. CyPic3A holds promise regarding the development of highly efficient imaging probes amenable to use across a wide range of magnetic fields.

These and other features, aspects, and advantages of the present invention will become better understood upon consideration of the following detailed description, drawings, and appended claims.

### BRIEF DESCRIPTION OF THE DRAWINGS

FIG. 1 shows the factors that influence the relaxivity of Gd complexes.

FIG. 2 shows the influence of τ_{R} and τ_{M} on relaxivity at different field strengths.

FIG. 3 shows the disclosed method for optimizing τ_{R} and τ_{M} for Gd-based high field imaging.

FIG. 4 shows the monomeric complex Gd(L1). As seen from the different single “modules” implemented, the peptide-based synthesis is versatile.

FIG. 5 shows a schematic depiction of all complexes synthesized and evaluated in Example 1.

FIG. 6 shows T₁ weighted images acquired at 1.5 T, at 25° C.

FIG. 7 shows the molecular parameters that influence relaxivity: rotation (τ_{R}), water exchange (τ_{M}), hydration number (q), and electronic relaxation (T₁ₑ).

FIG. 8 shows in the top row: a schematic depiction of previously explored Gd complexes with τ_{R} between 0.35 and 1 ns (A, B, C), as well as the novel approach described herein (Example 2, D). In the bottom row: examples for molecules reported using approaches A,¹⁷ B⁵ and C¹⁸, see Example 2.

FIG. 9 shows at the top: structures of approved Gd-based agents [Gd(DOTA)(H₂O)]⁻ and [Gd(HP-DO3A)(H₂O)]. At the bottom: various approaches to conjugated DOTA derivates. E and F show previously investigated Gd(DOTA) type complexes with optimal water exchange properties. G represents a previously explored lysine derivative of DOTA without the dual attachment strategy. Compound H represents our approach of Example 2 using dual attachment to the peptide to limit internal motion of the complex.

FIG. 10 shows chemical synthetic Scheme 1, for the synthesis of compound 6 of Example 2.

FIG. 11 shows synthetic Scheme 2, for the synthesis of new contrast agents described in Example 2. (i) 1.) 4 eq. Fmoc-X (X=Gly, Phe, Cys(Acm)), 4 eq. HATU, 4.5 eq. NMM, DMF, 12 hours, 2.) 20% piperidine in DMF, 2 hours; (ii) 1.) 1.5 eq. 6, 2 eq. HATU, 3 eq. NMM, 2.) 20% piperidine in DMF (iii) 10 eq. I₂, DMF, 6 hours, (iv) TFA:DDT:TIPS:Water (9.5:0.25:0.25:0.25), 6 hours, (v) DMSO (2% v/v), H₂O (pH 8), 12 hours.

FIG. 12 shows the temperature dependence of the ¹⁷O NMR (11.7 T) reduced transverse relaxation rates of GdL1 (6.8 mM). The solid line represents fit to the data to determine the water exchange rate.

FIG. 13 shows the kinetic inertness of Gd(DOTAla) derivatives. Transchelation of Gd from linear complexes GdL1(), Gd₂L2(▪), Gd₃L3 (▴) and [Gd(HP-DO3A)(H₂O)] (♦) to MS-325 at pH 3, 37° C. Data shown is for the first 168 hours of the reaction.

FIGS. 14A and 14B show relaxivities of Gd₃L3(), MS-325 with excess HSA (▾) and [Gd(HP-DO3A)(H₂O)] (♦) as a function of magnetic field at 37° C. (A) Relaxivity plotted per [molecule] showing that Gd₃L3 with its intermediate correlation time is a much more potent relaxation agent that slow or fast tumbling compounds at 60 MHz and higher frequencies. (B) Relaxivity plotted per [Gd] shows that the intermediate correlation time of Gd₃L3 results in higher relaxivities at high fields.

FIG. 15 shows a gradient echo MR image acquired at 4.7 T (T_{E}=6 ms and T_{R}=30 ms, flip angle=90°) of equimolar solutions of [Gd(HP-DO3A)(H₂O)], MS-325 in HSA (0.66 mM), Gd₃L3 in H₂O at both equal Gd(III) ionic concentration and equal molecular concentration.

FIG. 16 shows measurement of relaxivity in dependence of disulfide bond reduction. Solid bars represent cyclic, shaded bars represent TCEP reduced values.

FIG. 17 shows kinetic inertness of linear complexes GdL1(), Gd₂L2(▪), Gd₃L3 (▴) and Gd(DO3A-HP) (♦) within 360 hours (left) and GdL1(), Gd(DTPA) (◯) and Gd(DO3A-HP) (♦) within first 30 hours (right).

FIG. 18 shows a T₁ weighted image acquired at 1.5 T, at 25° C. All complexes are at 0.1±0.014 mM/Gd concentration. Gd(DO3A-HP) and HEPES buffer are shown as a reference. TR: 5.2 ms, TE: 50 ms, flip angle: 60°.

FIG. 19 shows the synthetic scheme for compounds 2-7 of Example 3.

FIG. 20 shows the synthetic scheme for compounds 8-14 of Example 3.

FIG. 21 shows the synthetic scheme for compounds 15-19 of Example 3.

FIG. 21A shows a general synthetic scheme for compounds of Example 3, including without limitation Gd(8) and Gd(9).

FIG. 22 shows a summary of the synthetic scheme for preparing an example compound of the invention.

FIG. 23 shows the limited rotational freedom between the peptide backbone and chelate complex of an example compound of the invention wherein the peptide backbone amide coordinates to Gd, providing additional attachment and decrease of local motion.

FIG. 24 shows a summary of the synthetic scheme for preparing an example multimeric compound of the invention.

FIG. 25 shows trifunctional derivatives based on an example compound of the invention for human serum albumin binding.

FIG. 26 shows previously studied Gd(III) complexes of hydration state q=2 and [Gd(CyPic3A)(H₂O)₂]⁻, bottom right.

FIG. 27 shows an exemplary chelate compound and a metal-chelate complex of the present invention.

FIG. 28 shows a scheme for the synthesis of CyPic3A.

FIG. 29 shows a number of bifunctional analogs of intermediate compounds used in the synthesis of CyPic3A.

FIG. 30 shows relaxivity values recorded for [Gd(CyPic3A)(H₂O)₂]⁻ at pH 7.4, 310 K in different media. The line corresponds to the measured r₁ of [Gd(DTPA)(H₂O)]²⁻ (3.26 mM⁻¹s⁻¹) in 50 mM HEPES.

FIG. 31 shows values for the equilibrium (K_{comp}) constant of [Gd(DTPA)(H₂O)]²⁻ upon challenge with CyPic3A and calculated K_{comp} for challenge with select FDA approved probes (HP-DO3A, DTPA-BMA) and previously reported heptadentate chelators (AAZTA, HOPO, DO3A, PCTA).

FIG. 32A shows liquid chromatography (LC) data for CyPic3A at 280 nm detection (lower trace) and mass spectrometry (MS) chromatogram of extracted m/z=424.2 [MW+H⁺] (upper trace).

FIG. 32B shows liquid chromatography (LC) data for [Gd(CyPic3A)(H₂O)₂]⁻ at 280 nm detection (lower trace) and mass spectrometry (MS) chromatogram of extracted m/z=579.0 [MW+2H⁺] (upper trace).

FIG. 32C shows liquid chromatography (LC) of [Eu(CyPic3A)(H₂O)₂]⁻ at 280 nm detection (lower trace) and mass spectrometry (MS)chromatogram of extracted m/z=574.0 [MW+2H⁺] (upper trace). The species eluting at 2.48 min (*) is CyPic3A, added in excess to ensure full chelation of Eu(III) during the luminescence lifetime measurements.

FIG. 33 shows Gd(III) chelators that CyPic3A was compared against. DTPA, MS-325-L DTPA-BMA and HP-DO3A are the ligand components of the clinically utilized Magnevist®, Ablavar® (MS-325), Omniscan® and ProHance®; each of which forms a ternary Gd(III) complex of q=1.

FIG. 34 shows time-dependence on luminescence intensity of [Eu(CyPic)(H₂O)₂]⁻ in D₂O (left) and H₂O (right); monoexponential fits are in black. Note the different scale for the faster decaying sample in H₂O.

FIG. 35 shows the relative change in r₁ of 1 mM [Gd(CyPic3A)(H₂O)₂]⁻ in pH 7.4 HEPES buffer (50 mM) as a function of carbonate (grey-top circles) and L-lactate (black) concentration.

FIG. 36 shows a time profile of [Gd(CyPic3A)(H₂O)₂]⁻ (0.53 mM at t=0) conversion to MS-325 during challenge with 1 mol-equiv. MS-325-L in 25 mM pH 7.4 Tris buffer.

FIG. 37A shows liquid chromatography (LC) traces of [Gd(CyPic3A)(H₂O)₂]⁻ vs. 1 mol-equiv. MS-325-L at 220 nm, where MS-325-L (7.88 min) and MS-325 (8.21 min) are most easily differentiated. The data was acquired at 25 days.

FIG. 37B shows liquid chromatography (LC) traces of [Gd(CyPic3A)(H₂O)₂]⁻ vs. 1 mol-equiv. MS-325-L at 280 nm, where CyPic3A (2.48 min) and [Gd(CyPic3A)]⁻ (6.90 min) are most easily differentiated. The data was acquired at 25 days.

FIG. 38 shows a time profile of transmetallation of [Gd(CyPic3A)(H₂O)₂]⁻ (middle trace), Gd(DTPA-BMA)(H₂O) (bottom trace) and [Gd(DTPA)(H₂O)]²⁻ (top trace) with 1 equiv. Zn(II). The reaction is monitored by following 1/T₁ with time, as the liberated Gd(III) precipitates as Gd₂(PO₄)₃ and does not contribute to T₁. (2.5 mM Gd(III)-complex, 2.5 mM Zn(OTf)₂, pH 7 phosphate buffer (50 mM), 310 K).

FIG. 39 is a block diagram of an example magnetic resonance imaging (MRI) system for use with a compound of the present invention.

FIG. 40 shows a synthesis scheme for all DOTAlaP-derivatives of Example 6.

FIG. 41 shows the temperature dependence of the ¹⁷O NMR (11.7 T) reduced transverse relaxation rates of 3 of Example 6 (6.73 mM, left) and 5a of Example 6 in PBS (4.16 mM, right). The solid line represents fit to the data to determine the mean water residency time τ_{M}.

FIG. 42 shows at the left: T1 weighted images of U87 brain tumors enhanced by either Gadovist or Gd₃L3-COOH, and at the right: Quantification of CNR achieved with either Gadovist or Gd₃L3-COOH.

FIG. 43 shows 1 minute post injection images obtained with MS-325 (left) and Gd(4a). Gd(4a) of Example 6 shows visibly better contrast in the vena cava, which can be quantified as 38±2% better contrast (vs. muscle). The same dose of agent was used for both scans.

FIG. 44 shows from far left to right: A schematic overview of imaged area on a mouse, coronal slices are shown on top, axial slices on bottom. Pre-injection T₁ weighted scan (t=0 minutes) followed by continuous acquisition of T₁ weighted scans (t=2 minutes, 25 minutes) with same parameters as the pre-injection scan. Early time point (2 minutes) shows enhancement of vasculature. The late time point (25 minutes) shows enhancement of hepatic tissue while the agent has entirely cleared from the blood pool.

### DETAILED DESCRIPTION OF THE INVENTION

The invention provides a compound for diagnosing or treating a subject. In one preferred embodiment of the invention, the compound is used as a contrast agent in a diagnostic imaging technique such as magnetic resonance imaging (MRI), positron emission tomography (PET), and/or single-photon emission computed tomography (SPECT). For purposes of the present invention, “treating” or “treatment” describes the management and care of a patient for the purpose of combating a disease, condition, or disorder. The terms embrace both preventative, i.e., prophylactic, and palliative treatment. Treating includes the administration of a compound of the present invention to prevent the onset of the symptoms or complications, alleviating the symptoms or complications, or eliminating the disease, condition, or disorder. A “subject” is a mammal, preferably a human.

The form in which the compound is administered to the subject is not critical. For example, the compound of the invention can be administered directly to tissue being diagnostically imaged or treated, to a body fluid that contacts the tissue, or to a body location from which the compound can diffuse or be transported to the tissue being diagnostically imaged or treated.

The compound can be administered alone or as part of a pharmaceutically acceptable composition. The relative amounts of the compound of the invention, a pharmaceutically acceptable carrier, and any additional active ingredients in a pharmaceutical composition of the invention will vary, depending upon the identity, size, and condition of the human treated and further depending upon the route by which the compound is to be administered. Other pharmaceutically active compounds can be selected to treat the same disease as a compound of the invention or a different disease. A compound of the invention, optionally comprising other pharmaceutically active compounds, can be administered to a subject parenterally, for example, intravenously, intramuscularly, subcutaneously, intracerebrally or intrathecally.

In one non-limiting example embodiment of the invention, the compound for diagnosing or treating a subject has the formula (I):

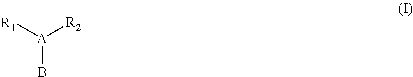

wherein A is a first amino acid residue, and B is a chelate complex comprising a chelator and a metal ion, the chelator comprising a ring of atoms. The chelator forms at least one coordinate bond with the metal ion, and the first amino acid residue is bonded to an atom of the ring of the chelator. The first amino acid residue has a carbonyl group oxygen that forms a coordinate bond with the metal ion. R₁ is a moiety comprising hydrogen, or an amino acid residue, or combinations thereof; and R₂ is a moiety comprising hydrogen, or an amino acid residue, or combinations thereof. At least one of R₁ and R₂ comprises an amino acid residue. By “amino acid residue”, we mean an amino acid that has a hydrogen ion removed from the amine end, or a hydrogen ion or hydroxyl ion removed from the carboxyl end, or both.

The first amino acid residue can be selected from residues of alanine, cysteine, aspartic acid, glutamic acid, phenylalanine, glycine, histidine, isoleucine, lysine, leucine, methionine, asparagine, pyrrolysine, proline, glutamine, arginine, serine, threonine, selenocysteine, valine, tryptophan, and tyrosine. In non-limiting example versions of the compound, the peptide backbone (R₁ and A and R₂) can include two or more residues such as residues of alanine, cysteine, phenylalanine, glycine, and tyrosine. In another non-limiting example, R₁ includes a cysteine residue, and R₂ includes a cysteine residue. Optionally, the cysteine residues in R₁ and R₂ can be linked by a disulfide bond. Preferably, the first amino acid residue is an alanine residue.

The peptide backbone can include any number of residues; however, for ease of synthesis and reproducibility in clinical trials, it may be preferred to limit the residues in the peptide to 20 or less, and more preferably, 10 or less. The peptide backbone can be attached to pharmacologically active groups, immunoreactive haptens, polymers, nanoparticles, proteins, other peptides, enzymes, drugs, and vitamins. In one example form, the peptide backbone is attached to a protein, enzyme, peptide, antibody, or drug that can target a specific site (e.g., tumor) in a subject (human or animal) undergoing a diagnostic medical procedure. For example, at least one of R₁ and R₂ can comprise a blood plasma binding moiety, or at least one of R₁ and R₂ can comprise a targeting moiety that can target a site in the subject.

In another non-limiting example embodiment of the invention, the compound for diagnosing or treating a subject has the formula (II):

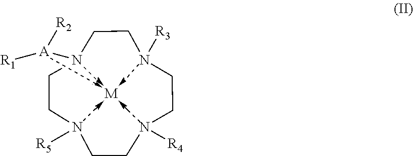

wherein A, R₁, and R₂ are as defined above, and at least one of R₁ and R₂ comprises an amino acid residue. R₃, R₄, and R₅ can be independently selected from the group consisting of H, CH₂CO₂H, CH₂CH₂CO₂H, CH₂C(O)NR⁶R⁷, CH₂NHCOR⁶, CH₂C(O)N(OH)R⁶, CH₂C(O)NHSO₂R⁶, CH₂NHSO₂R⁶, CH₂N(OH)C(O)R⁶, CH₂P(R⁶)O₂R⁷, CH₂PO₃R⁶R⁷, wherein R⁶ and R⁷ are independently selected from the group consisting of H, CO₂H, C₁-C₆ alkyl, C₁₋₆CO₂H, CH(CO₂H)C₁₋₆CO₂H, C₁₋₆CF₃, C₁₋₆CCl₃, C₁₋₆CBr₃, C₁₋₆Cl₃, or C₁₋₆PO₃R⁹R¹⁰, wherein R⁹ and R¹⁰ are independently selected from the group consisting of H, CO₂H, C₁-C₆ alkyl, C₁₋₆CO₂H, CH(CO₂H)C₁₋₆CO₂H. M is a metal ion, and an atom of at least one of R₃, R₄, and R₅ in the compound forms a coordinate bond with the metal ion.

In one non-limiting preferred version of the invention, the compound has the formula (III):

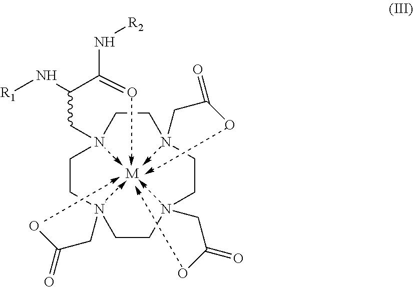

wherein R¹ and R² are as defined above, and at least one of R₁ and R₂ comprises an amino acid residue, and M is a metal ion, preferably Gd³⁺. In formula (III), group A of the compounds of Formulas (I) and (II) is alanine.

Compounds according to the invention can also synthesized to have a multimeric structure. An example multimeric compound for diagnosing or treating a subject has the formula (IV):

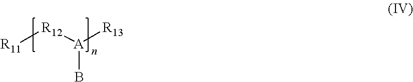

wherein A is a first amino acid residue, and B is a chelate complex comprising a chelator and a metal ion wherein the chelator comprises a ring of atoms. The chelator forms at least one coordinate bond with the metal ion, and the first amino acid residue is bonded to an atom of the ring of the chelator. The first amino acid residue has a carbonyl group oxygen that forms a coordinate bond with the metal ion. R₁₁ is a moiety comprising hydrogen, or an amino acid residue, or combinations thereof; R₁₂ is nothing or a moiety comprising hydrogen, or an amino acid residue, or combinations thereof, and R₁₃ is a moiety comprising hydrogen, or an amino acid residue, or combinations thereof. At least one of R₁₁ and R₁₃ comprises an amino acid residue, and n is an integer of 2 or more.

In the compound of formula (IV),

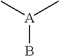

can have the formula (V)

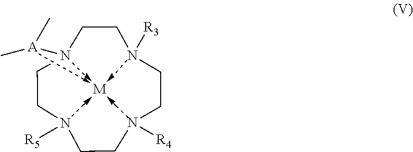

wherein A, R₁, and R₂ are as defined above, and at least one of R₁ and R₂ comprises an amino acid residue. R₃, R₄, and R₅ can be independently selected from the group consisting of H, CH₂CO₂H, CH₂CH₂CO₂H, CH₂C(O)NR⁶R⁷, CH₂NHCOR⁶, CH₂C(O)N(OH)R⁶, CH₂C(O)NHSO₂R⁶, CH₂NHSO₂R⁶, CH₂N(OH)C(O)R⁶, CH₂P(R⁶)O₂R⁷, CH₂PO₃R⁶R⁷, wherein R⁶ and R⁷ are independently selected from the group consisting of H, CO₂H, C₁-C₆ alkyl, C₁₋₆CO₂H, CH(CO₂H)C₁₋₆CO₂H, C₁₋₆CF₃, C₁₋₆CCl₃, C₁₋₆CBr₃, C₁₋₆Cl₃, or C₁₋₆PO₃R⁹R¹⁰, wherein R⁹ and R¹⁰ are independently selected from the group consisting of H, CO₂H, C₁-C₆ alkyl, C₁₋₆CO₂H, CH(CO₂H)C₁₋₆CO₂H. M is a metal ion, and an atom of at least one of R₃, R₄, and R₅ in the compound forms a coordinate bond with the metal ion.

In the compound of formula (IV),

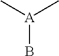

can have the formula (VI)

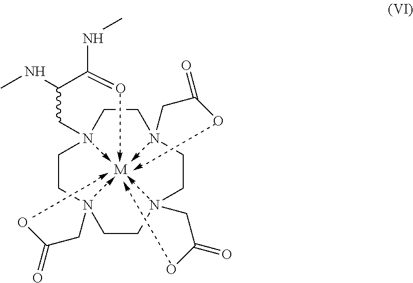

wherein M is the metal ion.

Group A in the compounds of Formulas (I), (II), and (IV) has limited rotational freedom with respect to group B due to very rigid attachment of the metal ion complex to the peptide backbone. Rigid molecular structures provide fewer degrees of rotational freedom, resulting in greater control over the rotational dynamics and resultant relaxivity. In the case of the compounds of Formulas (I) and (II) and (IV), the metal ion complex is tethered to the peptide via the amino acid (e.g., alanine) residue side chain to the chelator (e.g., DOTA) moiety and via a coordinate bond from an amide oxygen to the metal ion (e.g., Gd³⁺).

In the compounds of formulas (I) and (II) and (IV), the metal ion can be selected from ions of gadolinium, europium, terbium, manganese, iron, ⁴⁵Ti, ⁵¹Mn, ⁵²Mn, ^{52m}Mn, ⁵²Fe, ⁶⁰Cu, ⁶¹Cu, ⁶²Cu, ⁶⁴Cu, ⁶⁷Cu, ⁶⁷Ga, ⁶⁸Ga, ⁷²As, ⁸⁶Y, ⁸⁹Zr, ⁹⁰Nb, ^{94m}Tc, ^{99m}Tc, ¹¹⁰In, ¹¹¹In, ¹¹³In, ¹⁷⁷Lu, ²⁰¹Tl, ²¹²Pb ²¹³Bi, or ²²⁵Ac. The metal ion can be paramagnetic. The metal ion can be selected from paramagnetic metal ions having atomic numbers 21-29, 43, 44, and 57-83. The chelated metal ion enables the compound to be quantified by magnetic resonance imaging (MRI), positron emission tomography (PET), and/or single-photon emission computed tomography (SPECT). For MRI, the metal ion can be, for example, a gadolinium ion (Gd³⁺) or a manganese ion (Mn²⁺). For PET detection, the metal ion can be a positron-emitting radionuclide (e.g., ⁶⁴Cu, ⁶⁸Ga) which will annihilate to form two gamma rays which will be detected by the PET camera. For SPECT detection, the chosen metal ion (e.g., ions of ¹¹¹In) should produce a large number of photons. Alternatively, a non-metal, non-chelated positron-emitting radionuclide (e.g., ¹⁸F, ¹¹C, ¹³N, ¹⁵O, ⁷⁵Br, ⁷⁶Br, ⁸²Rb, ¹²⁴I) can replace an atom of the compound for PET detection.

In the compounds of formulas (I) and (II) and (III), at least one of R¹ and R² can comprise a fluorescent moiety. In the compounds of formula (IV), at least one of R¹¹ and R¹³ can comprise a fluorescent moiety. Fluorescent compounds can be used in molecular imaging both in vitro and in vivo. For in vivo imaging, near infrared (NIR) fluorophores have ideal absorption/emission wavelengths between 550 and 1000 nanometers, which minimize autofluorescence interference from tissue and have minimal overlap with biological chromophores such as hemoglobin. By including an NIR fluorophore in any of the compounds of Formulas (I) and (II) and (III) and (IV), the compound can be successfully applied to in vivo imaging of tissue (such as a tumor). Preferably, the fluorescent moiety has an absorption wavelength maxima in the range of 650 to 850 nanometers for imaging of tissue. The fluorescent moiety can be selected from cyanine dyes, carbocyanine dyes, and CyAL dyes, such as the carbocyanine dyes described in United States Patent Application Publication No. 2011/0286933. In such an imaging method, the compound including an NIR fluorophore is administered to a region of interest of a subject, light is directed into the subject, fluorescent light emitted from the subject is detected, and the detected light is processed to provide an image that corresponds to the region of interest of the subject.

The compounds of Formulas (I) and (II) and (III) and (IV) can be advantageous when used as a contrast agent in MRI, particularly as a higher relaxivity contrast agent for magnetic resonance imaging systems being operated at higher magnetic fields. The compounds of Formulas (I), (II), (III) and (IV) can be synthesized to have a per-metal r₁ relaxivity of greater than 4 mM⁻¹s⁻¹, preferably greater than 5 mM⁻¹s⁻¹, preferably greater than 6 mM⁻¹s⁻¹, preferably greater than 7 mM⁻¹s⁻¹, preferably greater than 8 mM⁻¹s⁻¹, preferably greater than 9 mM⁻¹s⁻¹, preferably greater than 10 mM⁻¹s⁻¹, or preferably greater than 11 mM⁻¹s⁻¹. When the compounds of Formulas (I) and (II) and (III) are synthesized such that least one of R¹ and R² comprises a blood plasma binding moiety, such as an albumin binding moiety, or when the compounds of formula (IV) are synthesized such that least one of R¹¹ and R¹³ comprises a blood plasma binding moiety, such as an albumin binding moiety, the compounds can have a per-metal r₁ relaxivity of greater than 5 mM⁻¹s⁻¹, or preferably greater than 10 mM⁻¹s⁻¹, or preferably greater than 15 mM⁻¹s⁻¹, or preferably greater than 20 mM⁻¹s⁻¹, or preferably greater than 25 mM⁻¹s⁻¹, or preferably greater than 30 mM⁻¹s⁻¹. Also, the compounds of Formulas (I) and (II) and (III) and (IV) can be synthesized to have a mean water residency time (τ_{M}) at 37° C. of 1 to 50 nanoseconds, more preferably 1 to 30 nanoseconds, more preferably 10 to 25 nanoseconds, or most preferably 15 to 20 nanoseconds.

Certain compounds are useful for synthesizing the compounds of Formulas (I) and (II). One particularly useful compound for synthesizing the compounds of Formulas (I) and (II), has the formula (VII):

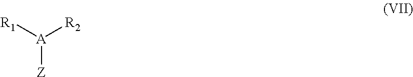

wherein A is a first amino acid residue, and Z is a chelator comprising a ring of atoms. The first amino acid residue is bonded to an atom of the ring of the chelator, and the first amino acid residue has a carbonyl group oxygen that can form a coordinate bond with a metal ion. R₁ is a moiety comprising hydrogen, or an amino acid residue, or combinations thereof, and R₂ is a moiety comprising hydrogen, or an amino acid residue, or combinations thereof. At least one of R₁ and R₂ comprises an amino acid residue, and at least one of R₁ and R₂ comprises a fluorenylmethyloxycarbonyl moiety.

The invention provides a method of increasing the r₁ relaxivity of a contrast agent. The contrast agent includes metal ion complexed to a chelator comprising a ring of atoms wherein the chelator forms at least one coordinate bond with the metal ion. In the method, a peptide scaffold is attached to the chelator such that the peptide scaffold has limited rotational freedom with respect to the chelator. The peptide scaffold includes a first amino acid residue, and the first amino acid residue is bonded to an atom of the ring of the chelator. The first amino acid residue also has a carbonyl group oxygen that forms a coordinate bond with the metal ion. In one non-limiting version, the chelator is 1,4,7,10-tetraazacyclododecane-1,4,7-triacetic acid, the metal ion is Gd³⁺, and the first amino acid residue is alanine.

The invention provides a method for in vivo imaging of a subject. In the method, any of the compounds of Formulas (I) and (II) and (III) and (IV) are administered to the subject. One waits a time sufficient to allow the compound to accumulate at a tissue or cell site to be imaged, and the cells or tissues are imaged with a non-invasive imaging technique whose resolution is enhanced by the presence of the compound on or within the cells. The non-invasive imaging technique can be magnetic resonance imaging.

The invention provides a method of imaging a subject having been administered a dose of a contrast agent including any of the compounds of Formulas (I) and (II) and (III) and (IV). In the method, the subject is positioned in a magnet system configured to generate a polarizing magnetic field about at least a portion of a subject. A plurality of gradient coils configured to apply a gradient field to the polarizing magnetic field are energized. A radio frequency (RF) system configured to apply an excitation field to the subject and acquire magnetic resonance (MR) image data therefrom is controlled, and an image of the subject is reconstructed from the MR image data. Molecules in the subject that are subjected to the contrast agent have a modified one of a longitudinal relaxation period and a transverse relaxation period that is reflected in the image.

When any of the compounds of Formulas (I) and (II) and (III) and (IV) includes a molecular targeting group, other bioactive agents may be included in the compound. For example, a cytotoxic agent can be associated with any of the compounds of Formulas (I) and (II) and (III) and (IV). A cytotoxic agent is “associated” with a compound of the invention if the cytotoxic agent is directly or indirectly, physically or chemically bound to the compound. Non-limiting examples of chemical bonds include covalent bonds, ionic bonds, coordinate bonds, and hydrogen bonds. Indirect bonding can include the use of a group of atoms (i.e., a linker) that chemically links the cytotoxic agent and the compound. Non-limiting examples of physical bonding include physical adsorption and absorption. The cytotoxic agent can be a cytotoxin (e.g., ricin, pseudomonas exotoxin, diphtheria toxin). The cytotoxic agent can be a chemotherapeutic agent (e.g., alkylating agents, antagonists, plant alkaloids, intercalating antibiotics, enzyme inhibitors, antimetabolites, mitotic inhibitors, growth factor inhibitors, cell cycle inhibitors, enzymes, biological response modifiers). The cytotoxic agent can be a radiation-emitter (e.g., phosphorus-32, phosphorus-33, bromine-77, yttrium-88, yttrium-90, molybdenum-99m, technetium-99m, indium-111, indium-131, iodine-123, iodine-124, iodine-125, iodine-131, lutetium-177, rhenium-186, rhenium-188, bismuth-212, bismuth-213, astatine-211).

We have investigated other chelators and chelate complexes useful in the invention. These chelate complexes could be attached to amino acid residues using the methods of the invention.

In another non-limiting example embodiment of the invention, the compound has the formula (VIII):

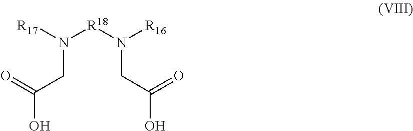

wherein R₁₆ is selected from substituted or unsubstituted alkyl carboxylates, substituted or unsubstituted cycloalkyl carboxylates, and substituted or unsubstituted heterocyclic carboxylates, wherein R₁₇ is selected from substituted or unsubstituted alkyl carboxylates, substituted or unsubstituted cycloalkyl carboxylates, and substituted or unsubstituted heterocyclic carboxylates, and wherein R₁₈ is selected from substituted or unsubstituted alkylenes and substituted or unsubstituted cycloalkylenes.

In one aspect, the compound of formula (VIII) is formulated such that R₁₈ is further selected from unsubstituted cycloalkylenes, such as cyclohexylene. In another aspect, R₁₆ is selected from unsubstituted alkyl carboxylates, such as C₁-C₂₀ alkyl carboxylate and in one case, carboxylate. In yet another aspect, R₁₇ is a carboxyalkylpyridine, such as carboxy-(C₁-C₂₀)alkyl-pyridine and in one case, carboxymethylpyridine. In another example, the compound of formula (VIII) is formulated such that R₁₆ is methyl carboxylate, R₁₇ is carboxymethylpyridine, and R₁₈ is cyclohexylene.

In another non-limiting example embodiment of the invention, the compound has the formula (IX):

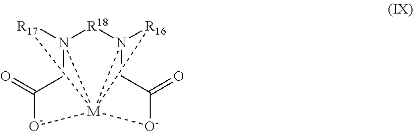

wherein R₁₆ is selected from substituted or unsubstituted alkyl carboxylates, substituted or unsubstituted cycloalkyl carboxylates, and substituted or unsubstituted heterocyclic carboxylates, wherein R₁₇ is selected from substituted or unsubstituted alkyl carboxylates, substituted or unsubstituted cycloalkyl carboxylates, and substituted or unsubstituted heterocyclic carboxylates, wherein R₁₈ is selected from substituted or unsubstituted alkylenes and substituted or unsubstituted cycloalkylenes, and wherein M is a metal ion.

In one aspect, the compound of formula (IX) is formulated such that R₁₈ is selected from unsubstituted cycloalkylenes, such as cyclohexylene. In another aspect, R₁₆ is selected from unsubstituted alkyl carboxylates, such as C₁-C₂₀ alkyl carboxylate, and in one case, methyl carboxylate. In yet another aspect, R₁₇ is a carboxyalkylpyridine, such as carboxy-(C₁-C₂₀)alkyl-pyridine, and in one case, carboxymethylpyridine. In another example, the compound of formula (IX) is formulated such that R₁₆ is methyl carboxylate, R₁₇ is carboxymethylpyridine, and R₁₈ is cyclohexylene.

The metal ion of formula (IX) may be selected based on a number of criteria. In one embodiment, the compound of formula (IX) is formulated such that the metal ion is selected from ions of gadolinium, europium, terbium, manganese, iron, ⁴⁵Ti, ⁵¹Mn, ⁵²Mn, ^{52m}Mn, ⁵²Fe, ⁶⁰Cu, ⁶¹Cu, ⁶²Cu, ⁶⁴Cu, ⁶⁷Cu, ⁶⁷Ga, ⁶⁸Ga, ⁷²As, ⁸⁶Y, ⁸⁹Zr, ⁹⁰Nb, ^{94m}Tc, ^{99m}Tc, ¹¹⁰In, ¹¹¹In, ¹¹³In, ¹⁷⁷Lu, ²⁰¹Tl, ²¹²Pb ²¹³Bi, or ²²⁵Ac. Alternatively, or in addition, the metal ion of formula (IX) is paramagnetic. In one aspect, the metal ion is selected from paramagnetic metal ions having atomic numbers 21-29, 43, 44, and 57-83.

In another aspect, the compound of formula (IX) is formulated to possess other desirable properties. For example, in one embodiment, the compound has a per-metal r₁ relaxivity of greater than 4 mM⁻¹s⁻¹, preferably greater than 5 mM⁻¹s⁻¹, preferably greater than 6 mM⁻¹s⁻¹, preferably greater than 7 mM⁻¹s⁻¹, preferably greater than 8 mM⁻¹s⁻¹, preferably greater than 9 mM⁻¹s⁻¹, preferably greater than 10 mM⁻¹s⁻¹, preferably greater than 11 mM⁻¹s⁻¹. In another aspect, the compound of formula (IX) has a mean water residency time of 5 to 30 nanoseconds, preferably 10 to 25 nanoseconds, preferably 15 to 20 nanoseconds.

In yet another embodiment, the compound of formula (IX) is employed as a contrast agent for magnetic resonance imaging. In one aspect, the metal ion of formula (IX) is Gd³⁺. In another aspect, the compound is heptadentate and/or the metal ion coordinates with two molecules of water.

The invention further encompasses a number of methods. One non-limiting method relates to increasing the r₁ relaxivity of a contrast agent having a metal ion complexed to a chelator including the step of attaching a peptide scaffold to the chelator such that the peptide scaffold has limited rotational freedom with respect to the chelator. In one aspect, the peptide scaffold includes a first amino acid residue that is bonded to an atom of the chelator. In another aspect, the first amino acid residue has a carbonyl group oxygen that forms a coordinate bond with the metal ion.

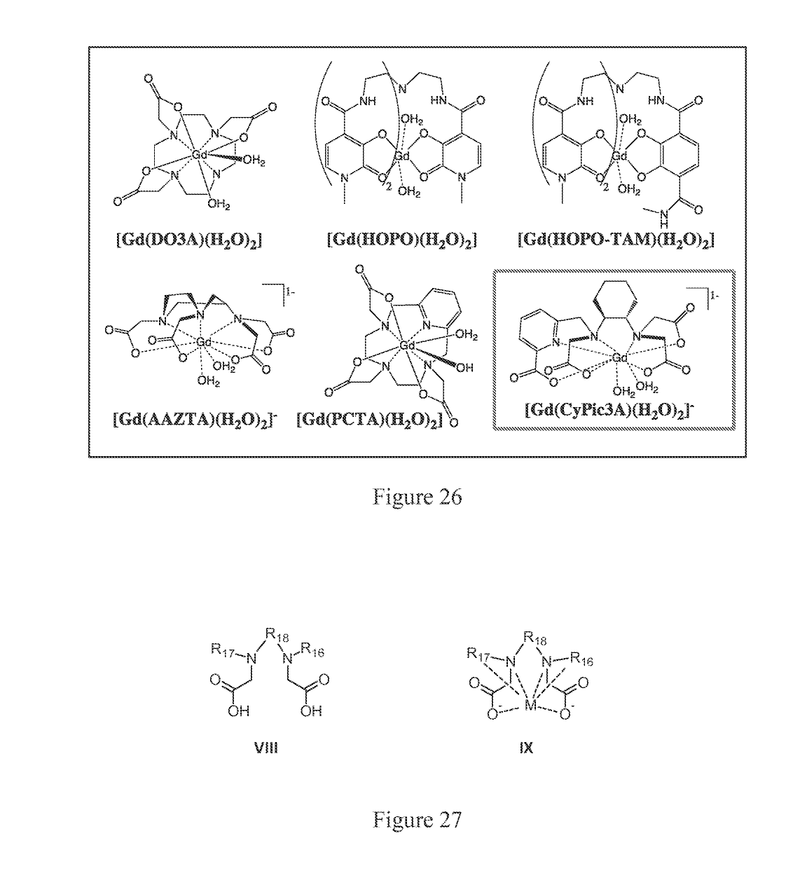

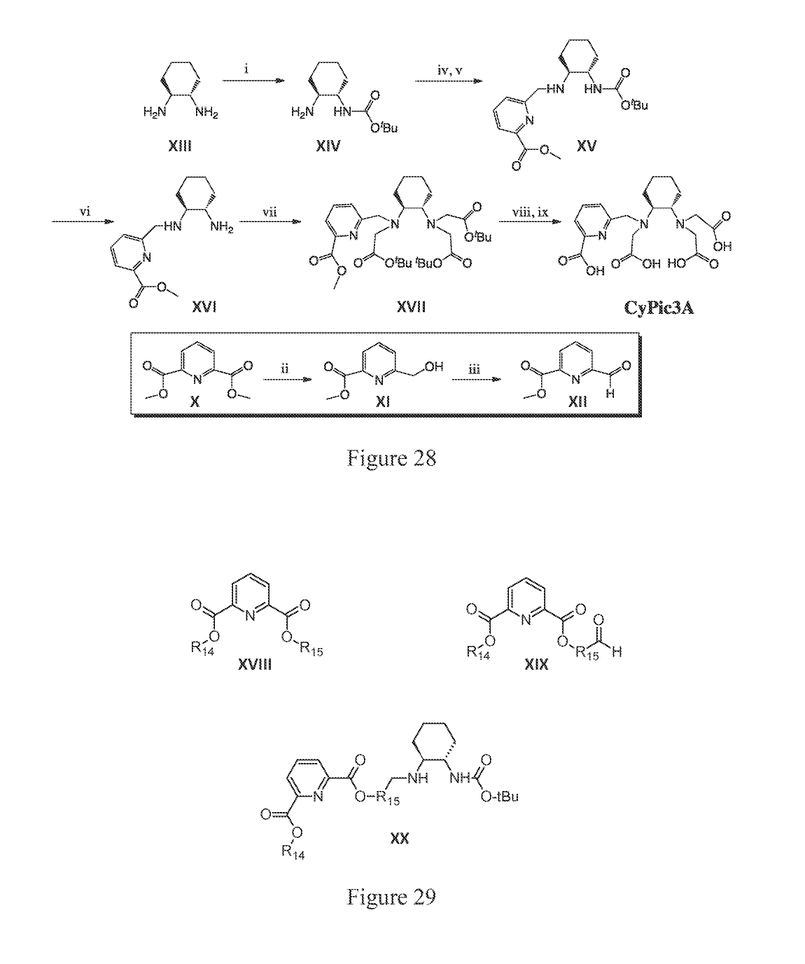

In one embodiment of the method, the chelator is CyPic3A as in FIG. 26. In another embodiment, the metal ion is Gd³⁺.

Yet another non-limiting method of the present invention for in vivo imaging of a subject includes the steps of (i) administering to the subject a compound such as the compound of formula (IX), (ii) waiting a time sufficient to allow the compound to accumulate at a tissue or cell site to be imaged; and (iii) imaging the cells or tissues with a non-invasive imaging technique whose resolution is enhanced by the presence of the compound on or within the cells. In one aspect, the non-invasive imaging technique is magnetic resonance imaging.

Still another non-limiting method relates to imaging a subject having been administered a dose of a contrast agent. The method includes the steps of (i) positioning the subject in a magnet system configured to generate a polarizing magnetic field about at least a portion of a subject; (ii) energizing a plurality of gradient coils configured to apply a gradient field to the polarizing magnetic field; (iii) controlling a radio frequency (RF) system configured to apply an excitation field to the subject and acquire magnetic resonance (MR) image data therefrom; and (iv) reconstructing an image of the subject from the MR image data. In one aspect, the contrast agent includes the compound of formula (IX). In another aspect, molecules in the subject that are subjected to the contrast agent have a modified one of a longitudinal relaxation period and a transverse relaxation period that is reflected in the image.

In addition to the compounds described above, another embodiment relates to compounds having the formula (VIII):

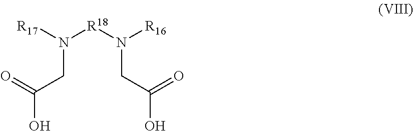

wherein R₁₆ is selected from substituted or unsubstituted alkyl carboxylates, substituted or unsubstituted cycloalkyl carboxylates, substituted or unsubstituted heterocyclic carboxylates, and amino acids, wherein R₁₇ is selected from substituted or unsubstituted alkyl carboxylates, substituted or unsubstituted cycloalkyl carboxylates, substituted or unsubstituted heterocyclic carboxylates, and amino acids and wherein R₁₈ is selected from substituted or unsubstituted alkylenes and substituted or unsubstituted cycloalkylenes.

Yet another embodiment relates to compounds having the formula (IX):

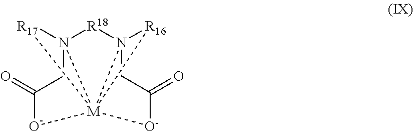

wherein R₁₆ is selected from substituted or unsubstituted alkyl carboxylates, substituted or unsubstituted cycloalkyl carboxylates, substituted or unsubstituted heterocyclic carboxylates, and amino acids, wherein R₁₇ is selected from substituted or unsubstituted alkyl carboxylates, substituted or unsubstituted cycloalkyl carboxylates, substituted or unsubstituted heterocyclic carboxylates, and amino acids, wherein R₁₈ is selected from substituted or unsubstituted alkylenes and substituted or unsubstituted cycloalkylenes, and wherein M is a metal ion.

In another non-limiting example embodiment of the invention, the compound has the formula (X):

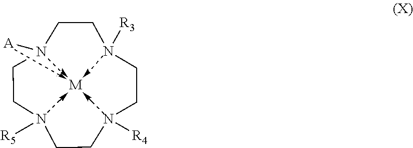

wherein A is selected from

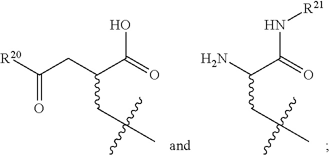

wherein R₃, R₄, and R₅ are independently selected from the group consisting of H, CH₂CO₂H, CH₂CH₂CO₂H, CH₂C(O)NR⁶R⁷, CH₂NHCOR⁶, CH₂C(O)N(OH)R⁶, CH₂C(O)NHSO₂R⁶, CH₂NHSO₂R⁶, CH₂N(OH)C(O)R⁶, CH₂P(R⁶)O₂R⁷, CH₂PO₃R⁶R⁷, wherein R⁶ and R⁷ are independently selected from the group consisting of H, CO₂H, C₁-C₆ alkyl, C₁₋₆CO₂H, CH(CO₂H)C₁₋₆CO₂H, C₁₋₆CF₃, C₁₋₆CCl₃, C₁₋₆CBr₃, C₁₋₆Cl₃, or C₁₋₆PO₃R⁹R¹⁰, wherein R⁹ and R¹⁰ are independently selected from the group consisting of H, CO₂H, C₁-C₆ alkyl, C₁₋₆CO₂H, CH(CO₂H)C₁₋₆CO₂H;

wherein R²⁰ and R²¹ are independently selected from

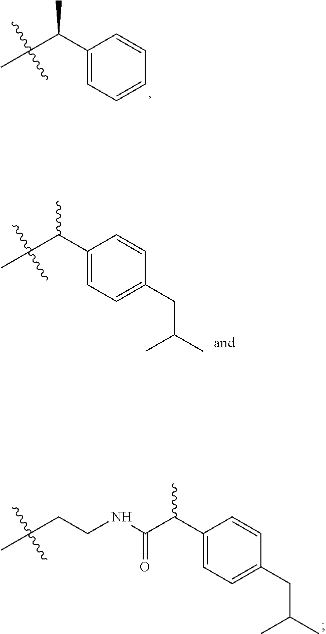

and

wherein M a metal ion.

The metal ion of formula (X) can be selected from ions of gadolinium, europium, terbium, manganese, iron, ⁴⁵Ti, ⁵¹Mn, ⁵²Mn, ^{52m}Mn, ⁵²Fe, ⁶⁰Cu, ⁶¹Cu, ⁶²Cu, ⁶⁴Cu, ⁶⁷Cu, ⁶⁷Ga, ⁶⁸Ga, ⁷²As, ⁸⁶Y, ⁸⁹Zr, ⁹⁰Nb, ^{94m}Tc, ^{99m}Tc, ¹¹⁰In, ¹¹¹In, ¹¹³In, ¹⁷⁷Lu, ²⁰¹Tl, ²¹²Pb ²¹³Bi, or ²²⁵Ac. The metal ion can be paramagnetic. The metal ion can be selected from paramagnetic metal ions having atomic numbers 21-29, 43, 44, and 57-83. The compound of formula (X) can have a per-metal r₁ relaxivity of greater than 3 mM⁻¹s⁻¹, preferably greater than 4 mM⁻¹s⁻¹, preferably greater than 5 mM⁻¹s⁻¹, preferably greater than 6 mM⁻¹s⁻¹. The compound of formula (X) can have a mean water residency time of 5 to 30 nanoseconds, preferably 5 to 20 nanoseconds, preferably 5 to 10 nanoseconds. The compound of formula (X) can be a contrast agent for magnetic resonance imaging. The metal ion of formula (X) can be Gd³⁺. In one structure of the compound of formula (X), R₃ is CH₂CO₂H, R₄ is CH₂PO₃R⁶R⁷, and R₅ is CH₂CO₂H wherein R⁶ and R⁷ are H.

Yet another non-limiting method of the present invention for in vivo imaging of a subject includes the steps of (i) administering to the subject a compound such as the compound of formula (X), (ii) waiting a time sufficient to allow the compound to accumulate at a tissue or cell site to be imaged; and (iii) imaging the cells or tissues with a non-invasive imaging technique whose resolution is enhanced by the presence of the compound on or within the cells. In one aspect, the non-invasive imaging technique is magnetic resonance imaging.

Still another non-limiting method relates to imaging a subject having been administered a dose of a contrast agent. The method includes the steps of (i) positioning the subject in a magnet system configured to generate a polarizing magnetic field about at least a portion of a subject; (ii) energizing a plurality of gradient coils configured to apply a gradient field to the polarizing magnetic field; (iii) controlling a radio frequency (RF) system configured to apply an excitation field to the subject and acquire magnetic resonance (MR) image data therefrom; and (iv) reconstructing an image of the subject from the MR image data. In one aspect, the contrast agent includes the compound of formula (X). In another aspect, molecules in the subject that are subjected to the contrast agent have a modified one of a longitudinal relaxation period and a transverse relaxation period that is reflected in the image.

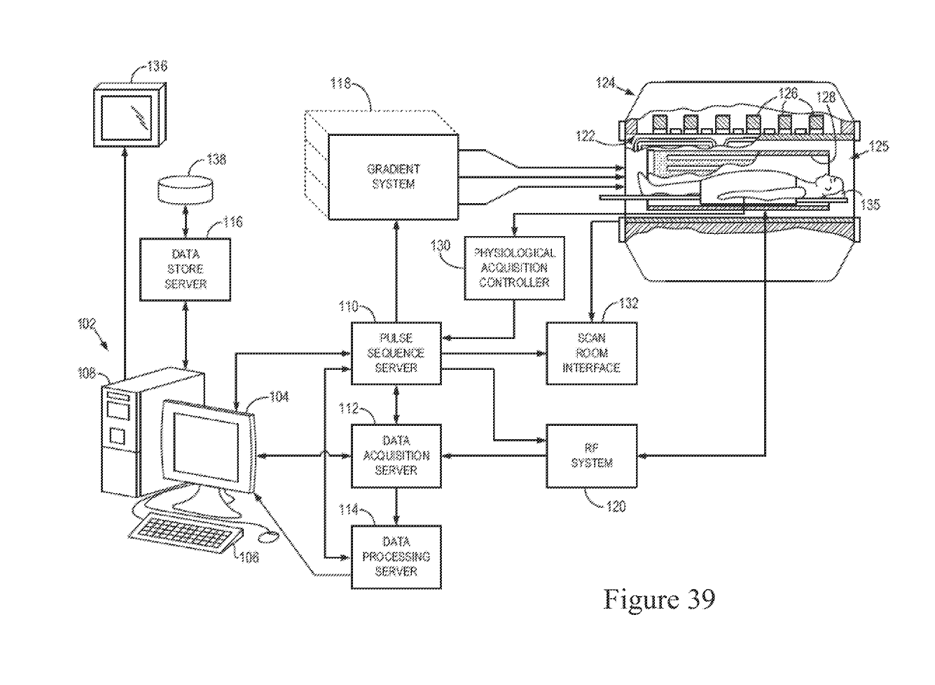

Referring to FIG. 39, any of the compounds of Formulas (I) and (II) and (III) and (IV) and (XIII) and (IX) and (X) can be used with a magnetic resonance imaging (“MRI”) system 100. The MRI system 100 includes a workstation 102 having a display 104 and a keyboard 106. The workstation 102 includes a processor 108, such as a commercially available programmable machine running a commercially available operating system. The workstation 102 provides the operator interface that enables scan prescriptions to be entered into the MRI system 100. The workstation 102 is coupled to four servers: a pulse sequence server 110; a data acquisition server 112; a data processing server 114, and a data store server 116. The workstation 102 and each server 110, 112, 114 and 116 are connected to communicate with each other.

The pulse sequence server 110 functions in response to instructions downloaded from the workstation 102 to operate a gradient system 118 and a radiofrequency (“RF”) system 120. Gradient waveforms necessary to perform the prescribed scan are produced and applied to the gradient system 118, which excites gradient coils in an assembly 122 to produce the magnetic field gradients Gx, Gy, and Gz used for position encoding MR signals. The gradient coil assembly 122 forms part of a magnet assembly 124 extending about a bore 125 formed there through and includes a polarizing magnet 126 and a whole-body RF coil 128.

RF excitation waveforms are applied to the RF coil 128, or a separate local coil (not shown), by the RF system 120 to perform the prescribed magnetic resonance pulse sequence. Responsive MR signals detected by the RF coil 128, or a separate local coil (not shown), are received by the RF system 120, amplified, demodulated, filtered, and digitized under direction of commands produced by the pulse sequence server 110. The RF system 120 includes an RF transmitter for producing a wide variety of RF pulses used in MR pulse sequences. The RF transmitter is responsive to the scan prescription and direction from the pulse sequence server 110 to produce RF pulses of the desired frequency, phase, and pulse amplitude waveform. The generated RF pulses may be applied to the whole body RF coil 128 or to one or more local coils or coil arrays.

The RF system 120 also includes one or more RF receiver channels. Each RF receiver channel includes an RF amplifier that amplifies the MR signal received by the coil 128 to which it is connected, and a detector that detects and digitizes the I and Q quadrature components of the received MR signal. The magnitude of the received MR signal may thus be determined at any sampled point by the square root of the sum of the squares of the I and Q components:

M=\sqrt{I²+Q²} Eqn. (1);

and the phase of the received MR signal may also be determined:

$$φ = {tan}^{- 1} ( \frac{Q}{I} ) . \qquad Eqn . ( 2 )$$

The pulse sequence server 110 also optionally receives patient data from a physiological acquisition controller 130. The controller 130 receives signals from a number of different sensors connected to the patient, such as electrocardiograph (“ECG”) signals from electrodes, or respiratory signals from a bellows or other respiratory monitoring device. Such signals are typically used by the pulse sequence server 110 to synchronize, or “gate,” the performance of the scan with the subject's heart beat or respiration.

The digitized MR signal samples produced by the RF system 120 are received by the data acquisition server 112. The data acquisition server 112 operates in response to instructions downloaded from the workstation 102 to receive the real-time MR data and provide buffer storage, such that no data is lost by data overrun. In some scans, the data acquisition server 112 does little more than pass the acquired MR data to the data processor server 114. However, in scans that require information derived from acquired MR data to control the further performance of the scan, the data acquisition server 112 is programmed to produce such information and convey it to the pulse sequence server 110. For example, during prescans, MR data is acquired and used to calibrate the pulse sequence performed by the pulse sequence server 110.

The data processing server 114 receives MR data from the data acquisition server 112 and processes it in accordance with instructions downloaded from the workstation 102. Images reconstructed by the data processing server 114 are conveyed back to the workstation 102 where they are stored. Real-time images are stored in a data base memory cache (not shown), from which they may be output to operator display 112 or a display 136 that is located near the magnet assembly 124 for use by attending physicians. Batch mode images or selected real time images are stored in a host database on disc storage 138. When such images have been reconstructed and transferred to storage, the data processing server 114 notifies the data store server 116 on the workstation 102. The workstation 102 may be used by an operator to archive the images, produce films, or send the images via a network to other facilities.

The invention is further illustrated in the following Examples which are presented for purposes of illustration and not of limitation.

### EXAMPLES

### Example 1

This example discloses the use of a single amino acid Gd-complex as a modular tool for high relaxivity magnetic resonance (MR) contrast agent development.

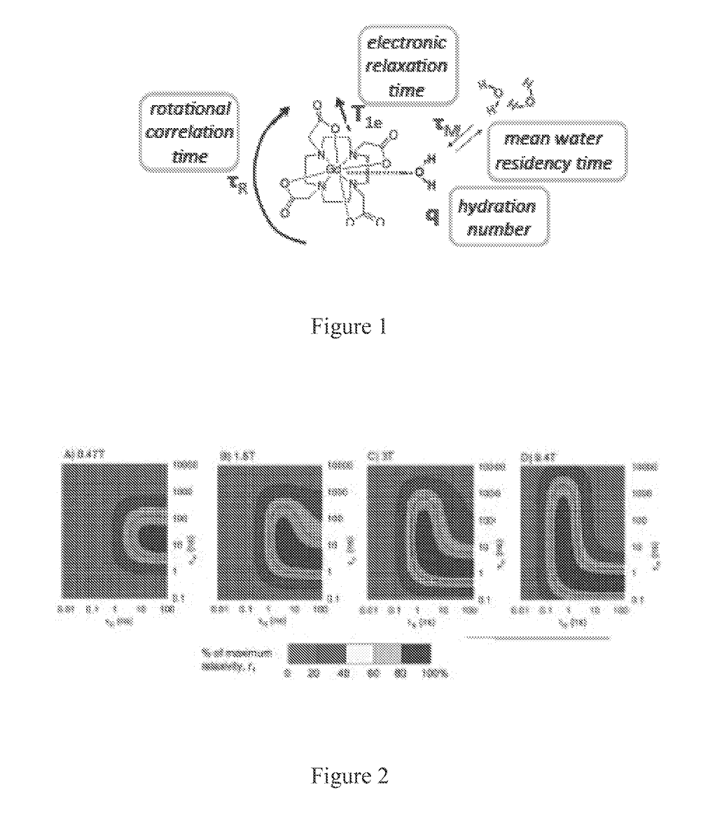

Introduction. MRI at high magnetic fields (B₀) benefits from an increased signal to noise ratio. For MR probes based on gadolinium (Gd, T₁ agents), the inherent relaxivity of tissue also increases with increasing B₀. Thus, if probe relaxivity (r₁) is field independent, the sensitivity of Gd-based probes should increase with B₀. Unfortunately, r₁ typically decreases with B₀ faster than the increase in baseline T₁. However, by controlling the rotational dynamics (τ_{R}) of the probe, it is possible to create high relaxivity probes with high r₁ at high fields (see FIGS. 1 and 2).

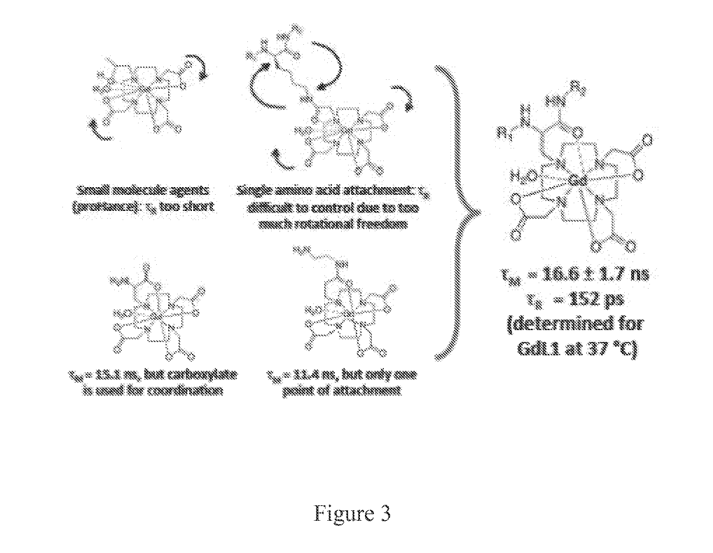

Concept. In order to optimize τ_{R} and τ_{M} for Gd-based high field imaging, we sought a system with an optimal τ_{M} and a tunable τ_{R}. Gd(DO3A-monopropionate) metal complexes have a near optimal τ_{M} in the range of 10-30 ns. Derivatization of the propionate arm with an alanine provides an easily derivatized, dually anchored metal complex with limited rotational freedom. Using standard Fmoc solid phase peptide synthesis, multimerization and control of τ_{R} is attainable (see FIG. 3).

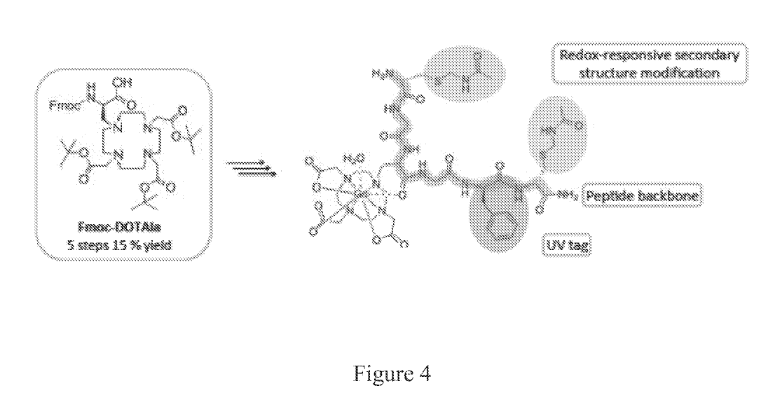

Synthesis and Relaxivity (1)—monomeric complex. Fmoc-DOTAla was synthesized in 5 steps and with 15% overall yield. Standard manual solid phase synthesis was employed to afford a monomeric prototype Gd-complex Gd(L1) (see FIG. 4). Gd(L1) was utilized to determine τ_{R} by temperature dependent measurement of transverse relaxation time T₂ using ¹⁷O-NMR (see FIG. 3).

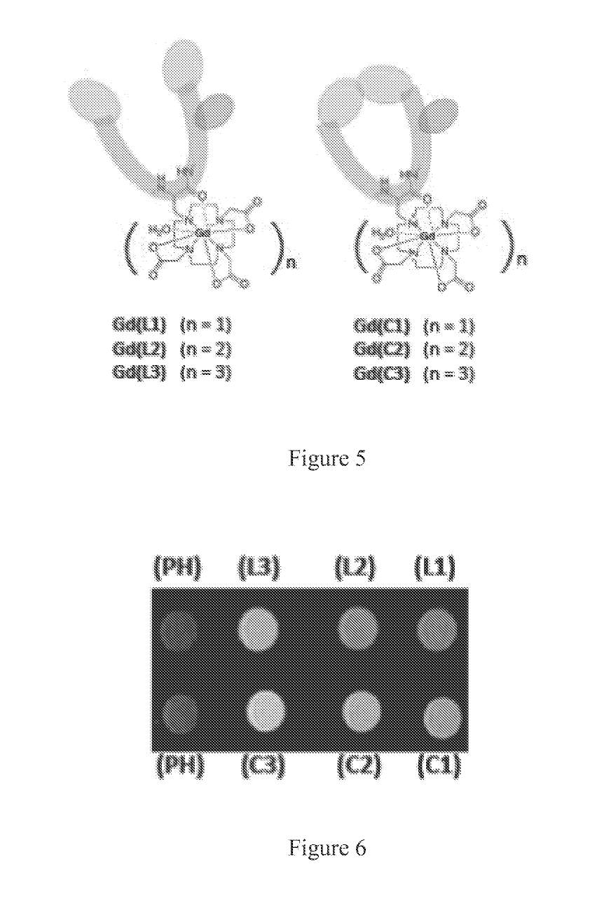

Synthesis and Relaxivity (2)—multimers. Standard manual solid phase synthesis was employed to afford linear, multimeric structures based on the DOTAla ligand system (Gd(L2), Gd(L3)). On-bead deprotection and cyclization of the cysteine residues provides cyclic systems (Gd(C1), Gd(C2), Gd(C3)) (see FIG. 5). Relaxivities were determined at 37° C.

Table 1 summarizes the relaxivity values measured in this example. Main numbers are given per Gd, numbers in parentheses are per molecule values. All numbers were determined at 37° C. using an inversion recovery sequence at concentrations ranging from 0.05 to 0.6 mM/Gd. Table 4 in Example 2 further expands the data of Table 1.

TABLE 1
Determined Relaxivities

| Complex | r1 [0.47 T] | r1 [1.4 T] | r1 [9.4 T] | r1 [11.7 T] |
|---|---|---|---|---|
| Gd (L1) | 8.12 | 7.47 | 5.76 | 4.89 |
| Gd (L2) | 10.7 (21.4) | 9.9 (19.8) | 6.14 (12.2) | 4.89 (9.78) |
| Gd (L3) | 13.17 (39.51) | 12.24 (36.72) | 6.13 (18.4) | 4.7 (14.1) |
| Gd (C1) | 8.3 | 6.7 | nd | nd |
| Gd (C2) | 13.1 (26.2) | 12.2 (24.4) | nd | nd |
| Gd (C3) | 13.38 (40.14) | 12.86 (38.58) | nd | nd |
| proHance | 4.33 | 3.21 | 3.03 | 2.94 |

Phantoms at 1.5 T. FIG. 6 shows T₁ weighted images acquired at 1.5 T, at 25° C. All complexes are at 0.1±0.014 mM/Gd concentration. ProHance (PH) is shown at equimolar concentration as a reference. TR: 5.2 ms, TE: 50 ms, flip angle: 60°.

Conclusions. We have successfully explored the synthesis and application of the novel Gd chelate DOTAla for mono- and multimeric structures (linear and cyclic). We confirmed the predicted mean water residency time of τ_{M}=16.6±1.7 ns, providing us with suitable water exchange kinetics for our purposes. Manual solid phase peptide synthesis allows us to vary size and therefore fine tune τ_{R}. The DOTAla system is a versatile single amino acid building block, ideal for the development of high relaxivity T₁ agents for high magnetic fields.

### Example 2

This example expands further on and provides additional detail regarding the work disclosed in Example 1.

### Summary

MR imaging at high magnetic fields benefits from an increased signal to noise ratio, however T₁ based MR contrast agents show decreasing relaxivity (r₁) at higher fields. High field, high relaxivity contrast agents can be designed by carefully controlling the rotational dynamics of the molecule. To this end, we investigated applications of the alanine analogue of Gd(DOTA), Gd(DOTAla). Fmoc protected DOTAla suitable for solid phase peptide synthesis was synthesized and integrated into polypeptide structures. Gd(III) coordination results in very rigid attachment of the metal chelate to the peptide backbone through both the amino acid sidechain and coordination of the amide carbonyl. Linear and cyclic monomers (GdL1, GdC1), dimers (Gd₂L2, Gd₂C2) and trimers (Gd₃L3, Gd₃C3) were prepared and relaxivities were determined at different field strengths ranging from 0.47 T to 11.7 T. Amide carbonyl coordination was indirectly confirmed by determination of the hydration number q for the EuL1 integrated into a peptide backbone, q=0.96±0.09. The water residency time of GdL1 at 37° C. was optimal for relaxivity, τ_{M}=17±2 ns. Increased molecular size leads to increased per Gd relaxivity (from r₁=7.5 for GdL1 to 12.9 mM⁻¹s⁻¹ for Gd₃L3 at 1.4 T, 37° C.). The cyclic, multimeric derivatives exhibited slightly higher relaxivities than the corresponding linearized multimers (Gd₂C2: r₁=10.5 mM⁻¹s⁻¹ versus Gd₂C2-red r₁=9 mM⁻¹s⁻¹ at 1.4 T, 37° C.). Overall, all six synthesized Gd complexes had higher relaxivities at low, intermediate and high fields than the clinically used small molecule contrast agent [Gd(HP-DO3A)(H₂O)].

### Introduction

Magnetic resonance imaging (MRI) is one of the most important modalities used for non-invasive investigation of disease in the clinic. MRI is the imaging technology of choice whenever high-resolution tissue contrast is required. Another advantage is the use of harmless magnetic fields for MRI as opposed to ionizing radiation in the case of CT.^{1,2} A large fraction of scans performed in the clinical setting are further enhanced by the use of contrast agents.³ Contrast agents shorten the relaxation times of water molecules in their proximity and increase tissue contrast on relaxation weighted imaging sequences.

Currently, most clinically employed contrast agents are non-target specific, small molecule gadolinium complexes which are able to increase the longitudinal relaxation rate 1/T₁ of water protons in the extracellular space.⁴ The extent to which a contrast agent can enhance relaxation depends on its concentration and its relaxivity (r₁), an inherent property of the molecule. Most approved contrast agents have low relaxivities (r₁) which makes them effective only at relatively high concentrations (≧0.1 mM).⁴

There has been a considerable research effort to increase the relaxivity of contrast agents.^{5, 6-9} Compounds with high relaxivity can be detected at lower doses¹⁰, or provide greater contrast at equivalent dose to compounds with lower relaxivity. Additionally, an attachment of a targeting moiety allows for target specific delivery of the contrast.¹⁰⁻¹² The clinically approved blood pool agent MS-325 (gadofosveset, Ablavar) is an example of a contrast agent with a high relaxivity¹³⁻¹⁴, this small molecular compound carries an albumin-targeting moiety and will display an over 8 fold increase in relaxivity at low fields once it is associated with human serum albumin (HSA)¹⁵, a blood plasma protein.

While 1.5 T remains the dominant field strength for clinical MRI, there is now a large installed base of 3 T scanners and the major equipment vendors also offer 7 T whole body human scanners. Small animal scanners operate almost exclusively at field strengths of 4.7 T and higher. The primary benefit of high field is the increased signal to noise ratio, which enables greater spatial resolution and reduced acquisition time. In addition, the inherent T₁ of tissue increases with increasing magnetic field.¹⁶ Thus, a contrast agent with equivalent relaxivity at a high and low field would provide much greater contrast at the high field. However, the relaxivity of many T₁-contrast agents decreases more rapidly with applied field than the inherent tissue T₁ increases.

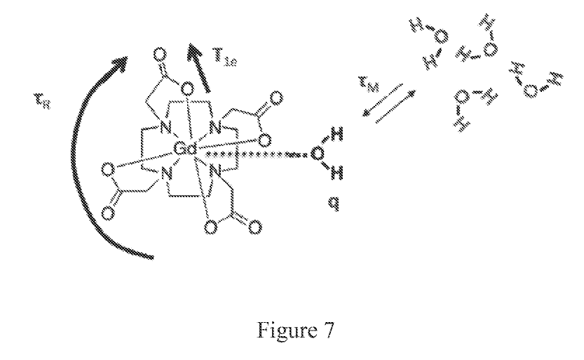

Relaxivity above 0.1 T depends on a variety of parameters, some of which are depicted in FIG. 7.¹⁹ As the magnetic field increases, the optimal correlation time, τ_{c}, for maximum possible relaxivity decreases, as it is inversely dependent on the proton Larmor frequency ω_{H}. While the contribution from the electronic relaxation time (T₁ₑ) is negligible at fields above 1.5 T, contributions from the mean water residency time (τ_{M}) and the rotational correlation time (τ_{R}) become the levers for generating high relaxivity Gd based agents.²⁰

For the design of high field, high relaxivity contrast agents, it is instructive to consider the equation for two site exchange written in terms of inner-sphere water relaxivity, Eq. (3) below, and the Solomon equation, Eq. (4) below, which describes the field dependence of T₁ relaxation of the coordinated inner-sphere water hydrogen atoms.³ Equation 3 teaches that the inner-sphere water relaxation time T_{1M} and the water residency time, τ_{M}, should be as short as possible. With regards to T_{1M}, equation 4 indicates that the correlation time should be as large as possible, but while still meeting the requirement of ω_{H} τ_{c}<1, where ω_{H} is the proton Larmor frequency and C is a constant. For a given Larmor frequency, there is an optimal correlation time. Unless water exchange is exceedingly fast (>10⁹ s⁻¹), the correlation time at 1.5 T and higher will essentially be the rotational correlation time, τ_{R}. If τ_{R} is very long (nanoseconds and longer), then relaxivity will be very high at low fields, but the condition ω_{H}τ_{c}>1 will also occur at lower fields and relaxivity will be low at high fields.

$$\begin{matrix}{r}_{1}^{IS} = \frac{q \text{/} [ {H}_{2} O ]}{{T}_{1 m} + {τ}_{M}} & Eq . ( 3 ) \\ 1 / {T}_{1 m} = C [ \frac{3 {τ}_{c}}{1 + {ω}_{H}^{2} {τ}_{c}^{2}} ] & Eq . ( 4 )\end{matrix}$$

For instance, MS-325 was designed for high relaxivity at low fields (≦1.5 T). Serum albumin binding of MS-325 results in a very long τ_{R} resulting in high relaxivity at 1.5 T, but a precipitous decline in relaxivity with increasing field strength.^{21,22} Small molecule agents with very short correlation times, such as [Gd(DTPA)(H₂O)]²⁻ (Magnevist), display a modest relaxivity decrease with increasing field strength but exhibit relatively low relaxivity due to their rapid tumbling.²³

We have previously investigated interplay of water exchange and rotational correlation time for Gd-based T₁ agents at fields ranging from 0.47 to 9.4 T,²¹ and showed that the optimal ranges are 5<τ_{M}<25 ns and 0.5<τ_{R}<2 ns to yield high relaxivity over a range of fields. A number of compounds with a corresponding, intermediate τ_{R} value between 0.35-1 ns has been reported;^{18, 24, 25} however, none of these structures allow for the simple adjustment of τ_{R} without sacrificing rigidity of the Gd-complex or complete redesign of the entire scaffold. The work in this example describes synthesis and investigation of a unique, modular system, capable of the construction of a new generation of high relaxivity T₁ contrast agents for high magnetic fields. Peptide structure and Gd complex incorporation can be modified using solid phase peptide synthesis, without change of the local complex environment.

### Structural Design

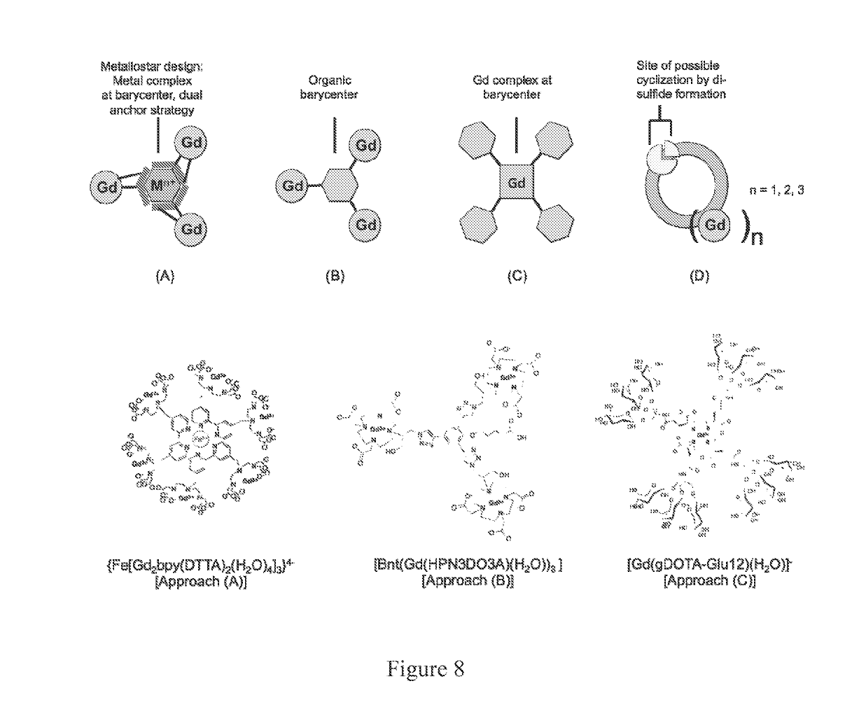

Control and optimization of τ_{R} requires rigid attachment of the corresponding Gd complex to a molecular construct of appropriate size. Conjugating the Gd complex to a targeting vector or molecular scaffold is typically done through a single linkage, and this results in fast internal motion about that linkage and concomitant lower relaxivity. Tweedle and coworkers introduced the dual anchor strategy,²⁶ which was also employed by Desreux and colleagues to rigidify attachment of the metal complex for the construction of fatty acid derivatized Gd(DOTA)²⁷. A similar, multi-site attachment strategy was employed for the design of metallostars, where a metallic barycenter is used as a point of attachment for multiple Gd(DTTA) type complexes (FIG. 8, approach A).²⁴ As attachment of multiple copies of the Gd complex increases size, the enhancement of τ_{R} combined with increase of the Gd-complex payload will further expedite molecular relaxivity.^{25, 28, 29} Meade and colleagues employed click chemistry to attach multiple Gd complexes to rigid, all-organic barycenters (FIG. 8, approach B).^{5, 25} Alternatively, Parker and coworkers showed that the rigid Gd complex can itself be placed at the barycenter of a molecule of variable size (FIG. 8, approach C).¹⁸ Most of these constructs display enhanced relaxivity at high fields compared to systems with either very short or very long τ_{R}. We discovered that a combination of 1) rigid attachment of the metal complex using the dual anchor strategy, 2) multimerization and 3) easy adjustment of molecular size would provide a construct highly suitable for high field applications.

The immediate coordination environment around the Gd complex influences important parameters such as kinetic inertness and water exchange kinetics. While q≧2 complexes can provide great relaxivity enhancement due to two or more possible sites of interaction for water molecules with the paramagnetic metal,³⁰⁻³⁴ only few have the kinetic inertness with respect to Gd dissociation/transchelation required for in vivo applications. For q=1, a myriad of kinetically inert Gd(DOTA) type complexes have been characterized.^{35, 36}

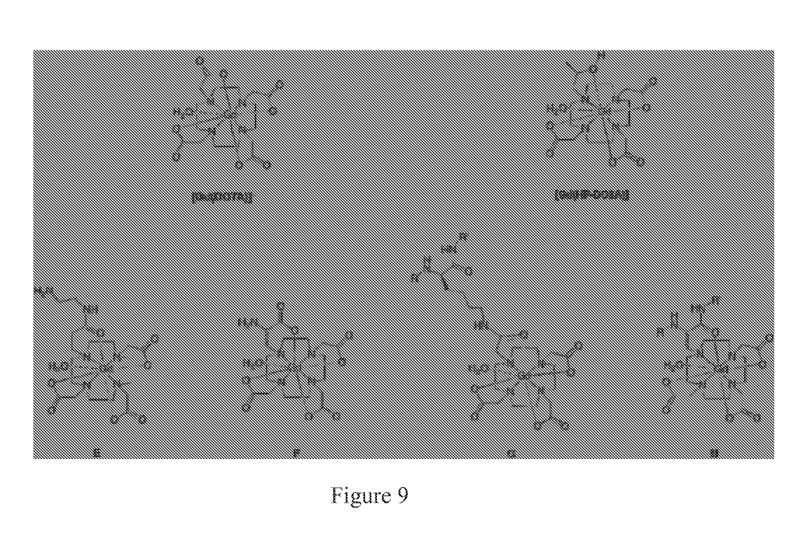

DOTA mono-proponiamide derivatives, where the amide forms a 6-membered chelate ring upon coordination of Gd(III), were found to have a mean water residency time of 10-20 ns (at 37° C.), which is within the ideal range required for our purposes (FIG. 9, compound E).³⁷ Geraldes and coworkers have reported the synthesis and investigation of Gd(DO3A-N-α-aminopropionate) (FIG. 9, Compound F).³⁸ We reasoned that derivatization and multimerization of DO3A-N-α-amino-propionate could be achieved by using an Fmoc-analogue of this system and standard peptide synthesis. See, for example, the work of Sherry et al. (FIG. 9, compound G),³⁹ as well as the work of Stephenson et al.⁴⁰ In our system, the complex is linked to the peptide backbone via the short methylene linkage of alanine sidechain. Gadolinium coordination of the amide carbonyl, which is also used for coupling to the polypeptide, provides the second point of attachment and results in a rigid incorporation of the complex into the polypeptide that should restrict internal motion and enable control over τ_{R}. This design would be capable of satisfying all our criteria: fast water exchange, tunable rotational dynamics; limited internal motion, and ease of derivatization using solid phase synthesis. Moreover, by using Gd(DOTAla) moiety itself to increase molecular size, the overall molecular relaxivity is increased via multimerization (FIG. 9, compound H).

### Results and Discussion

### Synthesis of Fmoc-DOTAla

For the use of DO3A-N-α-aminopropionate in solid phase peptide synthesis, construction of the corresponding Fmoc-derivative (“Fmoc-DOTAla-^{t}Bu₃”, compound 6) was required. Fmoc is easily deprotected under mildly basic conditions while the ligand-carboxylates remain protected⁴¹; hence, it is more suitable for our purposes than a potential Boc-derivative.⁵ As cyclen has high inherent basicity, the Fmoc protective group can only be introduced after alkylation of all secondary amines on cyclen.

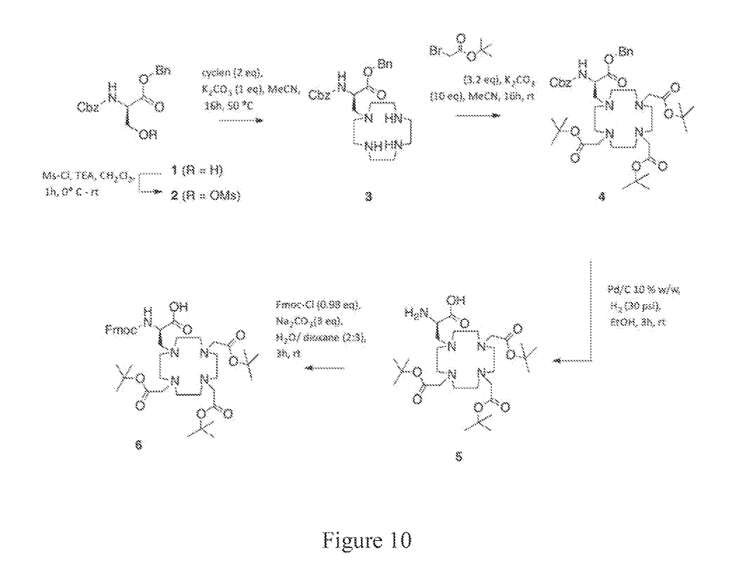

We employed a synthetic strategy in order to afford compound 6 in 15% over all yield after five synthetic steps (see FIG. 10). Commercially available serine derivative 1 was converted into the mesylate 2. Our initial attempted introduction of this sterically crowded synthon onto commercially available t-butyl protected DO3A failed. Instead, compound 2 was used for the mono-N-alkylation of cyclen. Compound 3 was further al kylated after removal of excess cyclen mesylate salt using tert-butyl bromoacetate in order to afford compound 4, which was isolated using preparative HPLC. Simultaneous removal of the benzyl and carboxybenzyl protective groups using H₂ and Pd/C yielded compound 5. Using Fmoc-Cl in a mixture of H₂O and dioxane under basic conditions for compound 3 results in the introduction of the desired Fmoc protective group. Compound 6 is purified using preparative HPLC. It was found that using extended reaction times over 12 hours leads to a considerable amount of side products of which some are more difficult to separate from the final product.

### Synthesis and Evaluation of Monomeric Metallopeptide

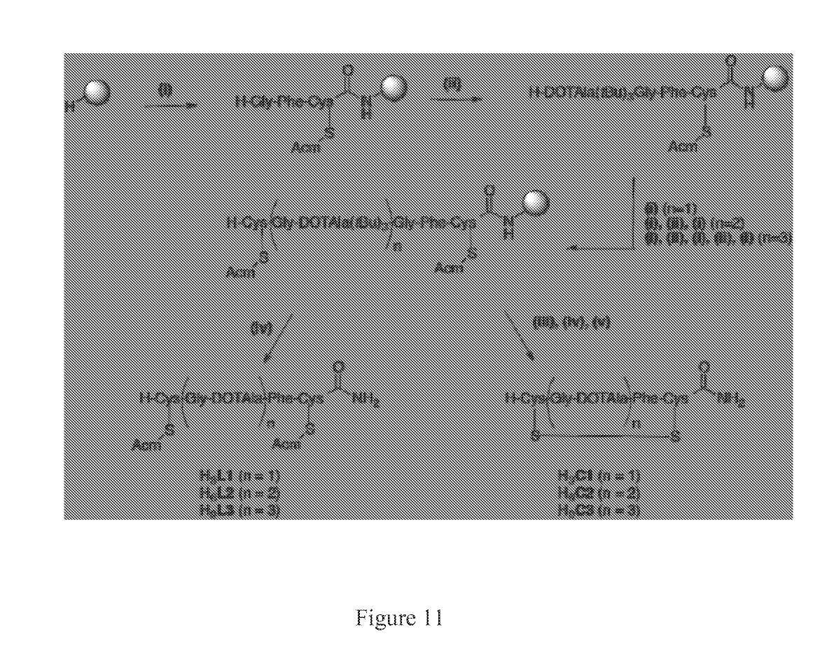

As a next step, we aimed to incorporate compound 6 into the linear model sequence H-Cys(Acm)-Gly-DOTAla-Gly-Phe-Cys(Acm)-CONH₂ (H₃L1, see FIG. 11). The corresponding Gd(III) complex would allow us to study water exchange kinetics, while the analogous Eu(III) complex provides information on the hydration number (q) at the metal center. Rather forcing conditions encompassing HATU in the presence of NMM in DMF were required for significant product formation. We synthesized H₃L1 using PEGA-Rink resin (grayish circle in FIG. 11) and standard manual solid phase synthesis (see FIG. 11).

Incorporation of a Phe residue was employed to provide a UV handle for detection and purification. Two Cys residues were used as the terminal amino acids, serving as potential sites of secondary structure modification through intramolecular disulfide bond formation. The tert-butyl esters were removed simultaneously with cleavage from the resin using a typical acidic cleavage cocktail (TFA:DDT:TIPS:H₂O (9.5:0.25:0.25:0.25)). The crude peptide H₃L1 was isolated using ether precipitation. Complexation with either GdCl₃.6H₂O or EuCl₃.6H₂O by mixing of aqueous solutions of the metal salt and the crude peptide at pH 3, followed by slow adjustment of the pH to 6.5 using an aqueous 0.1 M NaOH solution yielded the corresponding crude metal complex. The metallopeptide was purified using preparative HPLC.

This class of lanthanide-DOTA derivatives is typically 9-coordinate. If the amide carbonyl from the peptide backbone was coordinated to the lanthanide, then we would expect a single aqua co-ligand, i.e., q=1. The luminescence lifetime of the Eu(III) complex was measured in H₂O and D₂O. A custom-designed multimodal confocal imaging system built by Yaseen et al.⁴² was used to measure the luminescence lifetime of the Eu(III) excited state ⁵D₁ as previously reported.⁴³ Luminescence lifetimes were measured and averaged and used for the modified Horrocks equation⁴⁴ (equation 3), which accounts for the amide donor as one of the ligands.

q_{H2O}=1.2[(1/τ_{H2O}−1/τ_{D2O})−0.325] (3)

The value obtained for hydration number q was 0.96±0.09, which suggests that the carbonyl of the peptide backbone is indeed coordinated to the metal ion.

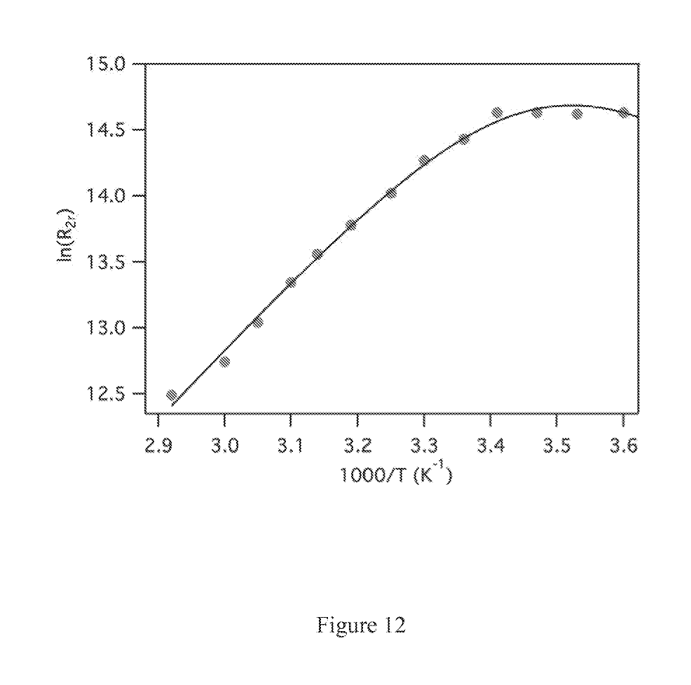

Water exchange kinetics for the inner-sphere water ligand were determined by measurement of the temperature dependence of the transverse relaxation time T₂ of H₂¹⁷O in the presence and absence of GdL1. The data in FIG. 12 were fit to a 4 parameter model.²² The water exchange rate at 310K, ³¹⁰kₑₓ, its activation enthalpy, ΔH^{‡}, the electronic relaxation time at 310K, ³¹⁰T₁ₑ, and its activation energy, ΔE^{‡} were iteratively varied to fit the observed reduced relaxation rate data R₂ᵣ. The hyperfine coupling constant was fixed at 3.8×10⁶ rad/s.⁴⁵

At the high field used, τ_{M} dominates the scalar correlation time and results in an accurate estimate of water exchange, while the relative contribution of T₁ₑ to ¹⁷O nuclear relaxation is much lower and this parameter is less well defined, see Table 2. The water residency time (τ_{M}=1/kₑₓ) was determined to be 17±2 ns at 310 K, which is similar to the Gd-DOTA-monopropionamide derivative reported by Geraldes and coworkers (Table 2, FIG. 12).³⁸ This similar water exchange rate is also consistent with the amide carbonyl as a donor. We further note that this water residency time is in the optimal range for high relaxivity at all field strengths.

TABLE 2
Water exchange kinetic parameters for GdL1 and
comparison with the Gd (III) complexes of
propionamide and propionate derivatives E and F (FIG. 9).
³¹⁰T₁ₑ = 160 ± 124 ns and AE^{‡} = 21 ± 18 kJ mol⁻¹ for GdL1.

| Gd | | | |
|---|---|---|---|
| complex | E³⁷ | F³⁸ | GdL1 |
| ³¹⁰kₑₓ × 10⁶ (s⁻¹) | 84 | 42 | 60 ± 6 |
| ΔH^{‡} (kJ mol⁻¹) | 34.0 | 19.1 | 41.7 ± 3.2 |
| ³¹⁰ T_{M} (ns) | 12 | 24 | 17 ± 2 |

This similar water exchange rate is also consistent with the amide carbonyl as a donor. We further note that this water residency time is in the optimal range for relaxivity at all field strengths.

### Multimeric Metallopeptides

GdL1 demonstrated that the GdDOTAla moiety could be incorporated into a peptide, and the resultant complex had the expected single inner-sphere water co-ligand with an optimal water exchange rate. However GdL1 is still a rather small molecule with a relatively short τ_{R}. In order to increase τ_{R} and enhance the molecular relaxivity, we also synthesized dimeric and trimeric structures. The cysteines were either left protected (‘linear’ structures) or were deprotected and used to induce intramolecular cyclization (‘cyclic’ structures) in order to highlight the possibility of secondary structure modification with our approach (see FIG. 11).

Multimers H₆L2 and H₉L3 were furnished using the same synthesis methodology as for the linear, model monomer peptide H₃L1. On-bead deprotection of the Acm (acetamidomethyl) protective group on the Cys amino acids using I₂ was done in order to afford the cyclicized analogues H₆C1, H₆C2 and H₉C3.⁴⁶ As cyclization was only 60% complete for compounds H₆C2 and H₉C3, cyclization was driven to completion using 2% DMSO in H₂O at pH 8 (see FIG. 11).⁴⁷ The Gd complexes GdL1, Gd₂L2 Gd₃L3, GdC1, Gd₂C2 and Gd₃C3 were formed and purified using the same methodology as described for the monomer. Isolated yields for the cyclic products were considerably lower due to intermolecular disulfide bond formation resulting in polymeric side products, which are separated by HPLC purification. All Gd complexes were characterized using LC ESI-MS.

### Kinetic Inertness

The development of new contrast agents requires compounds with high thermodynamic stability and kinetic inertness with respect to Gd dechelation. Tei et al. showed that a GdDOTA-monoproponiamide derivative had a very high stability constant, log K_{ML}=20.2,³⁷ and we expected that our system with the same donor set would exhibit similar thermodynamic stability. To address kinetic inertness, we measured the full transchelation of Gd(III) from the complexes GdL1, Gd₂L2 Gd₃L3 to a DTPA derivative with higher thermodynamic stability. Each of these complexes was challenged with one equivalent of the ligand of MS-325 (MS-325-L) on a per gadolinium basis (e.g. Gd₃L3 was challenged with 3 equivalents of MS-325-L).

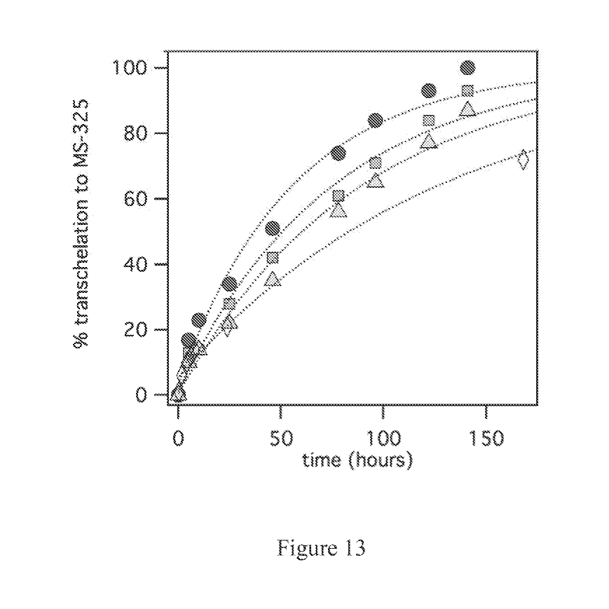

MS-325-L is a DTPA derivative with a biphenyl moiety that enables easy separation and monitoring of the free ligand from the MS-325 gadolinium complex by HPLC. FIG. 13 shows the conversion of MS-325-L to MS-325 as a function of time for the three metallopeptides at pH 3 (10 mM citrate buffer) and 37° C.³⁴

Transchelation was monitored using LC-MS, via formation of the MS-325 complex. For comparison, we also measured transchelation from the approved contrast agents [Gd(HP-DO3A)(H₂O)] (ProHance, gadoteridol) and [Gd(DTPA)(H₂O)]²⁻ (Magnevist, gadopentetate). Although thermodynamically favored, it is apparent from FIG. 13 that transchelation takes place over days even at pH 3. We estimated half-times for these transchelations (time to 50% of the equilibrium value). For the approved contrast agent [Gd(DTPA)(H₂O)]²⁻, the half-time was 25 minutes. On the other hand, the metallopeptides were much more inert with half-times in the 2-3 day range (Table 3). Transchelation was slowest for the trimer, followed by the dimer. The approved macrocyclic agent [Gd(HP-DO3A)(H₂O)] showed even slower transchelation kinetics. These results allowed us to conclude that multimers based on Gd(DOTAla) are also suitable for in vivo applications due to satisfactory kinetic inertness in comparison with clinically utilized Gd based agents. We were also able to confirm that multimerization has no detrimental effect on decomplexation of the metal complex, rather it appears to have a stabilizing effect.

TABLE 3
Half-times for Gd transchelation to MS-325 at pH 3, 37° C., with a
Gd:MS-325-L ratio of 1:1 at 0.1 mM Gd complex concentration.

| Gd complex | t_{1/2} [h] |
|---|---|
| GdL1 | 39 ± 3 |
| Gd₂L2 | 52 ± 3 |
| Gd₃L3 | 61 ± 4 |
| Gd (DO3A-HP) | 91 ± 6 |
| Gd (DTPA) | 0.42 ± 0.18 |

### Relaxivity

Per Gd relaxivities were determined by measuring T₁ at 37° C. using 20, 60, 200, 400 and 500 MHz spectrometers. Relaxivities for MS-325 (with and without the presence of HSA) as a reference compound with a long τ_{R} and [Gd(HP-DO3A)(H₂O)] as a reference for very short τ_{R} were also measured, and all the relaxivity data is tabulated in Table 4, together with results obtained from literature for the compounds with similar estimated τ_{R}. At low fields such as 0.47 and 1.4 T, the compounds with the highest rotational correlation times (MS-325/HSA and the trimers) exhibit the highest relaxivity. Additional rigidity through cyclization seems to provide only minor relaxivity increase for the dimeric and trimeric systems (Gd₂C2 and Gd₃C3).

TABLE 4
Measured per Gd relaxivities as a function of proton Larmor
frequency at 37° C. for the linear and cyclic systems described
herein along with Gd(HP-DO3A)(H₂O)] and MS-325 measured
in the presence and absence of 4.5% human serum albumin (HSA).
For comparison, literature data with examples of compounds
A (q = 2),¹⁷ B,⁵ C (data obtained at 25° C.)¹⁸ are included.

| | Relaxivity [mM⁻¹ s⁻¹] per Gd | | | | |
|---|---|---|---|---|---|
| | 20 | 60 | 200 | 400 | 500 |
| | MHz | MHz | MHz | MHz | MHz |
| GdL1 | 8.1 | 7.4 | 7 | 5.8 | 4.9 |
| Gd₂L2 | 10.8 | 9.9 | 8.3 | 6.1 | 4.9 |
| Gd₃L3 | 13.2 | 12.2 | 9 | 6.1 | 4.7 |
| GdC1 | 8.3 | 7.1 | 7.3 | 5.1 | 4.5 |
| Gd₂C2 | 11.4 | 10.6 | 7.5 | 5.7 | 4.5 |
| Gd₃C3 | 12.7 | 12.3 | 9.2 | 6.6 | 5.5 |
| [Gd(HP-DO3A)] | 4.3 | 3.2 | 3.6 | 3.0 | 2.9 |
| MS-325 | 6.8^{d} | 5.4^{d} | 5.7 | 4.8 | 4.7 |
| MS-325 w HSA | 42^{d} | 23.8^{d} | 5.0 | 4.1 | 3.7 |
| {Fe[Gd₂bpy(DTTA)₂(H₂O)₄]₃}⁴⁻ | 20.1^{a} | 26.8^{a} | 15.9^{a} | 8.3^{a} | n.a. |
| [Bnt(Gd(HPN3DO3A)(H₂O))₃] | ~15^{b} | 15.4^{b} | n.a. | 4.8^{b} | n.a. |
| [Gd(gDOTA-Glu12)(H₂O)]⁻ | 23.5^{c} | ~25^{c} | n.a. | n.a. | n.a. |

For Table 4: All data this work except ^{a}Ref.¹⁷; ^{b}Ref.⁵; ^{c}Ref.¹⁸; ^{d} data at 20 and 60 MHz from ref²²; n.a.; data not available.

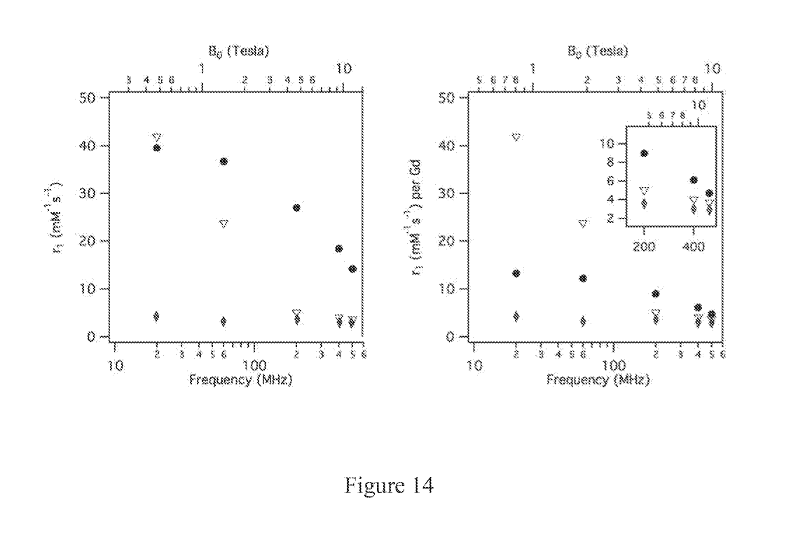

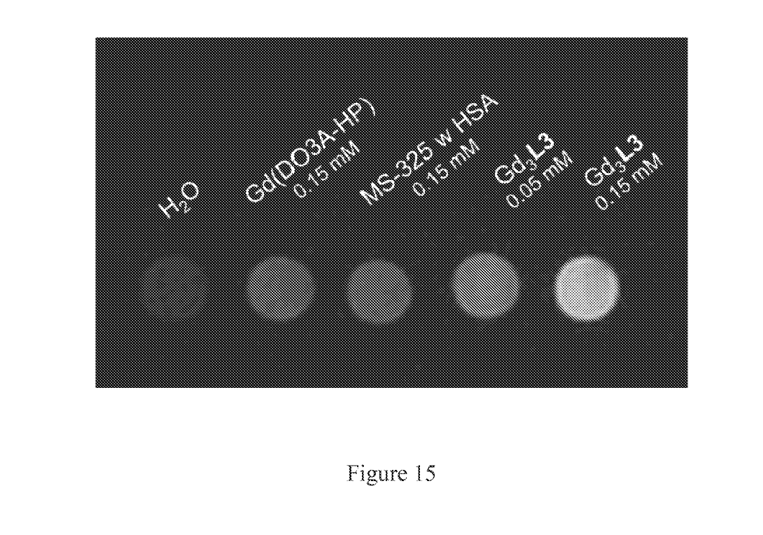

At intermediate field (4.7 T), only a moderate decrease in relaxivity is observed for the metallopeptides. In comparison, HSA-associated MS-325 exhibits a peak molecular relaxivity of above 40 mM⁻¹s⁻¹,⁴⁹ followed by rapid decrease in relaxivity upon increase of the magnetic field. Because we use the rigid GdDOTAla amino acid for multimerization, both the per Gd relaxivity and per molecule relaxivity increase with increased molecular size. FIG. 14A illustrates this effect where we plot the field dependent molecular relaxivity of GdL3 along with that of the approved contrast agents [Gd(HP-DO3A)(H₂O)] in PBS and MS-325 in the presence of excess HSA. At 0.47 T, the molecular relaxivities of GdL3 is similar to MS-325 in HSA solution. As the field is increased the molecular relaxivity of GdL3, with its intermediate rotational correlation time, becomes higher than that of MS-325/HSA: 50% higher at 1.4 T and 350-450% higher at fields from 4.7 to 11.7 T. The molecular relaxivity of GdL3 is 5-fold to 11-fold higher than that of [Gd(HP-DO3A)(H₂O)] at all fields measured. On a per Gd basis, the relaxivity of GdL3 is 50-220% higher than either HSA-bound MS-325 or [Gd(HP-DO3A)(H₂O)] at high fields (4.7-11.7 T), FIG. 14B.

In order to further illustrate this, we imaged a series of phantoms at 4.7 T (FIG. 15). Water is used as a reference for background, [Gd(HP-DO3A)(H₂O)] as an example of a compound with a short τ_{R}, and MS-325 bound to HSA as an example of a complex with a long τ_{R}. Gd₃L3 is shown at two different concentrations: either equimolar on a per Gd basis, or on a per molecule basis. It is evident, that Gd₃L3 provides better contrast at this field strength then either FDA approved compound, highlighting superiority in performance of our compound with intermediate τ_{R} at fields above 1.5 T. Under these conditions, the signal intensity of Gd₃L3 at equimolar Gd(III) ion concentrations was 65% greater than [Gd(HP-DO3A)(H₂O)] and 55% greater than MS-325/HSA. On a per molecule basis, the Gd₃L3 phantom was 190% and 170% brighter than Gd(HP-DO3A) and MS-325/HSA, respectively.

At 9.4 T, the per Gd relaxivities were 6.1 and 6.6 mM⁻¹s⁻¹ for the trimeric metallopeptides. These values are also found to be higher than the relaxivities measured at 9.4 T for previously reported trimeric compounds of similar composition and hydration number.^{5, 30} For compounds based on q=2 complexes, higher relaxivities can be obtained.¹⁷

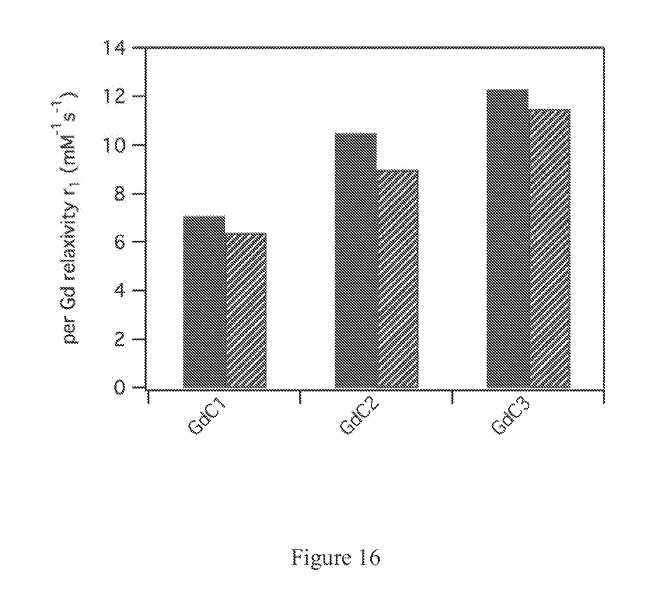

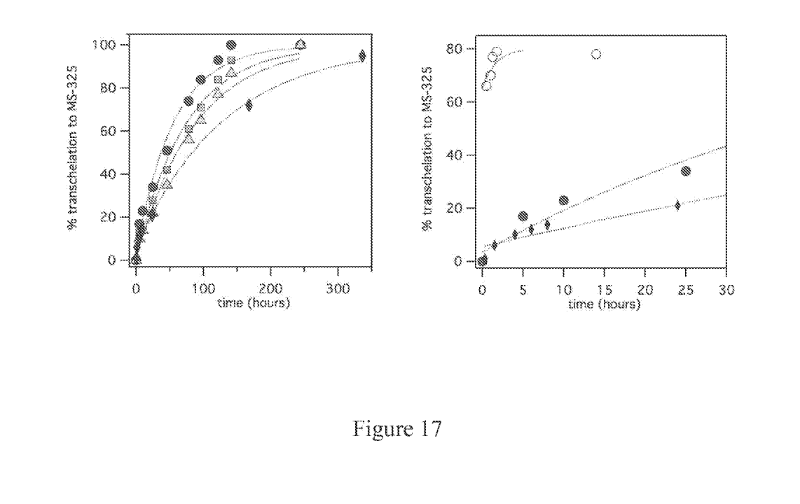

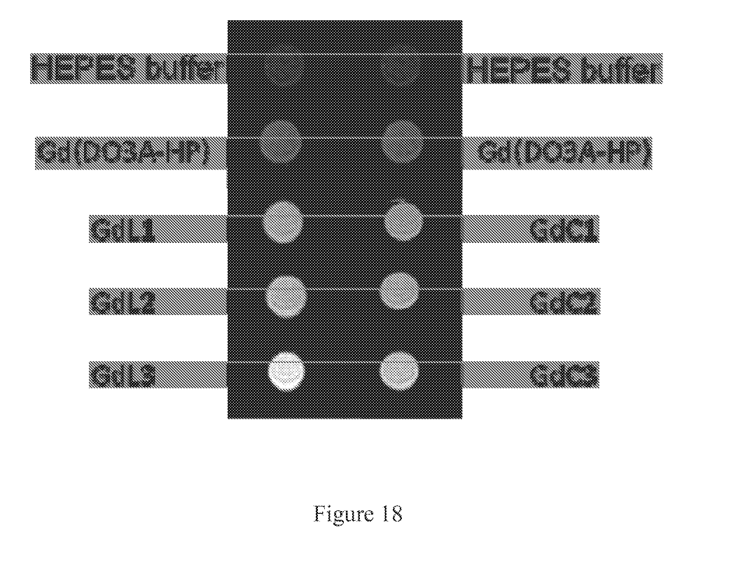

Investigation of the effect of tertiary structure on relaxivity, was done by examination of the effect of disulfide bond reduction on T₁ at 0.47 and 1.41 T. T₁ was measured for each sample at 37° C. and then the samples were incubated with 20 eq. TCEP for 30 minutes at room temperature to reduce the intramolecular disulfide bond and give the linear peptide. Subsequently, the T₁ values were remeasured and concentrations re-determined in order to calculate relaxivities. A slight decrease in relaxivity (7-14%) was observed for Gd₂C2-red (9 mM⁻¹s⁻¹) and Gd₃C3-red (11.5 mM⁻¹s⁻¹). Over all, reduction of the disulfide bond has only a slight effect on relaxivity (FIGS. 16-18 and Table 5).

TABLE 5
Measurement of relaxivity in dependence of
disulfide bond reduction (numerical values below).
Relaxivity per Gd [mM⁻¹ s⁻¹]

| | | |
|---|---|---|
| GdC1 | 7.1 | 6.4 |
| GdC2 | 10.5 | 9 |
| GdC3 | 12.3 | 11.5 |

We hypothesize that the large Gd-chelate side chain and the Gd(III) coordination by the amide carbonyl imposes defined structure to the peptide that dominates the over-all molecule structures for both the linear and the cyclic multimers. Introduction of a secondary structure modification such as the cyclization has only a marginal influence on the relaxivity. Nevertheless, facile introduction of the disulfide bridge by use of standard peptide synthesis methodology demonstrates the modularity of our system.

The high field relaxivities that we have obtained are consistent with an intermediate rotational correlation time. By assuming that the contributions of second-sphere and outer-sphere water can be estimated from a related q=0 complex,⁵⁰ we estimate τ_{R} of these metallopeptides to be in 150-600 ps range, based on the magnitude and field dependence of their relaxivities. More precise estimates of τ_{R} could be obtained by additional relaxation measurements using high resolution NMR with other Ln surrogates of Gd.⁵¹ Compared to the other multimers reported in Table 4, our relaxivities are similar. For a specific field strength, the rotational dynamics will dictate the optimal relaxivity. The modular amino acid approach presented here offers the possibility to tune such a high field relaxivity by systematically controlling the size and nuclearity of the complex.

### Conclusions

In conclusion, we were able to synthesize a single amino acid Gd chelate, Gd(DOTAla), suitable for solid phase peptide synthesis. The chelate is unique as it provides rigid and stable attachment of the metal complex to the rest of the molecule by using the amido-carbonyl of the corresponding peptide backbone as a point of attachment. Gd(DOTAla) when incorporated into a peptide exhibits one inner-sphere water ligand that has an optimal rate of water exchange for relaxometric purposes. The macrocyclic structure of the chelate provides high thermodynamic stability and kinetic inertness with respect to transchelation or Gd dissociation. The rigid incorporation of Gd(DOTAla) into a peptide scaffold allows design of contrast agents with defined rotational dynamics. Here, we described six new compounds containing 1-3 Gd(DOTAla) per peptide in a linear or cyclic peptide framework. By careful control of the rotational dynamics, it is possible to design contrast agents with high relaxivities at both low and high magnetic fields. These new contrast agents were superior to commercial contrast agents [Gd(HP-DO3A)(H₂O)] and MS-325/HSA at high fields. The modularity of design, the ease of solid phase synthesis, high kinetic inertness, and optimal water exchange rate renders the Gd(DOTAla) scaffold a suitable platform for the development of high field T₁ agents based on Gd.

### Experimental Section

### General Methods and Materials

¹H and ¹³C NMR spectra were recorded on a Varian 11.7 T NMR system equipped with a 5 mm broadband probe. Purification via HPLC of intermediates toward Fmoc-DOTAla was performed using method A: Injection of crude mixture onto preparative HPLC on a Rainin, Dynamax (column: 250 mm Polaris C18) by using A: 0.1% TFA in water, B: 0.1% TFA in MeCN, flow-rate 15 mL/min, from 5% B to 95% B over 20 minutes. Purification of Gd complexes was performed using method B: Injection of crude mixture onto analytical column (Phenomenex Luna, C18(2) 100/2 mm) using A: water, B: MeCN, flow-rate 0.8 mL/min, 15 min gradient from 2% B to 60% B over 15 min. Monitoring of UV absorption was done at 220 nm. HPLC purity analysis (both UV and MS detection) was carried out on an Agilent 1100 system (column: Phenomenex Luna, C18(2) 100/2 mm) with UV detection at 220, 254 and 280 nm by using a method C: A gradient of 95% A (0.1% formic acid in water) to 95% B (0.1% formic acid in MeCN), flow-rate 0.8 mL/min, 1 over 15 minutes. Kinetic inertness measurements were also carried out using the LCMS agilent system, using method D: A gradient of 95% A (ammonium formate, 20 mM, pH 6.8) with 5% (9:1 MeCN/20 mm ammonium formate) to 95% B (9:1 MeCN/20 mM ammonium formate), flow-rate 0.8 mL/min, 1 over 15 minutes.

The synthesis of ligands was carried out as shown in Schemes 1 and 2. Chemicals were supplied by Aldrich Chemical Co., Inc., and were used without further purification. Solvents (HPLC grade) were purchased from various commercial suppliers and used as received.

### Luminescence

Measurements were collected by using the confocal portion of a custom-designed multimodal microscope.^{42,43} Briefly, a continuous-wave diode laser (l=532 nm, B&W Tek) provided excitation light that was temporally gated by an electro-optical modulator (ConOptics, Danbury, Conn.) with extinction ratio of approximately 200 at 532 nm. The excitation beam passed through several conditioning optics, including a beam expander with pinhole spatial filter, polarizer, shutter, dichroic mirror, scan lens, and tube lens and a 20× magnification objective lens (XLumPlan FL, Olympus, NA=0.95). With the use of a customized control software and galvanometric scanners (Cambridge Technology, Inc. Lexington, Mass., USA), the excitation beam was guided to selected locations in the approximately 600 μm field of view. The emitted luminescence was descanned and collected by using an avalanche photodiode photon counting module (APD, SPCM-AQRH-10, Perkin-Elmer, Waltham, Mass., USA) sampled at 50 MHz with a high-speed DIO card (National Instruments, Austin, Tex., USA). Data were processed by using custom-written software in C and MATLAB (Mathworks, Natick, Mass., USA). Detected luminescent photons were binned into 50 ms long bins, to yield time-dependent phosphorescence decay profiles. With the use of a nonlinear least squares fitting routine, the resulting time-courses were fit with a single-exponential function. A sample's luminescence lifetime is equal to its fitted profile's calculated time constant.

(R)-tri-tert-butyl 2,2′,2″-(10-(3-(benzyloxy)-2-(((benzyloxy)carbonyl)amino)-3-oxopropyl)-1,4,7,10tetraazacyclododecane-1,4,7-triyl)triacetate (4). Cyclen (1.52 g, 8.8 mmol) was dissolved in MeCN (50 mL). K₂CO₃ (1 eq., 0.61 g) was added and the reaction mixture was preheated to 50° C. (R)-benzyl 2-(((benzyloxy)carbonyl)amino)-3-((methylsulfonyl)oxy)propanoate (2, 1.8 g, 4.4 mmol) was dissolved in MeCN (20 mL) and added dropwise to the preheated solution. After 16 hours, the precipitate was removed by filtration and the solvent evaporated. The residue was taken up in EtOAc and extracted twice with H₂O (80 mL), and once with brine (80 mL). The organic fraction was dried with Na₂SO₄ and the solvent was evaporated in vacuo to afford the crude mono-cyclen derivative (1.48 g, 3 mmol), which was resuspended in dry MeCN (50 mL) together with K₂CO₃ (10 eq., 4.24 g). tert-butyl bromoacetate (3.2 eq., 1.45 mL, 1.91 g) was added dropwise and the mixture was stirred for 16 hours at room temperature. The solvent was then removed and the residue was resuspended in EtOAc and extracted with H₂O and brine. The organic fraction was collected, dried with Na₂SO₄ and the solvent was evaporated in vacuo to afford the crude product which was purified using preparative HPLC, method A. Yield: 1.03 g (1.24 mmol, 28%). ¹H NMR (CDCl₃, 500 MHz, 298 K): δ=7.31-7.30 (m, Bn-H, 10H), 5.14-5.04 (m, CH₂-Bn, 4H), 4.75 (brs, α-CH, 1H), 3.75-3.05 (m, cyclen-H/N—CH₂—COO^{t}Bu, 24H), 1.47-1.42 (m, CH₃, 27H); ¹³C NMR (CDCl₃, 125 MHz, 303 K): δ=167.8, 167.7, 160.9, 160.7, 136.2, 134.8, 128.6, 128.5, 128.2, 127.9, 119.5, 117.2, 114.9, 112.6, 83.3, 68.0, 67.2, 55.0, 54.7, 50.9, 50.15, 27.9; LC/MS (ESI⁺): C₄₄H₆₇N₅O₁₀ m/z: calcd. 826.5 [MH⁺]; found: 826.4 (MH⁺).

(R)-2-amino-3-(4,7,10-tris(2-(tert-butoxy)-2-oxoethyl)-1,4,7,10 tetraazacyclododecan-1-yl)propanoic acid (5). Compound 4 (5.5 g, 6.7 mmol) was dissolved in EtOH (600 mL). Pd/C (2.9 g, 10% w/w) was added to afford a slurry which was subjected to H₂ (35 psi) for 3 hours. The Pd/C was filtered off and the filtrate was reduced in vacuo to afford the product (3.85 g, 6.4 mmol) as a colorless oil which was used without further purification in the subsequent reaction step. ¹H NMR (CD₃OD, 500 MHz, 298 K): δ=4.18 (brs, α-CH, 1H), 4.85-3.15 (m, cyclen-H/N—CH₂—COO^{t}Bu, 24H), 1.53-1.50 (m, CH₃, 27H); ¹³C NMR (CD₃OD, 125 MHz, 303 K): δ=170.4, 161.4, 161.2, 83.4, 54.3, 50.1, 49.1, 26.9; LC/MS (ESI⁺): C₂₉H₅₅N₅O₈ m/z: calcd. 602.4 [MH⁺]; found: 602.5 (MH⁺).

(R)-2-((((9H-fluoren-9-yl)methoxy)carbonyl)amino)-3-(4,7,10-tris(2-(tert-butoxy)-2-oxoethyl)-1,4,7,10-tetraazacyclododecan-1-yl)propanoic acid (6). Compound 5 (2.395 g, 3.98 mmol) was dissolved in dioxane (60 mL). Na₂CO₃ (1.27 g, 11.9 mmol, 3 eq) was dissolved in H₂O. The two solutions were mixed and cooled to 0° C. Fmoc-Cl (1.125 g, 4.3 mmol) was dissolved in dioxane (5 mL) and added to the reaction mixture. The solution was allowed to warm to room temperature and stirred for 4 hours. The solvent was then removed and the solid residue was dissolved in MeCN. The residual solid was filtered off and the filtrate was purified using preparative HPLC, method A. The product fractions were pooled and the solvent was removed in vacuo to afford the clean product as a white solid (1.81 g, 2.2 mmol, 55%). ¹H NMR (CDCl₃, 500 MHz, 298 K): δ=7.30-7.26 (m, FmocAr—H, 10H), 4.76 (brs, α-CH, 1H), 4.30 (m, CH₂-Fmoc, 2H), 4.15 (q, CH-Fmoc, 1H), 3.75-3.05 (m, cyclen-H/N—CH₂—COO^{t}Bu, 24H), 1.45-1.36 (m, CH₃, 27H); ¹³C NMR (CDCl₃, 125 MHz, 303 K): δ=171.1, 169.7, 143.6, 141.2, 127.8, 127.2, 125.2, 119.9, 84.8, 83.3, 67.5, 54.0, 50.7, 48.3, 46.8, 27.8; LC/MS (ESI⁺): C₄₄H₆₅N₅O₁₀ m/z: calcd. 824.5 [MH⁺]; found: 824.4 (MH⁺).

### Solid-Phase Peptide Synthesis

Solid-phase peptide synthesis was carried out manually following standard Fmoc protocols using PEGA Rink amide resin. All peptide sequences were derived from one solid support 0.33 mmol scale using single step couplings of four equivalents of Fmoc-amino acids, two equivalents coupling agent (HATU) and 3 equivalents N-methylmorpholine (NMM) in DMF at room temperature (refer to scheme 2). Coupling with commercial amino acids was completed within 12 hours (Step i), while Fmoc-DOTAla was only used in a 1.5 equivalent excess and allowed to react with the free N terminus of the peptide for 48 hours (Step ii). The coupling step was followed by rinsing with DMF and deprotection with 20% piperidine in DMF for 2 hours. After subsequent thorough rinsing with DMF and dichloromethane, a small aliquot of solid support was removed from the batch and deprotected using cleavage cocktail (TFA:DDT:TIPS:Water (9.5:0.25:0.25:0.25)) room temperature for 2 hours. The resin was filtered off and the filtrate concentrated with a gentle nitrogen flow. The intermediate was precipitated with cold diethyl ether, collected and characterized by ESI-MS. If coupling was found to be complete, the next coupling step was initiated on the main peptide batch. Once a sequence was complete, the corresponding aliquot was removed from the main resin batch and completed by addition of the terminal Fmoc-Cysteine-S-Acm.

For cyclic sequences, treating the resin-bound peptide with 10 equivalents of I₂ in DMF for 6 hours completed side chain deprotection with simultaneous cyclicization of the Cys residues (Step iii). After thorough rinsing of the resin with DMF and dichloromethane following the final processing step on-bead (Fmoc deprotection for linear systems, I₂ cyclicization for cyclic sequences), the crude peptide was afforded by cleaving from the resin using the acidic cleavage cocktail (see above) and isolated by cold ether precipitation, redissolution in water and lyophilization (Step iv). Because the on-bead cyclization proceeds to only approximately 60% completion, the crude peptide is further cyclicized using 2% DMSO in basic H₂O (pH˜7.5). As epimerization occurs on the stereocenter of DOTAla, multiple peaks are detected for the corresponding diastereomers.

H₂N—C(Acm)PG-DOTAla-GC(Acm)CONH₂ (H₃L1), HPLC: R=2.4/3.1 min, MS-ESI: m/z: 1043.4 (calcd. 1043.3) [M+H]⁺.

H₂N—C(Acm)PG-DOTAla-G-DOTAla-GC(Acm)CONH₂ (H₆L2), HPLC: Rₜ=1.3/1.48 min, MS-ESI: m/z: 1514.6 (calcd. 1514.5) [M+H]⁺.

H₂N—C(Acm)PG-DOTAla-G-DOTAla-G-DOTAla-GC(Acm)CONH₂ (H₉L3), HPLC: Rₜ=1.21/1.35 min , MS-ESI: m/z: 994.0 (calcd. 994.2) [M+2H]²⁺.

H₂N—C(S^{cycl})PG-DOTAla-GC(S^{cycl})CONH₂ (H₃C1), HPLC: R=1.35/1.6 min, MS-ESI: m/z: 898.4 (calcd. 898.3) [M+H]⁺.

H₂N—C(S^{cycl})PG-DOTAla-G-DOTAla-GC(S^{cycl})CONH₂ (H₆C2), HPLC: R=1.1/1.2 min, MS-ESI: m/z: 1372.5 (calcd. 1372.3) [M+H]⁺.

H₂N—C(S^{cycl})PG-DOTAla-G-DOTAla-G-DOTAla-GC(S^{cycl})CONH₂ (H₉C3), HPLC: R=1.24 min, MS-ESI: m/z: 922.95 (calcd. 922.8) [M+2H]²⁺.

Gadolinium Complex Formation. Complexes were prepared by adding GdCl₃ 6H₂O stock solution to a solution of ligand at pH 3 while stirring. The pH was gradually adjusted to pH 6.5 using 0.1 M NaOH solution. Complete complex formation was checked by LCMS (no residual ligand detectable). The solution was filtered and purified using preparative HPLC, method B. The Eu(III) complex is formed in analogous fashion.

H₂N—C(Acm)PG-DOTAla(Gd)-GC(Acm)CONH₂ (GdL1), HPLC: Rₜ=2.9/3.3 min, MS-ESI: m/z: 1197.3 (calcd. 1197.2) [M+H]⁺.

H₂N—C(Acm)PG-DOTAla(Gd)-G-DOTAla(Gd)-GC(Acm)CONH₂ (Gd₂L2), HPLC: Rₜ=2.6/3.0 min, MS-ESI: m/z 912.5 (calcd. 912.5) [M+2H]²⁺.

H₂N—C(Acm)PG-DOTAla(Gd)-G-DOTAla(Gd)-G-DOTAla(Gd)-GC(Acm)CONH₂ (Gd₃L3), HPLC: Rₜ=3.2 min, MS-ESI: m/z: 1225.95 (calcd. 1225.8) [M+2H]²⁺.

H₂N—C(S^{cycl})PG-DOTAla(Gd)-GC(S^{cycl})CONH₂ (GdC1), HPLC: Rₜ=1.13 min, MS-ESI: m/z: 1053.2 (calcd. 1053.4) [M+H]⁺.

H₂N—C(S^{cycl})PG-DOTAla(Gd)-G-DOTAla(Gd)-GC(S^{cycl})CON H₂ (Gd₂C2), HPLC: R=1.25 min, MS-ESI: m/z 840.7 (calcd. 841.5) [M+2H]²⁺.

H₂N—C(S^{cycl})PG-DOTAla(Gd)-G-DOTAla(Gd)-G-DOTAla(Gd)-GC(S^{cycl})CONH₂ (Gd₃C3), HPLC: R=1.35/1.9 min, MS-ESI: m/z: 1153.8 (calcd. 1154.6) [M+2H]²⁺.

Reduction of disulfide bond for relaxivity measurements. Complex solutions of purified, cyclic Gd complexes (concentrations of 0.1-0.025 mM, 110 μL) in HEPES buffer (50 mM, pH 7.4) were mixed with TCEP solution (20 mM in HEPES, 10 μL) and incubated room temperature. Reduction was checked by LCMS analysis and found to be complete after 30 minutes.

H₂N—C(SH)PG-DOTAla(Gd)-GC(SH)CONH₂ (GdC1-red), HPLC: Rₜ=2.35 min, MS-ESI: m/z: 1055.2 (calcd. 1055.2) [M+H]⁺.

H₂N—C(SH)PG-DOTAla(Gd)-G-DOTAla(Gd)-GC(SH)CONH₂ (Gd₂C2-red), HPLC: R=2.8-3.1 min, MS-ESI: m/z 841.7 (calcd. 842.0) [M+2H]²⁺.

H₂N—C(SH)PG-DOTAla(Gd)-G-DOTAla(Gd)-G-DOTAla(Gd)-GC(SH)CONH₂ (Gd₃C3-red), HPLC: Rₜ=3.1-3.3 min, MS-ESI: m/z: 1155 (calcd. 1155) [M+2H]²⁺.

### Measurement of Kinetic Inertness

Measurement of kinetic inertness. Stock solutions of MS-325-L and GdL1, Gd₂L2 and Gd₃L3 were prepared in 50 mM citrate buffer at pH 3.0. MS-325-L was added to solutions of the Gd complexes and incubated at 37° C. The final concentrations of the metal complexes were 0.1 mM, while the concentration of MS-325-L was adjusted according to the amounts of Gd complexes per metallopeptide present. A 10 μL aliquot was removed for HPLC analysis and analyzed while the remainder of the solution was incubated at 37° C. A 10 μL aliquot was removed and analyzed at 5, 10, 25, 46, 78, 96, 122, 141 and 244 hours. As a reference, Gd(HP-DO3A) was subjected to MS-325-L under same conditions and measured at time points 0.3, 1.5, 4, 6, 8, 24, 168 and 336 hours.

Measurement of relaxivity. Longitudinal relaxation times T₁, were measured on Bruker Minispecs mq20 (0.47 T) and mq60 (1.41 T), a Bruker Bioscan horizontal bore 4.7 T, 9.4 T and 11.7 T Varian NMR spectrometers. T₁ was measured by using an inversion recovery method with 10 inversion time values ranging from 0.05×T₁ to 5×T₁. Relaxivity was calculated from a linear plot of 3 or 4 different concentrations (ranging from 0.01 to 0.5 mM, depending on amount of compound isolated) versus the corresponding inverse relaxation times. All samples were measured at 37° C. using either the internal temperature control of the instrument (0.47, 1.41, 9.4 and 11.7 T) or a warm air blower (4.7 T). MS-325/HSA was prepared in a 4.5% w/v solution of HSA (0.66 mM) in PBS. The MS-325 concentration (in presence of HSA) ranged from 0.05 to 0.15 mM.

¹⁷O NMR of GdL1 solution for determination of T_{M}. ¹⁷O NMR measurements of solutions were performed at 11.7 T on 150 μL samples contained in 2-mm-shigemi tubes inside a 5 mm standard NMR tube on a Varian spectrometer. Temperature was regulated by air flow controlled by a Varian VT unit. ¹⁷O transverse relaxation times of distilled water (pH 3) containing 5% enriched ¹⁷OH₂ or a 6.88 mM solution of GdLl (pH 7.4, 50 mM HEPES buffer) were measured using a CPMG sequence. The concentration of the sample was determined by ICP-MS. Reduced relaxation rates, 1/T₂ᵣ were calculated from the difference of 1/T₂ between the GdL1 sample and the water blank, and then divided by the mole fraction of coordinated water. The temperature dependence of 1/T₂ᵣ was fit to a 4-parameter model as previously described.²² The Gd—O hyperfine coupling constant, AM, was assumed to be 3.8×10⁶ rad/s,⁴⁵ the Gd—O distance was assumed to be 3.1 Å.⁵²

### References for Example 2

1. Young, I. R., Methods in Biomedical Magnetic Resonance Imaging and Spectroscopy. John Wiley & Sons Ltd.: Chichester, 2000.
2. Caravan, P., Acc. Chem. Res. 2009, 42, 851-862.
3. Caravan, P.; Ellison, J. J.; McMurry, T. J.; Lauffer, R. B., Chem. Rev. 1999, 99, 2293-2352.
4. Caravan, P., Chem. Soc. Rev. 2006, 35,512-523.
5. Mastarone, D. J.; Harrison, V. S. R.; Eckermann, A. L.; Parigi, G.; Meade, T. J., J. Am. Chem. Soc. 2011, 133, 5329-5337.
6. Garimella, P. D.; Datta, A.; Romanini, D. W.; Raymond, K. N.; Francis, M. B., J. Am. Chem. Soc. 2011, 133, 14704-14709.
7. Kielar, F.; Tei, L.; Terreno, E.; Botta, M., J. Am. Chem. Soc. 2010, 132, 7836-7837.
8. Costa, J.; TOth, E.; Helm, L.; Merbach, A. E., Inorg. Chem. 2005, 44, 4747-4755.
9. Aime, S.; Frullano, L.; Crich, S. G., Angew. Chem. Int. Ed. 2002, 41, 1017-1019.
10. Terreno, E.; Castelli, D. D.; Viale, A.; Aime, S., Chem. Rev. 2010, 110, 3019-2042.
11. Raymond, K. N.; Pierre, V. C., Bioconjugate Chem. 2005, 16, 3-8.
12. Major, J. L.; Meade, T. J., Acc. Chem. Res. 2009, 42, 893-903.
13. Lauffer, R. B.; Parmelee, D. J.; Dunham, S. U.; Ouellet, H. S.; Dolan, R. P.; Witte, S.; McMurry, T. J.; Walovitch, R. C., Radiology 1998, 207, 529-538.
14. McMurry, T. J.; Parmelee, D. J.; Sajiki, H. S., D. M.; Ouellet, H. S.; Walovitch, R. C.; Tyeklar, Z.; Dumas, S.; Bernard, P.; Nadler, S.; Midelfort, K.; Greenfield, M.; Troughton, J.; Lauffer, R. B., J. Med. Chem. 2002, 45, 3465-3474.
15. Caravan, P.; Cloutier, N. J.; Greenfield, M. T.; McDermid, S. A.; Dunham, S. U.; Bulte, J. W. M.; Amedio, J. C.; Looby, R. J.; Supkowski, R. M.; Horrocks, W. D.; McMurry, T. J.; Lauffer, R. B., J. Am. Chem. Soc. 2002, 124, 3152-3162.
16. Rooney, W. D.; Johnson, G.; Li, X.; Cohen, E. R.; Kim, S.-G.; Ugurbil, K.; Springer, C. S., MRM 2007, 57.
17. Livramento, J. B.; Sour, A.; Borel, A.; Merbach, A. E.; Tóth, E., Chem. Eur. J. 2006, 12, 989-1003.
18. Fulton, D. A.; Elemento, E. M.; Aime, S.; Chaabane, L.; Botta, M.; Parker, D., Chem. Commun. 2006, 1064-1066.
19. Caravan, P.; Zhang, Z., Eur. J. Inorg. Chem. 2012, 1916-1923.
20. Helm, L., Future Med. Chem. 2010, 2, 385-396.
21. Caravan, P.; Farrar, C. T.; Frullano, L.; Uppal, R., Contrast Media Mol. Imaging 2009, 4 89-100.
22. Caravan, P.; Parigi, G.; Chasse, J. M.; Cloutier, N. J.; Ellison, J. J.; Lauffer, R. B.; Luchinat, C.; McDermid, S. A.; Spiller, M.; McMurry, T. J., Inorg. Chem. 2007, 46, 6632-6639.
23. Rohrer, M.; Bauer, H.; Mintorovitch, J.; Requardt, M.; Weinmann, H., Invest. Radiol. 2005, 40, 715-724.
24. Paris, J.; Gameiro, C.; Humblet, V.; Mohapatra, P. K.; Jacques, V.; Desreux, J. F., Inorg. Chem. 2006, 45, 5092-5102.
25. Song, Y.; Kohlmeir, E. K.; Meade, T. J., J. Am. Chem. Soc. 2008, 130, 6662-6663.
26. Ranganathan, R. S.; Fernandez, M. E.; Kang, S. I.; Nunn, A. D.; Ratsep, P. C.; Pillai, K. M. R.; Zhang, X.; Tweedle, M. F., Inves. Radiol. 1998, 33, 779-797.
27. Vanasschen, C.; Bouslimani, N.; Thonon, D.; Desreux, J. F., Inorg. Chem. 2011, 50, 8946-8958.
28. Sieving, P. F.; Watson, A. D.; Rocklage, S. M., Bioconjugate Chem. 1990, 1, 65-71.
29. Aime, S.; Botta, M.; Crich, S. G.; Giovenzana, G.; Palmisano, G.; Sisti, M., Bioconjugate Chem. 1999, 10, 192-199.
30. Livramento, J. B.; Helm, L.; Sour, A.; O'Neil, C.; Merbach, A. E.; Tóth, É., Dalton Trans. 2008, 1195-1202.
31. Datta, A.; Raymond, K. N., Acc. Chem. Res. 2009, 42, 938-947.
32. Aime, S.; Calabi, L.; Cavallotti, C.; Gianolio, E.; Giovenzana, G. B.; Losi, P.; Maiocchi, A.; Palmisano, G.; Sisti, M., Inorg. Chem. 2004, 43, 7588-7590.
33. Mato-Iglesias, M.; Roca-Sabio, A.; Pálinkás, Z.; Esteban-Gómez, D.; Platas-Iglesias, C.; Tóth, É.; Blas, A. d.; Rodriguez-Blas, T., Inorg. Chem. 2008, 47, 7840-7851.
34. Tallec, G.; Imbert, D.; Fries, P. H.; Mazzanti, M., Dalton Trans. 2010, 39, 9490-9492.
35. Jaszberenyi, Z.; Toth, E.; Kalai, T.; Kiraly, R.; Burai, L.; Brucher, E.; Merbach, A. E.; Hideg, K., Dalton Trans. 2005, 694-701.
36. Villaraza, A. J. L.; Bumb, A.; Brechbiel, M. W., Chem. Rev. 2010, 110, 2921-2959.
37. Tei, L.; Gugliotta, G.; Baranyai, Z.; Botta, M., Dalton Trans. 2009, 9712-9714.
38. Ferreira, M. F.; Martins, A. F.; Martins, J. A.; Ferreira, P. M.; Toth, E.; Geraldes, C. F. G. C., Chem. Commun. 2009, 43, 6475-6477.
39. De Leon-Rodriguez, L. M.; Kovacs, Z.; Dieckmann, G. R.; Sherry, A. D., Chem. Eur. J. 2004, 10, 1149-1155.
40. Stephenson, K. A.; Zubieta, J.; Banerjee, S. R.; Levadala, M. K.; Taggart, L.; Ryan, L.; McFarlane, N.; Boreham, D. R.; Maresca, K. P.; Babich, J. W.; Valliant, J. F., Bioconjugate Chem. 2004, 15, 128-136.
41. Vuljanic, T.; Bergquist, K.-E.; Clausen, H.; Roy, S.; Kihlberg, J., Tetrahedron 1996, 52, 7983-8000.
42. Yaseen, M. A.; Srinivasan, V. J.; Sakadzic, S.; W. Wu, S. R.; Vinogradov, S. A.; Boas, D. A., Opt. Express. 2009, 17, 22341-22349.
43. Moriggi, L.; Yaseen, M. A.; Helm, L.; Caravan, P., Chem. Eur. J. 2012, 18, 3675-3686.
44. Beeby, A.; Clarkson, I. M.; Dickins, R. S.; Faulkner, S.; Parker, D.; Royle, L.; Sousa, A. S. d.; Williams, J. A. G.; Woods, M., J. Chem. Soc., Perkin Trans. 1999, 2, 493-503.
45. Powell, D. H.; Dhubhghaill, O. M. N.; Pubanz, D.; Helm, L.; Lebedev, Y. S.; Schlaepfer, W.; Merbach, A. E., J. Am. Chem. Soc. 1996, 118, 9333-9346.
46. Xiaobing, T.; Wickstrom, E., Org. Lett. 2002, 4, 4013-4016.
47. Annis, I.; Hargittai, B.; Barany, G., Meth. Enzymol. 1997, 289, 198-221.
48. Overoye-Chan, K.; Koerner, S.; Looby, R. J.; Kolodziej, A. F.; Zech, S. G.; Deng, Q.; Chasse, J. M.; McMurry, T. J.; Caravan, P., J. Am. Chem. Soc. 2008, 130, 6025-6039.
49. Eldredge, H. B.; Spiller, M.; Chasse, J. M.; Greenwood, M. T.; Caravan, P., Invest. Radiol. 2006, 41.
50. Dumas, S.; Jacques, V.; Sun, W.-C.; Troughton, J. S.; Welch, J. T.; Chasse, J. M.; Schmitt-Willich, H.; Caravan, P., Invest Radiol. 2010, 45, 600-612.
51. Zech, S. G.; Eldredge, H. B.; Lowe, M. P.; Caravan, P., Inorg. Chem. 2007, 46, 3576-3584.
52. Caravan, P.; Astashkin, A. V.; Raitsimring, A. M., Inorg. Chem. 2003, 42, 3972-3974.

### Example 3

This example discloses experimental data for DOTAla modular human serum albumin (HSA) binders.

### General Methods and Materials

¹H and ¹³C NMR spectra were recorded on a Varian 500 NMR system equipped with a 5 mm broadband probe. Longitudinal relaxation times, T₁, were measured by using the inversion recovery method on Bruker Minispecs. mq20 (20 MHz) and mq60 (60 MHz). Purification via HPLC of intermediates toward Fmoc-DOTAla was performed using method A: Injection of crude mixture onto preparative HPLC on a Rainin, Dynamax (column: 250 mm Kromasil C18) by using A: 0.1% TFA in water, B: 0.1% TFA in MeCN, flow-rate 15 mL/min, 1 over 23 minutes. HPLC purity analysis (both UV and MS detection) was carried out on an Agilent 1100 system (column: Phenomenex Luna, C18(2) 100/2 mm) with UV detection at 220, 254 and 280 nm by using a method B: A gradient of A(0.1% formic acid in water) to 95% B(0.1% formic acid in MeCN), flow-rate 0.8 mL/min, 1 over 15 minutes.

In order to measure HSA binding of the complexes, a 0.1 mM solution (determined by ICP-MS) of the corresponding Gd complex in 4.5% w/v HSA was prepared and pipetted into a Ultrafree-MC Microcentrifuge Filter (NMWL 5,000 Da, PLCC, Millipore). The mixture is incubated at 37° C. for 10 minutes and subsequently centrifuged at 12,000 rpm for 15 minutes. Binding is determined by measurement of Gd content in the filtrate by ICP-MS.

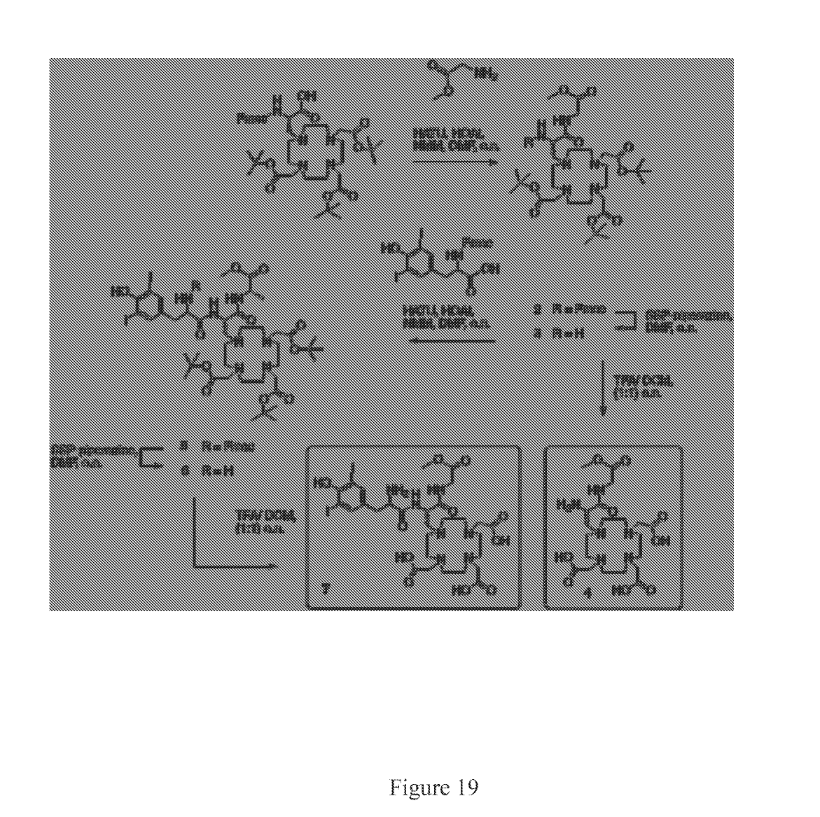

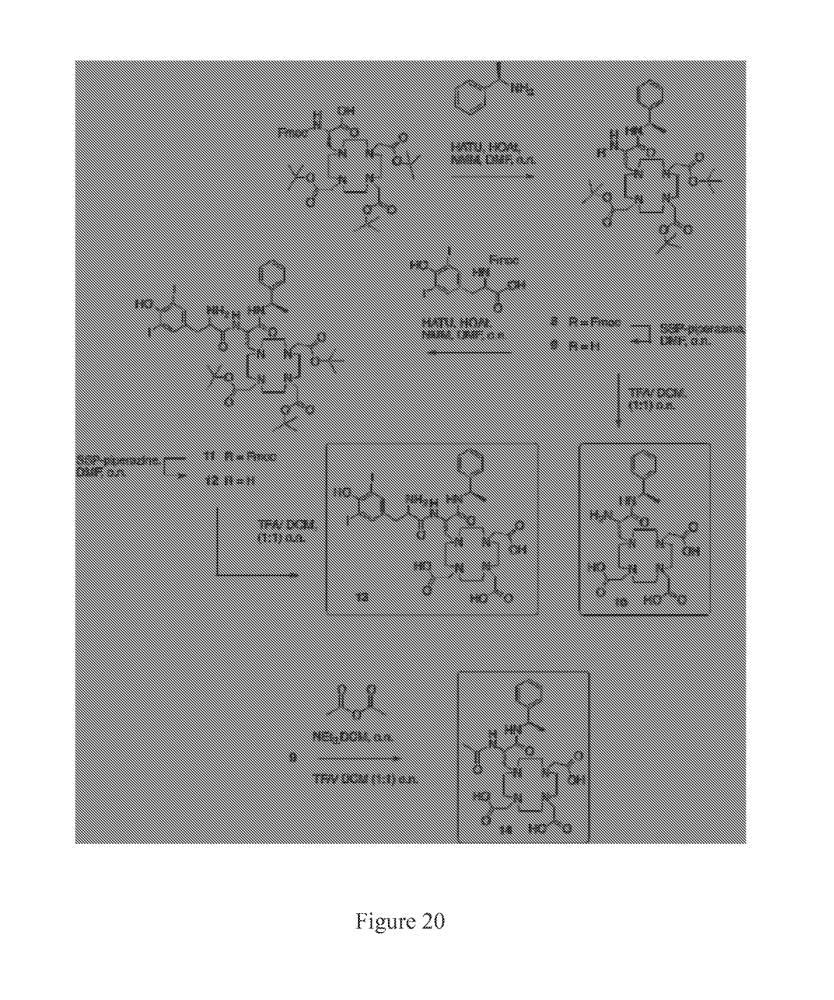

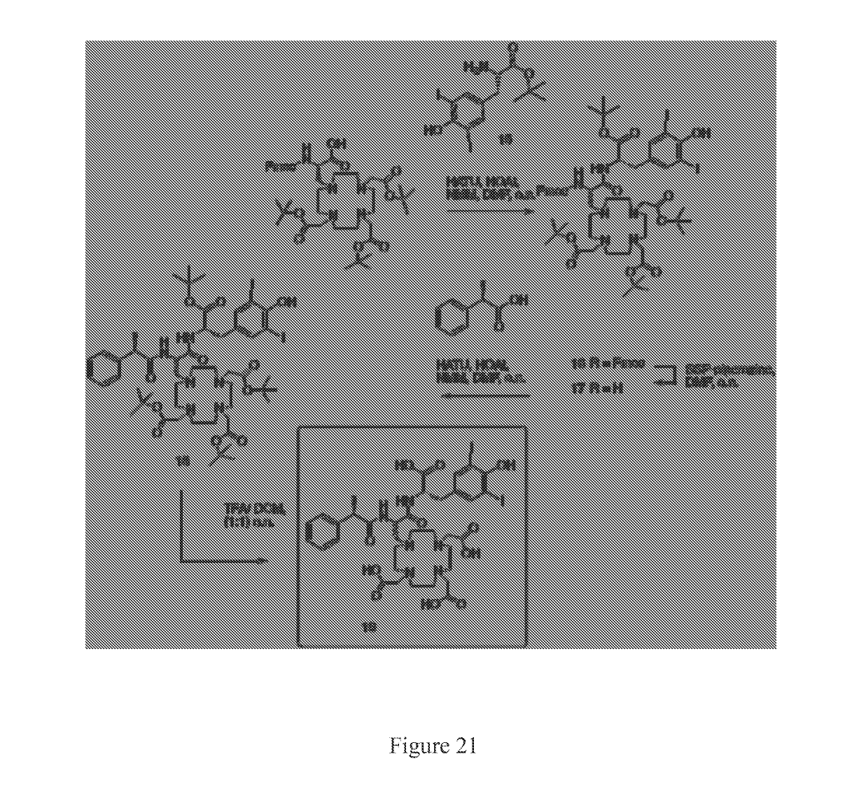

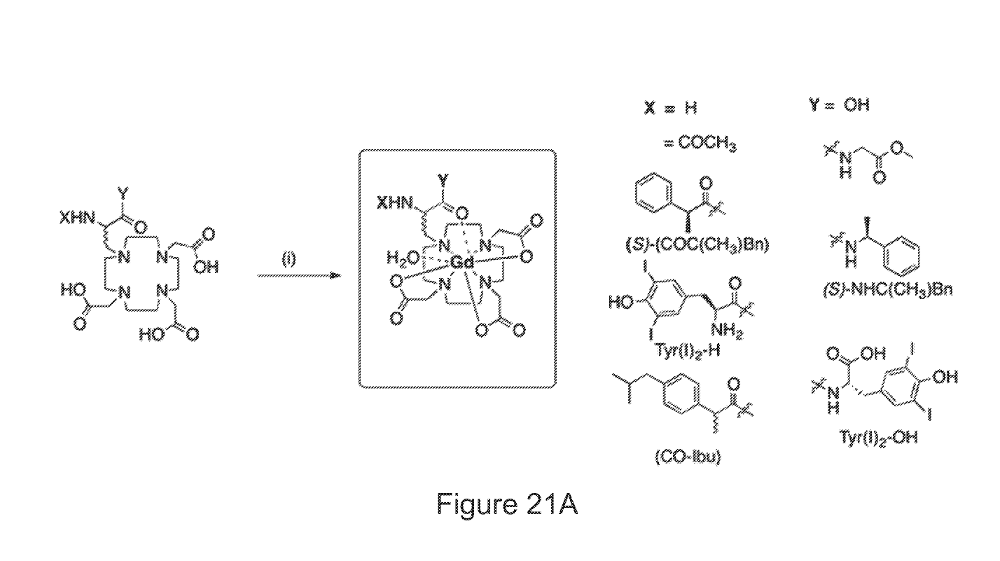

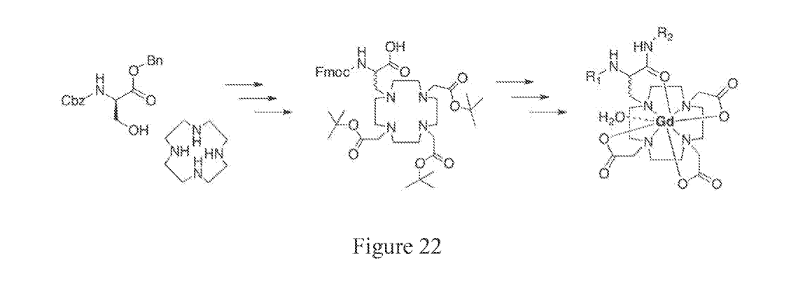

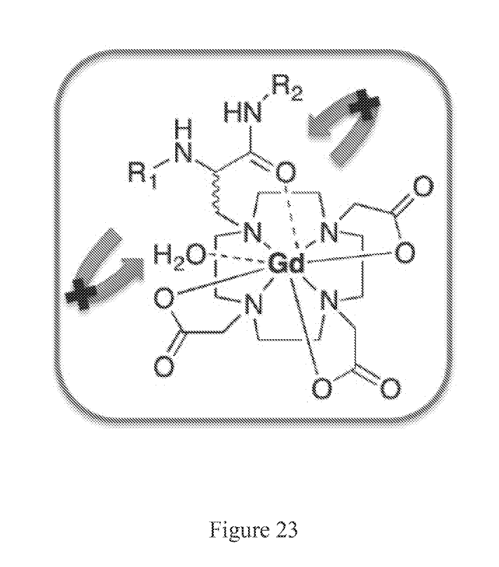

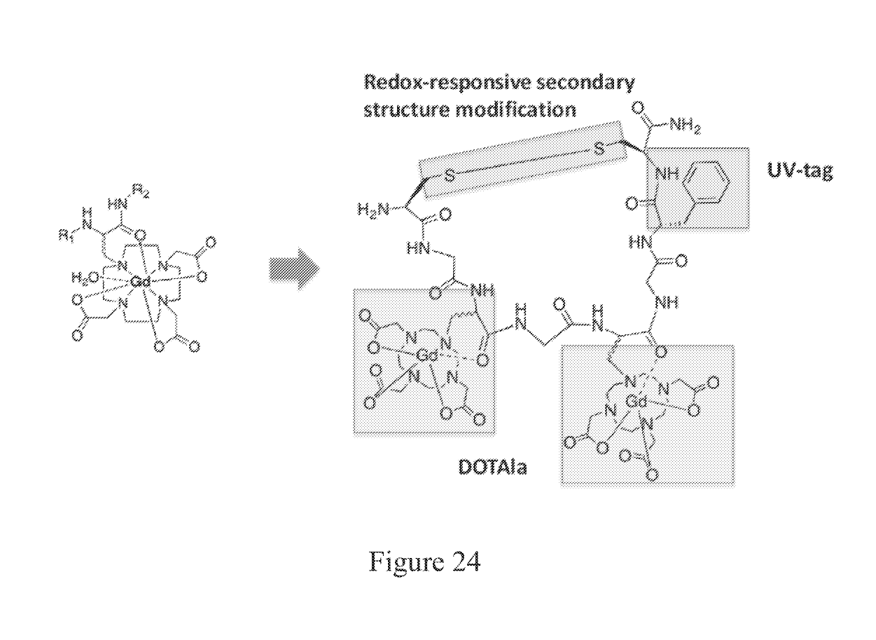

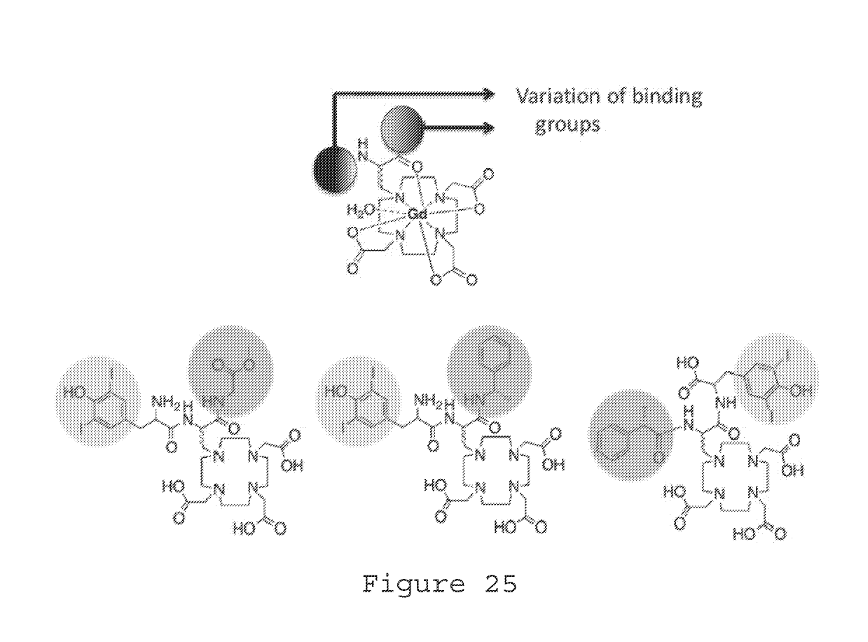

The synthesis of ligands was carried out as shown in Schemes 1, 2 and 3 (see FIGS. 19-21) and as shown in the scheme of FIG. 21A. Chemicals were supplied by Aldrich Chemical Co., Inc., and were used without further purification. Solvents (HPLC grade) were purchased from various commercial suppliers and used as received. Fmoc-DOTAla (1) was synthesized according to reference (TBS paper). Glycine methyl ester hydrochloride salt (Sigma), Fmoc-diiodo-tyrosine (Bachem), R-phenylethylamine (Sigma) and R-2-phenylpropionic acid (Sigma) were used as received.

### General Procedure 1

### Coupling of Fmoc-Protected Amino Acid with Free Amino Synthon

The free carboxylate (0.05 mmol) was dissolved in DMF (5 mL). HATU (1.5 eq.), HOAt (1.5 eq.) and NMM (2.5 eq) were added and the mixture was agitated for 5 minutes in order to activate the carboxylate. Subsequently, the corresponding free amine (1.5 eq.) was added to the mixture and the solution was stirred at room temperature over night. Reaction control via LCMS (method B, see above) was used in order to evaluate if product was present. The reaction mixture was purified using preparative HPLC (method A). Fractions containing the pure product were pooled, lyophilized and used for the subsequent reaction step after analysis.

### General Procedure 2

### Fmoc Deprotection of Intermediates

The Fmoc-protected intermediate synthon (0.01-0.05 mmol) was dissolved in DMF (1 mL) and added to a solution of solid-support-piperazine (SSP-pip, 10 eq.) suspended in DMF (5 mL). The mixture was stirred at room temperature over night. Reaction control via LCMS (method B, see above) was used in order to evaluate any residual starting material was present. Another batch of SSP-pip (10 eq.) was added if significant amounts of starting material were found to be present. Once the reaction was complete, the reaction mixture was filtered in order to remove the SSP-pip. The filtrate was used without further purification for the next reaction step if procedure 1 followed, or purified using preparative HPLC (method B), if no additional peptide couplings followed. For compounds 3, 6, 9, the solvent is removed and the intermediate used for the deprotection step without further purification. The only contaminant observed (1-((9H-fluoren-9-yl)methyl)piperazine) is removed via filtration of the subsequent reaction mixture.

### General Procedure 3 ((Bu Deprotection)

The starting material (0.01-0.02 mmol) was dissolved in a mixture of dichloromethane and trifluoroacetic acid (1:1, 2 mL) and stirred over night. LCMS provided reaction control. The solvent is removed in vacuo and the product is isolated as the trifluoroacetate salt.

### General Procedure 4 (Gd Complex Formation)

The ligand (0.01 mmol) was redissolved in H₂O. The pH was adjusted to 3 and GdCl₃.6H₂O (0.07 mmol) was added and the pH was adjusted to 6.5 using NaOH (0.1 M). Complexation was found to be complete once less than 5% free ligand was detected, as determined by LCMS (method A).

Tri-tert-butyl 2,2′,2″-(10-(2-((((9H-fluoren-9-yl)methoxy)carbonyl)amino)-3-((2-methoxy-2-oxoethyl)amino)-3-oxopropyl)-1,4,7,10-tetraazacyclododecane-1,4,7-triyl)triacetate (2). ¹H-NMR (CDCl₃, 500 MHz, ppm): 8.75(s (br), NH), 7.75 (d, 2H), 7.61 (m, 2H), 7.41-7.28 (m, 4H), 4.77 (s(br), 1H), 4.41-3.05 (m, 31H), 1.43-1.39 (m, 27H). ¹³C-NMR (CDCl₃, 125 MHz, ppm): 169.9, 169.3, 161.3, 161.0, 143.7, 143.5, 141.3, 141.2, 127.8, 127.1, 125.2, 119.9, 117.4, 115.0, 113.1, 82.6, 67.5, 54.8, 52.2, 50.9, 46.9, 41.1, 29.7, 28.0. LC-ESI-MS: calcd. for C₄₇H₇₁N₆O₁₁: 895.5. Found: 895.5 [M+H]⁺, Rₜ=6.36 min.

Tri-tert-butyl 2,2′,2″-(10-(2-amino-3-((2-methoxy-2-oxoethyl)amino)-3-oxopropyl)-1,4,7,10-tetraazacyclododecane-1,4,7-triyl)triacetate (3). ¹H-NMR (D₂O, 500 MHz, ppm): 3.95 (d, 2H), 3.63 (s, 3H), 3.48-2.2 (m, 24H), 1.42-1.34 (m, 27H). LC-ESI-MS: calcd. for C₃₂H₆₁N₆O₉: 673.5 Found: 673.5 [M+H]⁺, Rₜ=5.02 min.

2,2′,2″-(10-(2-amino-3-((2-methoxy-2-oxoethyl)amino)-3-oxopropyl)-1,4,7,10-tetraazacyclododecane-1,4,7-triyl)triacetic acid (4). ¹H-NMR (D₂O, 500 MHz, ppm): 3.64-3.63 (m, 5H), 3.51 (s, 3H), 3.51-2.58 (m, 22H). ¹³C-NMR (D₂O, 125 MHz, ppm): 175.8, 171.6, 170.4, 170.3, 58.1, 56.7, 53.9, 52.9, 52.4, 50.4, 48.9, 46.0, 41.22. LC-ESI-MS: calcd. for C₂₀H₃₇N₆O₉: 505.25 Found: 505.3 [M+H]⁺, R=6.1 min (method B, ultra-aqueous column).

Tri-tert-butyl 2,2′,2″-(10-(2-(2-((((9H-fluoren-9-yl)methoxy)carbonyl)amino)-3-(4-hydroxy-3,5-diiodophenyl)propanamido)-3-(((S)-1-methoxy-1-oxopropan-2-yl)amino)-3-oxopropyl)-1,4,7,10-tetraazacyclododecane-1,4,7-triyl)triacetate (5). ¹H-NMR (CDCl₃, 500 MHz, ppm): 7.73 (d, 2H), 7.61-7.54 (m, 4H), 7.41-7.28 (m, 4H), 4.95 (s(br), 1H), 4.39-2.94 (m, 31H), 1.47-1.41 (m, 27H). ¹³C-NMR (CDCl₃, 125 MHz, ppm): 169.9, 161.5, 161.2, 156.6, 152.7, 143.7, 141.2, 141.1, 140.0, 139.9, 127.7, 127.2, 125.4, 125.3, 119.8, 117.4, 115.0, 82.8, 82.4, 67.5, 58.4, 56.9, 55.1, 52.2, 49.3, 48.6, 46.9 41.3, 28.0, 27.9. LC-ESI-MS: calcd. for C₅₆H₇₈I₂N₇O₁₃: 1310.4. Found: 1310.3 [M+H]⁺, R=7.2 min.

Tri-tert-butyl 2,2′,2″-(10-(2-(2-((((9H-fluoren-9-yl)methoxy)carbonyl)amino)-3-(4-hydroxy-3,5-diiodophenyl)propanamido)-3-(((S)-1-methoxy-1-oxopropan-2-yl)amino)-3-oxopropyl)-1,4,7,10-tetraazacyclododecane-1,4,7-triyl)triacetate (6). ¹H-NMR (CDCl₃, 500 MHz, ppm): 7.59 (s, 2H), 7.41-7.28 (m, 4H), 4.95 (m, 2H), 3.9-2.94 (m, 32H), 1.50-1.44 (m, 27H). LC-ESI-MS: calcd. for C₄₁H₆₈I₂N₇O₁₁. 1088.3 Found: 1088.3 [M+H]⁺, Rₜ=5.2 min.

2,2′,2″-(10-(2-(2-amino-3-(4-hydroxy-3,5-diiodophenyl)propanamido)-3-((2-methoxy-2-oxoethyl)amino)-3-oxopropyl)-1,4,7,10-tetraazacyclododecane-1,4,7-triyl)triacetic acid (7). ¹H-NMR (D₂O, 500 MHz, ppm): 7.69 (s, 2H), 4.62 (d, 1H), 4.18 (s, 1H), 3.94-2.80 (m, 30H). ¹³C-NMR (D₂O, 125 MHz, ppm): 171.0, 170.6, 170.1, 160.8, 160.5, 154.9, 140.8, 140.3, 129.4, 117.5, 115.1, 84.4, 84.0, 61.9, 56.1, 53.8, 53.1, 51.5, 51.3, 40.9. LC-ESI-MS: calcd. for C₂₉H₄₄I₂N₇O₁₁: 920.1 Found: 920.1 [M+H]⁺, Rₜ=3.9 min.

Tri-tert-butyl 2,2′,2″-(10-(2-((((9H-fluoren-9-yl)methoxy)carbonyl)amino)-3-oxo-3-(((R)-1-phenylethyl)amino)propyl)-1,4,7,10-tetraazacyclododecane-1,4,7-triyl)triacetate (8). ¹H-NMR (CDCl₃, 500 MHz, ppm): 8.33 (s, 1H), 8.25 (s, 1H), 7.74 (m, 2H), 7.63-7.59 (m, 2H), 7.41-7.16 (m, 9H), 5.01 (m, 1H), 4.69(s, 1 H), 4.39-2.21 (m, 3H), 3.72-2.92 (m, 25H) 1.47-1.42 (m, 31H). ¹³C-NMR (CDCl₃, 125 MHz, ppm): 169.4, 161.3, 161.0, 143.5, 141.3, 128.6, 128.5, 127.7, 127.2, 127.1, 126.2, 126.1, 125.2, 119.9, 117.4, 82.6, 55.1, 53.4, 51.0, 49.9, 49.8, 46.9, 28.9, 22.0. LC-ESI-MS: calcd. for C₅₂H₇₅N₆O₉: 927.5 Found: 927.4 [M+H]⁺, Rₜ=6.76 min.

Tri-tert-butyl 2,2′,2″-(10-(2-amino-3-oxo-3-(((R)-1-phenylethyl)amino)propyl)-1,4,7,10-tetraazacyclododecane-1,4,7-triyl)triacetate (9). ¹H-NMR (D₂O, 500 MHz, ppm): 7.26-7.21 (m, 5H), 4.62 (d, 1H), 4.81 (s, 1H), 3.41-2.80 (m, 26H), 1.35-1.25 (m,31H LC-ESI-MS: calcd. for C₃₇H₆₅N₆O₇: 705.5 Found: 705.4 [M+H]⁺, Rₜ=5.5/5.7 min.

2,2′,2″-(10-(2-amino-3-oxo-3-(((R)-1-phenylethyl)amino)propyl)-1,4,7,10-tetraazacyclododecane-1,4,7-triyl)triacetic acid (10). ¹H-NMR (D₂O, 500 MHz, ppm): 7.45-7.37 (m, 5H), 4.98-4.96 (m, 1H), 3.51 (m, 1H), 3.19-2.26 (m, 23H), 1.51 (t, 3H). ¹³C-NMR (D₂O, 125 MHz, ppm): 179.0, 176.6, 128.8, 128.7, 126.0, 125.8, 59.3, 49.4, 21.1. LC-ESI-MS: calcd. for C₂₅H₄₁N₆O₇: 537.3 Found: 537.3 [M+H]⁺, Rₜ=1.06/ 1.21 min.

Tri-tert-butyl 2,2′,2″-(10-(2-(2-((((9H-fluoren-9-yl)methoxy)carbonyl)amino)-3-(4-hydroxy-3,5-diiodophenyl)propanamido)-3-oxo-3-(((R)-1-phenylethyl)amino) propyl)-1,4,7,10-tetraazacyclododecane-1,4,7-triyl)triacetate (11). ¹H-NMR (CDCl₃, 500 MHz, ppm): 7.71-7.18 (m, 15H), 4.41-3.95 (s(br), 1H), 4.81 (m, 1 h) 4.39-2.74 (m, 29H), 1.51-1.25 (m, 31H). ¹³C-NMR (CDCl₃, 125 MHz, ppm): 172.0, 171.0, 169.9, 161.4, 155.8, 152.9, 143.8, 141.1, 140.0, 139.8, 128.5, 127.7, 127.6, 127.2, 125.9, 125.2, 125.1, 120.0, 119.9, 119.8, 83.2, 82.0, 67.3, 56.9-46.9 (9 broad peaks), 28.0, 22.3. LC-ESI-MS: calcd. for C₆₁H₈₂I₂N₇O₁₁: 1342.4. Found: 1342.3 [M+H]⁺, Rₜ=7.44 min.

Tri-tert-butyl2,2′,2″-(10-(2-(2-amino-3-(4-hydroxy-3,5-diiodophenyl)propanamido)-3-oxo-3-(((R)-1-phenylethyl)amino)propyl)-1,4,7,10-tetraazacyclododecane-1,4,7-triyl)triacetate (12). ¹H-NMR (CD₃OD, 500 MHz, ppm): 7.64 (d, 2H), 7.27-7.15 (m, 5H), 5.02 (d, 1 H), 4.10-2.85 (m, 28H), 1.51-1.25 (m, 30H). LC-ESI-MS: calcd. for C₄₆H₇₂I₂N₇O₉: 1120.3 Found: 1120.2 [M+H]⁺, Rₜ=5.6/5.8 min.

2,2′,2″-(10-(2-(2-amino-3-(4-hydroxy-3,5-diiodophenyl)propanamido)-3-oxo-3-(((R)-1-phenylethyl)amino)propyl)-1,4,7,10-tetraazacyclododecane-1,4,7-triyl)triacetic acid (13). ¹H-NMR (CD₃OD, 500 MHz, ppm): 8.38 (d, 1H) 7.65 (d, 2H), 7.33-7.24 (m, 5H), 4.98 (d, 1H), 4.76-4.03 (m, 5H), 3.64-2.50 (m, 18H), 1.51-1.39 (t, 3H). ¹H-NMR (CD₃OD, 125 MHz, ppm): 169.7, 169.2, 160.2, 159.9, 154.9, 142.9, 140.6, 129.7, 128.1, 126.8, 125.9, 117.4, 114.8, 89.4, 81.2, 81.1, 53.8, 53.3, 51.3, 50.9, 49.0, 48.2, 34.3. LC-ESI-MS: calcd. for C₃₄H₄₈I₂N₇O₉: 952.2 Found: 952.2 [M+H]⁺, Rₜ=4.8/ 4.9 min.

2,2′,2″-(10-(2-acetamido-3-oxo-3-(((R)-1-phenylethyl)amino)propyl)-1,4,7,10-tetraazacyclododecane-1,4,7-triyl)triacetic acid (14). tri-tert-butyl 2,2′,2″-(10-(2-amino-3-oxo-3-(((R)-1-phenylethyl)amino)propyl)-1,4,7,10-tetraazacyclododecane-1,4,7-triyl)triacetate (9, 11 mg, 0.015 mmol), was dissolved in dichloromethane (2 mL). Triethylamine (2 eq., 5 μL), followed by acetic anhydride (5 eq.) was added and the reaction was stirred over night at room temperature. LCMS analysis indicated complete conversion of starting material. The product was purified using method A. The fractions containing product were pooled and lyophilized to afford the protected precursor (1 mg, 0.0015 mmol, 10% yield after purification). Subsequent deprotection of the tert-butyl acetate moieties yielded the final product 14. ¹H-NMR (CD₃OD, 500 MHz, ppm): 8.4 (s, 1H) 7.29-7.15 (m, 5H), 4.95 (s, 1 H), 4.01-2.85 (m, 24H), 1.91 (s, 3H), 1.39 (t, 3H). ¹H-NMR (CD₃OD, 125 MHz, ppm): 178.3, 154.4, 147.3, 143.2, 128.2, 126.8, 125.9, 56.0, 55.7, 53.3, 50.6, 49.1, 48.4, 29.3, 15.6. LC-ESI-MS: calcd. for C₂₇H₄₃N₆O₈: 579.3 Found: 579.3 [M+H]⁺, Rₜ=1.7 min.

(S)-tert-butyl 2-amino-3-(4-hydroxy-3,5-diiodophenyl)propanoate (15). (S)-2-((((9H-fluoren-9-yl)methoxy)carbonyl)amino)-3-(4-hydroxy-3,5diiodophenyl)propanoic acid (0.67g, 1 mmol) was dissolved in tert-butyl acetate (7 mL). Perchloric acid (1.5 eq, 90 μL) was added and the solution was stirred at room temperature over night. The acid was neutralized with a saturated aqueous solution of NaHCO₃ and the product was extracted three times with dichloromethane. The organic layer was collected, dried with Na₂SO₄ and concentrated. The crude product was subsequently purified with a 0-20% gradient of EtOAc in Hexanes on a silica combiflash column. The intermediate product is isolated as yellow oil, which solidifies upon standing. This intermediate was taken up in DMF and the Fmoc protective group was removed according to general procedure 2. The deprotected synthon (15) was separated by filtration of the solid support beads and added to the subsequent reaction without further purification steps.

Tri-tert-butyl 2,2′,2″-(10-(2-((((9H-fluoren-9-yl)methoxy)carbonyl)amino)-3-(((S)-1-(tert-butoxy)-3-(4-hydroxy-3,5-diiodophenyl)-1-oxopropan-2-yl)amino)-3-oxopropyl)-1,4,7,10-tetraazacyclododecane-1,4,7-triyl)triacetate (16). ¹H-NMR (CDCl₃, 500 MHz, ppm): 7.71-7.26 (m, 10H), 4.47-2.90 (m, 29H), 1.48-1.25 (m, 27H). ¹³C-NMR (CDCl₃, 125 MHz, ppm): 169.9, 160.7, 160.5, 152.6, 152.6, 143.4, 141.3, 141.2, 139.9, 129.7, 128.3, 127.8, 127.1, 127.1, 125.2, 124.9, 120.1, 116.5, 114.2, 83.2, 82.0, 67.5, 67.3, 55.1, 54.5, 54.1, 46.9, 46.9, 46.7, 27.8. LC-ESI-MS: calcd. for C₅₇H₈₁I₂N₆O₁₂: 1295.4. Found: 1295.4 [M+H]⁺, Rₜ=7.9 min.

Tri-tert-butyl 2,2′,2″-(10-(3-(((S)-1-(tert-butoxy)-3-(4-hydroxy-3,5-diiodophenyl)-1-oxopropan-2-yl)amino)-3-oxo-2-((R)-2-phenylpropanamido)propyl)-1,4,7,10-tetraazacyclododecane-1,4,7-triyl)triacetate (18). ¹H-NMR (CDCl₃, 500 MHz, ppm): 8.07 (d, 1H), 7.53-7.26 (m, 7H), 4.86 (m, 1H), 4.78 (m, 1H) 4.47-2.90 (m, 26H), 1.48-1.25 (m, 30H). LC-ESI-MS: calcd. for C₅₁H₇₉I₂N₆O₁₁: 1205.4. Found: 1205.3 [M+H]⁺, Rₜ=7.3 min.

2,2′,2″-(10-(3-(((S)-1-carboxy-2-(4-hydroxy-3,5-diiodophenyl)ethyl)amino)-3-oxo-2-((R)-2-phenylpropanamido)propyl)-1,4,7,10-tetraazacyclododecane-1,4,7-triyl)triacetic acid (19). ¹H-NMR (CDOD₃, 500 MHz, ppm): 7.97 (d, 1H), 7.61-7.28 (m, 7H), 4.66 (m, 1H), 4.02-2.61 (m, 26H), 1.59 (m, 3H). LC-ESI-MS: calcd. for C₃₅H₄₇I₂N₆O₁₁: 981.1. Found: 981.1 [M+H]⁺, Rₜ=5.4 min.

2,2′,2″-(10-(3-(((1-Carboxy-2-(4-hydroxy-3,5-diiodophenyl)ethyl)-amino)-2-(2-(4-isobutylphenyl)propanamido)-3-oxopropyl)-1,4,7,10-tetraazacyclododecane-1,4,7-triyl)triacetic Acid, (8). ¹H NMR (D₂O, 500 MHz, ppm): 7.52 (d, 2H), 7.16-7.00 (m, 4H), 4.48 (m, 1H), 3.86-2.85 (m, 22H), 1.78 (m, 2H), 1.78 (m, 1H), 1.21 (m, 3H) 0.81 (m, 6H). ¹³C NMR (D2O, 125 MHz, ppm): 171.0, 170.6, 161.2, 153.2, 142.1, 140.6, 139.9, 129.3, 128.8, 128.1, 126.8, 83.9, 65.1, 53.4, 34.6, 30.0, 29.4, 27.4, 24.7, 22.3, 21.5, 21.3. LC-ESI-MS calcd for C₃₉H₅₅I₂N₆O₁₁: 1037.2. Found: 1037.1 [M +H]⁺. Looking at FIG. 21A, X═CO-Ibu and Y=Tyr(I)₂—OH for (8).

2,2′,2″-(10-(2-Carboxy-2-(2-(4-isobutylphenyl)propanamido)-ethyl)-1,4,7,10-tetraazacyclododecane-1,4,7-triyl)triacetic Acid, (9). ¹H NMR (D₂O, 500 MHz, ppm): 7.26 (d, 2H), 7.20 (d,2H), 3.73-2.83 (m, 26H), 2.43 (m, 2H), 1.78 (t, 1H), 1.41 (d, 3H),0.81 (m, 6H). ¹³C NMR (D₂O, 125 MHz, ppm): 177.5, 176.9, 141.9, 138.5, 130.1, 127.3, 70.2, 54.4, 46.5, 44.8, 30.7, 27.8, 22.8. LC-ESI-MS calcd for C₃₀H₄₈N₅O₉: 622.3. Found: 622.4 [M +H]⁺. Looking at FIG. 21A, X═CO-Ibu and Y═H for (9).

### Gd-Complexes

TABLE 6
Summary of fraction of compound (0.1 mM) bound to 4.5% (w/v)
HSA and the relaxivity measured in the absence and presence
of HSA (absence/presence) at two frequencies (60 and 20 MHz).

| | | r1 (mM⁻¹s⁻¹, | r1 (mM⁻¹s⁻¹, |
|---|---|---|---|
| Gd complex | % HSA binding | 60 MHz) | 20 MHz) |
| Gd (4) | 7% | 4.1/7.6 | 4.5/8.4 |
| Gd (7) | 6% | 3.9/6.9 | 4.7/7.05 |
| Gd (8) | 90% | 6.7/21.1 | 7.1/31.8 |
| Gd (9) | 70% | 4.8/22.4 | 5.6/37.2 |
| Gd (10) | 21% | 6.3/16.7 | 6.9/20.9 |
| Gd (13-R) | 36% | 6.4/11.4 | 6.6/18.4 |
| Gd (13-S) | 39% | 6.1/10.9 | 7.1/18.5 |
| Gd (14) | 21% | 4.5/6.3 | 5.2/8.6 |
| Gd (19) | 69% | 6.4/21.8 | 6.0/30.7 |

### Example 4

This example discloses experimental data for ⁶⁴CuDOTAla.

GdL1 was dissolved in a pH 3 citrate (50 mM) to afford a 0.1 mM solution and incubated for 14 days at 37° C. Within this time, slow transchelation of GdL1 to Gd(citrate) took place. This solution was then incubated with ⁶⁴CuCl₂ (0.2 mCi) at room temperature and the pH was increased to 9 using 0.1 M NaOH solution and the solution was analysed using HPLC (Method C: Phenomenex C18 column, 150×4.6 mm, 5 mycron; Solvent A: H₂O with 0.1% TFA, solvent B: Acetonitrile with 0.1% TFA. Gradient: 0-80% B in 12 min, flow: 1 mL/min). Analysis indicated re-complexation of Gd, as well as incorporation of 80% of the free ⁶⁴Cu into the chelate. Rᵣ ⁶⁴CuL1 : 4.3 min.

### Example 5

The heptadentate ligand, CyPic3A, was designed to chelate Gd(III) in a stable fashion while allowing for a hydration state of q>1 (FIG. 26, highlighted). [Gd(CyPic3A)(H₂O)₂]⁻ exhibits high relaxivity and maintains stability and inertness comparable to clinically used probes. The synthesis and evaluation of the physical properties of [Gd(CyPic3A)(H₂O)₂]⁻ are discussed below.

CyPic3A possesses stabilizing structural factors that compensate for reduced denticity. Trans-1,2-diaminocyclohexane was incorporated into the ligand framework. Exchanging 1,2-diaminoethane for this rigidified linkage has been shown to increase the stability and inertness of lanthanide(III) complexes significantly.^{17,18} The bidentate 2-picolinate functionality is similarly stabilizing.^{19,20} These two moieties were fused to comprise the ligand backbone.

Coupling of mono-BOC protected trans-1,2-diaminocyclohexane (XIV)²¹ and methyl 6-formylpyridine-2-carboxylate (XII)²² (prepared in one and two steps, respectively) via reductive amination produced the BOC-protected backbone XV in high yield. Deprotection of XV followed by alkylation of the two amines afforded O-protected CyPic3A (XVII). O-deprotection yielded the ligand, isolated as CyPic3A•2TFA. In total, this straightforward synthesis required seven steps, each affording high yields and few requiring chromatography (see below). Complex formation occurred rapidly in water upon addition of GdCl₃.6H₂O followed by adjustment of the pH to 6.5. It is important to note that 2,6-pyridinedicarboxylate, from which XII is derived, is a highly modular synthon.^{23,24} Thus, it is envisioned that bifunctional analogues of CyPic3A can be prepared.

The bis(aqua) hydration state of [Gd(CyPic3A)(H₂O)₂]⁻ was confirmed by analogy through luminescence lifetime experiments on the Eu(III) congener measured in H₂O vs. D₂O.^{25,26} Following excitation at 396 nm, the emission intensity at 616 nm was monitored with respect to time to determine the first order rate constant. There is an empirical relationship that relates the difference in luminescence decay rates measured in H₂O and D₂O to the hydration number.²⁷ It was determined that q=2.09 using this method.

The mean residency time of the coordinated H₂O molecules was also measured by recording the ¹⁷O transverse relaxation (T₂) times of solvent H₂O between 278 and 363 K in the presence and absence of [Gd(CyPic3A)(H₂O)₂]⁻. The reduced relaxation rate (1/T₂ᵣ) is this relaxation rate difference normalized to the mole fraction of water coordinated to the Gd(III). A four-parameter fit to this data yielded water exchange kinetic parameters and an estimate of the electronic relaxation time (T₁ₑ).^{6,28,29} The water residency time at 310 K)(Tₘ³¹⁰) was determined to be 14±1 ns, and this very short residency time is optimal for relaxivity applications.

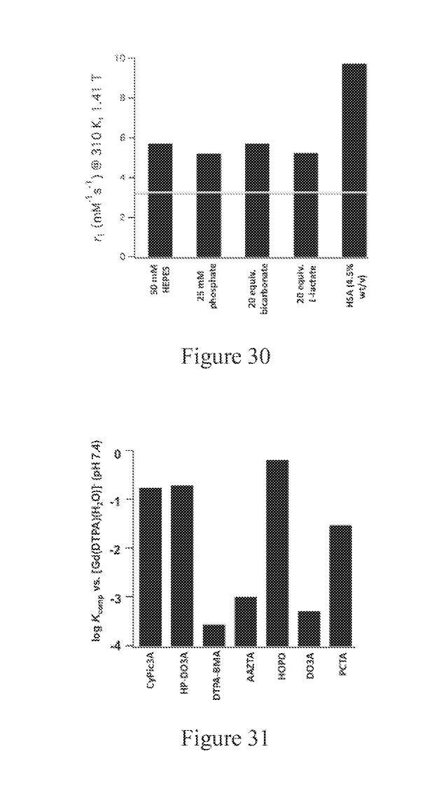

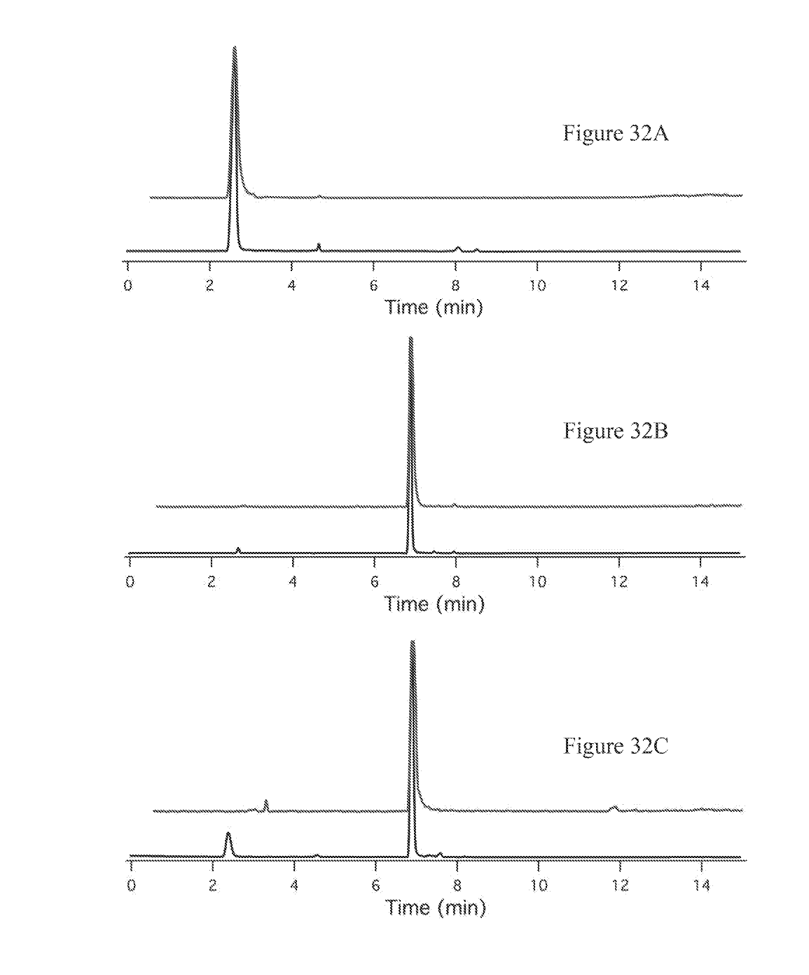

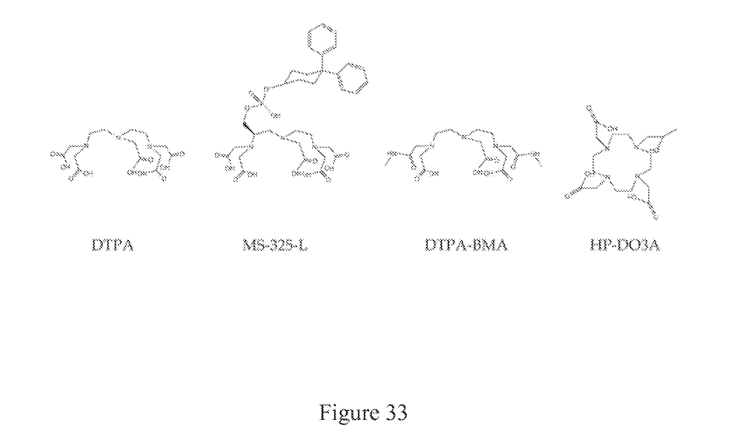

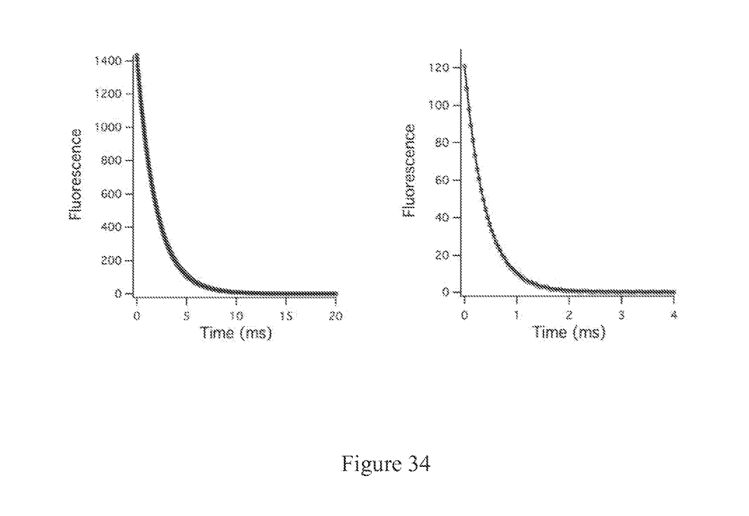

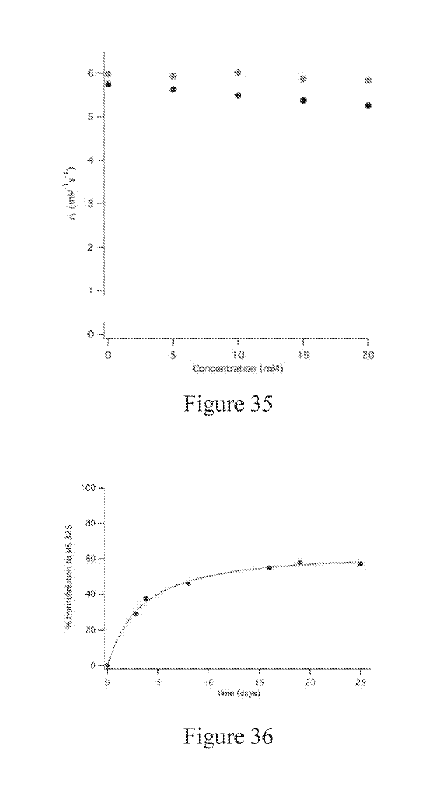

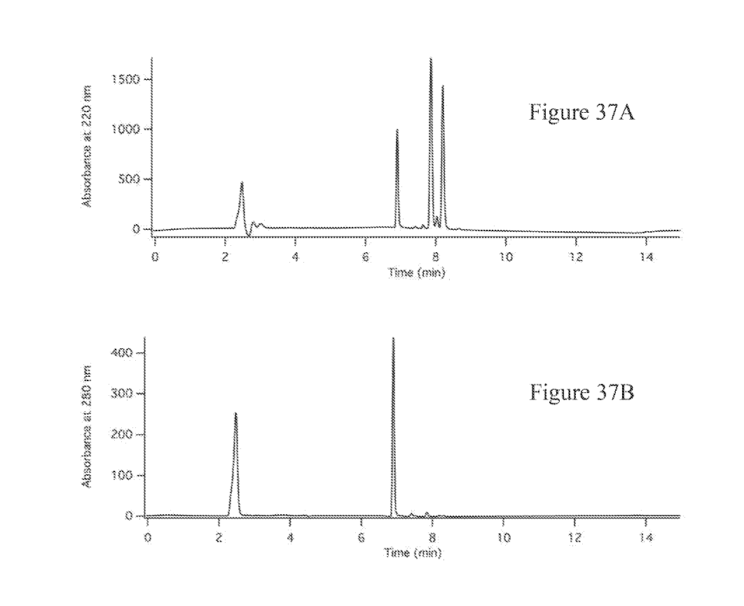

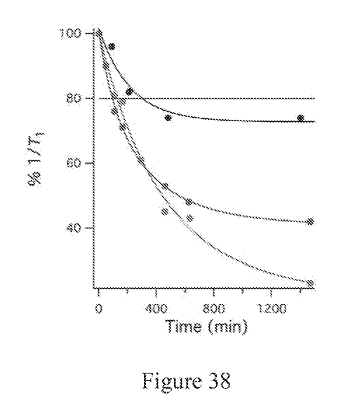

The relaxivity of [Gd(CyPic3A)(H₂O)₂]⁻ is consistent with two inner-sphere water ligands. At 310 K in pH 7.4 50 mM HEPES buffer, r₁=5.70 mM⁻¹ s⁻¹ at 1.41 T (7.53 mM⁻¹s⁻¹ at 298 K). This is 75% higher than [Gd(DTPA)(H₂O)]²⁻ measured under these conditions (see below for detailed table of r₁ values). To probe the effects of potentially coordinating anions, nwas measured in the presence of 20 equiv. carbonate or L-lactate and also in 25 mM phosphate buffer (pH 7.4). The results are summarized in FIG. 30. The r₁ values across these conditions indicate that the water coordination sites remain resistant to endogenously encountered bidentate anions known to significantly decrease the r₁ of q=2 Gd(III) complexes such as Gd(DO3A).³⁰ Relaxivity was also measured to be 8.90 mM⁻¹ s⁻¹ in bovine blood plasma at 310 K, and a value of 9.73 mM⁻¹ s⁻¹ was recorded in the presence of 4.5% w/v human serum albumin (HSA) at 1.41 T (12.03 mM⁻¹ s⁻¹ at 0.47 T). Separation of free and HSA-bound [Gd(CyPic3A)(H₂O)₂]⁻ by ultrafiltration (5,000 Da cut-off) followed by quantification of filtrate Gd content by ICP-MS revealed 8% of [Gd(CyPic3A)(H₂O)₂]⁻ associated with HSA. The large r₁ boost provided by the 8% protein bound complex suggests that an r₁ of 51 and 71 mM⁻¹ s⁻¹ at 1.41 and 0.47 T, respectively, could potentially be achieved through full macromolecular association.

As a means of assessing thermodynamic stability relative to other Gd(III)-complexes, [Gd(CyPic3A)(H₂O)₂]⁻ was challenged with DTPA in pH 7.4 25 mM Tris buffer. CyPic3A possesses a strong chromophore at 280 nm and the relative distributions of complex and free ligand are easily monitored by LC-MS. From this information it was possible to deduce equilibrium constants (K_{comp}) for the competition reaction described in Equation 5,³¹ where L=DTPA and L′=challenging chelator. A K_{comp} of 0.17 at was obtained at pH 7.4. This indicates that the affinity of CyPic3A for Gd(III) is about 5-fold lower than DTPA at pH 7.4. FIG. 31 compares this result to calculated K_{comp} values for ligands (L′) of complexes used clinically in MRI³¹⁻³³ and previously characterized q=2 Gd(III) complexes.^{32,34-36} The complex [Gd(CyPic3A)(H₂O)₂]⁻ is of comparable stability to [Gd(HP-DO3A)(H₂O)] (ProHance®) and >600-fold more stable than [Gd(DTPA-BMA)(H₂O)] (Omniscan®). CyPic3A also formed more stable complexes than many of the q=2 complexes (See below for detailed table and comparison against the angiography probe MS-325). Thus, it appears that structural factors considered in the design of CyPic3A compensate for the stability penalty incurred by reduction of available donors.

$$[ GdL ] + {L}^{′} ↔ [ {GdL}^{′} ] + L ; \\ {K}_{comp} = \frac{[ GdL ] [ {L}^{′} ]}{[ {GdL}^{′} ] [ L ]} \qquad ( Eq . 5 )$$

Kinetic inertness was also taken into account in the evaluation of potential probes. In this regard, [Gd(CyPic3A)(H₂O)₂]⁻ was challenged with 1 equiv. [ZnPO₄]⁻ as a slurry in pH 7.0 phosphate buffer at 310 K (see below). Several clinically utilized Gd(III) complexes have been benchmarked relative to one another in this manner.³⁷ As Gd(III) replaced by Zn(II) precipitates from the solution as GdPO₄, the relaxation rate 1/T₁ decreases. In this method, the time required to reach 80% of the initial 1/T₁ value was utilized as an index of kinetic inertness. A time to 80% of the initial 1/T₁ of 95 min was measured for [Gd(CyPic3A)(H₂O)]⁻, 124 minutes for [Gd(DTPA-BMA)(H₂O)] and 383 minutes for [Gd(DTPA)(H₂O)]²⁻. As with the thermodynamic stability of [Gd(CyPic3A)(H₂O)₂]⁻ at pH 7.4, the observed kinetic stability also compared well to clinically used agents.

### References for Example 5

1 P. Caravan, J. J. Ellison, T. J. McMurry and R. B. Lauffer. Chem. Rev. 1999, 99, 2293.
2 R. B. Lauffer. Chem. Rev. 1987, 87, 901.
3 P. Caravan. Chem. Soc. Rev. 2006, 35, 512.
4 L. Helm and A. E. Merbach. Chem. Rev. 2005, 105, 1923.
5 S. Dumas, V. Jacques, W.-C. Sun, J. S. Troughton, J. T. Welch, J. M. Chasse, H. Willich-Schmitt and P. Caravan. Invest. Radiol. 2010, 45, 600.
6 P. Caravan, C. T. Farrar, L. Frullano, and R. Uppal, Contrast Media. Mol. Imag. 2009, 89.
7 E. Boros, M. Polasek, Z. Zhang and P. Caravan. J. Am. Chem. Soc. 2012, 134, 19858.
8 D. J. Mastarone, V. S. R. Harrison, A. L. Eckermann, G. Parigi, C. Luchinat, and T. J. Meade. J. Am. Chem. Soc. 2011, 133, 5329.
9 P. Caravan. Acc. Chem. Res. 2009, 42, 851.
10 R. Strauch, D. J. Mastarone, P. A. Sukerkar, Y. Song, J. J. Ipsaro and T. J. Meade. J. Am. Chem. Soc. 2011, 133, 16436.
11 E. Boros and P. Caravan. J. Med. Chem. 2013, 56, 1782.
12 S. Aime, L. Calabi, C. Cavallotti, E. Gianolio, G. B. Giovenzana, P. Losi, A. Maiocchi, G. Palmisano and M. Sisti. Inorg. Chem. 2004, 43, 7588.
13 A. Datta and K. N. Raymond. Acc. Chem. Res 2009, 42, 938.
14 S. Aime, M. Botta, L. Frullano, S. G. Crich, G. Giovenzana, R. Pagliarin, G. Palmisano, F. R. Sirtori and M. Sisti. J. Med. Chem. 2000, 43, 4017.
15 S. Aime, M. Botta, S. G. Crich, G. Giovenzana, G. Jommi, R. Pagliarin and M. Sisti. Inorg. Chem. 1997, 36, 2992.
16 S. Aime, M. Botta, S. G. Crich, G. Giovenzana, R Pagliarin, M. Sisti and E. Terreno. Magn. Reson. Chem. 1998, 36, S200.
17 T. J. McMurry, C. G. Pippin, C. Wu, K. A. Deal, M. W. Brechbiel, S. Mirzadeh and O. A. Gansow. J. Med. Chem. 1998, 41, 3546.
18 P. Thakur, J. L. Conca, L. J. Van de Burgt and G. R. Choppin. J. Coord. Chem. 2009, 62,3719.
19 A. M. Nonat, C. Gateau, P. H. Fries, L. Helm and M. Mazzanti. Eur. J. Inorg. Chem. 2012, 51, 2049.
20 E. Balogh, M. Mato-Iglesias, C. Platas-Iglesias, E. Toth, K. Djanashvili, J. A. Peters, A. de Blas and T. Rodriguez-Blas. Inorg. Chem. 2006, 45, 8719.
21 P. D. Garimella, A. Datta, D. W. Romanini, K. N. Raymond and M. B. Francis. J. Am. Chem. Soc. 2011, 133, 14704.
22 C. Platas-Iglesias, M. Mato-Iglesias, K. Djanashvili, R. N. Muller, L. Vander Elst, J. A. Peters, A. de Blas and T. Rodriguez-Blas. Chem. Eur. J. 2004, 10, 3579.
23 L. Syper, K. Kloc and J. MLochowski 1Tetrahedron 1980, 36, 123.
24 M. Kotera, J.-M. Lehn and J.-P. Vigneron. Tetrahedron 1995, 51, 1953.
25 W. D. Horrocks and D. R. Sudnick. Acc. Chem. Res. 1981, 14, 342.
26 L. M. Manus, R. C. Strauch, A. H. Hung, A. L. Eckermann and T. J. Meade. Anal. Chem. 2012, 84, 6278.
27 A. Beeby, I. M. Clarkson, R. S. Dickins, S. Faulkner, D. Parker, L. Royle, A. S. de Sousa, J. A. G. Williams and M. J. Woods. Chem. Soc. Perkin Trans. 2 1999, 493.
28 P. Caravan, G. Parigi, J. M. Chasse, N. J. Cloutier, J. J. Ellison, R. B. Lauffer, C. Luchinat, S. A. McDermid, M. Spiller and T. J. McMurry. Inorg. Chem. 2007, 46, 6632.
29 T₁ₑ³¹⁰, the enthalpy of activation of the water exchange reaction (IP) and the activation energy for perturbations to the electronic ground state (E_{T1e}) are 179±109 ns, 29.1±1.5 kJ/mol and 35.1±13.3 kJ/mol, respectively.
30 M. Polasek and P. Caravan. Inorg. Chem. 2013, 52, 4084.
31 P. Caravan, C. Comuzzi, W. Crooks, T. J. McMurry, G. R. Choppin and S. R. Woulfe. Inorg. Chem. 2001, 40, 2170.
32 K. Kumar, C. A. Chang, L. Francesconi, D. D. Dischino, M. F. Malley, J. Z. Gougoutas and M. F. Tweedle. Inorg. Chem. 1994, 33, 3567.
33 L. Moriggi, C. Cannizzo, C. Prestinari, F. Berriere and L. Helm. Inorg. Chem. 2008, 47, 8357.
34 Z. Baranyai, F. Uggeri, G. B. Giovenzana, A. Benyei, E. Bracher and S. Aime. Chem. Eur. J. 2009, 15, 1696.
35 D. M. J. Doble, M. Melchior, B. O'Sullivan, C. Siering, J. Xu, V. C. Pierrre and K. N. Raymond. Inorg. Chem. 2003, 42, 4930.
36 R. Delgado, S. Qunitino, M. Teixeira and A. Zhang, A. J. Chem. Soc. Dalton Trans. 1997, 55.
37 S. Laurent, L. Vander Elst, C. Henoumon, and R. N. Muller. Contrast Media. Mol. Imag. 2010, 5, 305.

### Example 5

### Experimental Section

### General Methods and Materials

All chemicals and solvents were purchased commercially and used without further purification. NMR spectra were recorded on either a 500 MHz or 400 MHz Varian spectrometer equipped with a 5 mm broadband probe. Chemical shifts are reported in 6 (ppm). For¹H NMR spectra, the peak from residual protio solvent was used as an internal reference.¹ For ¹³C NMR spectra, the solvent peaks were used as an internal reference, except when acquired in D₂O, in which case dioxane was used as the internal reference.¹ Liquid chromatography-electrospray mass spectrometry (LC-MS) was performed using an Agilent 1100 Series apparatus with an LC/MSD trap and Daly conversion diode detector with UV detection at 220, 254 and 280 nm. Characterization of CyPic3A, [Gd(CyPic3A)(H₂O)₂]⁻, [Eu(CyPic3A)(H₂O)₂]⁻ and ligand competition experiments were performed using a Kromasil C18 reversed-phase column (250 mm×4.6 mm) by the following method: the mobile phase was a mixture of 10 mM aqueous ammonium formate (eluent A) and a solution of acetonitrile/ 10% 10 mM aqueous ammonium formate (eluent B). Starting from 5% B, the fraction of B increased to 95% over 12 minutes. The column was washed with 95% B for 2 minutes and than ramped to 5% B. The system was re-equilibrated at 5% B. Reversed-phase semi-preparative purification was performed on the Rainin Dynamax HPLC system with UV detection at 254 nm using a Kromasil C4 (250×21.8 cm) column. The method used for purification is as follows: the mobile phase was a mixture of water (eluent A) and acetonitrile (eluent B), each containing 0.1% TFA. Starting from 5% B, the fraction of B increased to 25% over 23 minutes. The column was washed with 95% B for 2 minutes and then ramped to 5% B. The system was re-equilibrated at 5% B. Gadolinium concentrations were determined using an Agilent 7500a ICP-MS system. All samples were diluted with 0.1% Triton X-100 in 5% nitric acid containing 20 ppb of Lu (as internal standard). The ratio of Gd (157.25)/Lu (174.97) was used to quantify the Gd concentration. A linear calibration curve ranging from 0.1 ppb to 200 ppb was generated daily for the quantification. pH was measured using a ThermoOrion pH meter connected to a VWR Symphony glass electrode. Ultrafiltration was performed using Ultrafree-MC Microcentrifuge filters with a 5,000 Da cut-off PLCC cellulosic membrane. Incubation of samples was performed using a New Brunswick Scientific Inova4000 Incubator Shaker.

### Relaxometry

Relaxivity measurements were performed on a Bruker mq60 or Bruker mq20 Minispec at 1.41 T and 0.47 T, respectively, and 37° C. Longitudinal (T₁) relaxation was acquired via an inversion recovery experiment on 10 inversions of duration ranging between 0.05×T₁ and 5×T₁. Relaxivity (r₁) was determined from the slope of a plot of 1/T₁ vs. [Gd] for at least 4 concentrations of Gd(III). The transverse (T₂) relaxation times of ¹⁷O were acquired at 500 MHz using a CPMG pulse sequence at temperatures ranging from 278 to 368 K. Reduced relaxation rates (1/T₂ᵣ) were calculated by dividing the [Gd(CyPic3A)(H₂O)₂]⁻ imparted increase in 1/T₂ relative to neat H₂O at pH 3 by the mole fraction of coordinated water molecules. This data was plotted against reciprocal temperature (1000/T (K⁻¹)) and fit to a four-parameter model as described previously.² The Gd—O hyperfine coupling constant, A/h, was assumed to be 3.79×10⁶ rad/s.³ Samples were prepared in neat H₂O adjusted and enriched with a small amount of H₂¹⁷O.

### Luminescence

Luminescence lifetime measurements were recorded on a Hitachi f-4500 fluorescence spectrophotometer on samples containing ˜50 mM Eu(III). Samples in D₂O were lyophilized and reconstituted three times to ensure minimization of residual protio solvent. Excitation was achieved at 396 nm and emission was recorded at 616 nm. A total of 80 replicates were acquired for each sample and the results averaged. Temporal resolution was set to 0.04 ms (0-20 ms) and the PMT voltage was set to 400 V. The luminescent lifetimes were ascertained though monoexponential fits of the data.

### Ligand Challenge

Mixtures of precisely defined concentrations of [Gd(CyPic3A)(H₂O)₂]⁻ and DTPA or MS-325-L were prepared in pH 4 citrate buffer (25 mM) and pH 7.4 Tris buffer (25 mM). Equilibration of each solution was monitored by LC-MS. Relative distributions of [Gd(CyPic3A)(H₂O)₂]⁻ and CyPic3A were determined by integrating the corresponding absorbance traces at 280 nm. MS-325 and MS-325-L were analyzed analogously at 220 nm. K_{comp} values determined between [Gd(DTPA)(H₂O)]²⁻ and various ligands were determined from conditional formation constants (K_{cond})⁴ values taken from the literature.⁶⁻¹⁶ Direct comparisons were only made between K_{cond} values determined under identical conditions.

### Kinetic Inertness

Modified conditions of Muller and co-workers were used for this experiment.^{11,12} Solutions containing 2.5 mM [Gd(CyPic3A)(H₂O)₂]⁻ and 2.5 mM Zn(OTf)₂ were combined in pH 7.0 phosphate buffer (50 mM) and placed in an incubator shaker set to 310 K. Progress of the transchelation was monitored via the decrease in 1/T₁ with time. Prior to measurement, aliquots were placed in small glass inserts and centrifuged to bring all insoluble material to the bottom so as not to interfere with the measurement.

### Synthesis of CyPic3A

The synthesis comprised the following steps:

N—BOC—N′-((6-methylpicol-2-yl)methyl)-trans-1,2-diaminocyclohexane (3). A batch of 349 mg (1.63 mmol) N—BOC-trans-1,2-diaminocyclohexane (1)¹⁴ was added to 269 mg (1.63 mmol) of methyl 6-formylpyridine-2-carboxylate (2)¹⁵ stirring in 10 mL MeOH. Small aliquots of this reaction were removed and concentrated to dryness for NMR analysis to confirm full Schiff base formation. After 2 hours, the reaction was cooled to 0° C. and 66 mg (1.75 mmoL) NaBH₄ was added in 1 mL MeOH forming a deep red solution within minutes. After 2 hours of stirring at 0° C., the reaction was quenched with satd. NaHCO_{3(aq)} and MeOH was removed via rotary evaporation. The volume of the resultant solution was doubled via addition of CH₂Cl₂ and the reaction was brought to pH 7 using 1M HCl_{(aq)}. The organic layer was separated the aqueous phase washed 3× with CH₂Cl₂. The CH₂Cl₂ was pooled, dried over Na₂SO₄ and concentrated to 502 mg (1.38 mmol, 85%) of 3 as a yellow oil. ¹H NMR (CDCl₃, 400 MHz), δ (ppm): 7.97 (d, 1H), 7.75 (t,1H), 7.54 (d, br, 1H), 5.18 (d, br, 1H, NH), 4.08 (d, 1H), 3.99-3.95 (m, 4H), 3.32 (m, br, 1H), 2.37 (m, 1H), 2.09-2.01 (m, 2H), 1.66-1.62 (m, 2H), 1.41 (s, 9H), 1.29-1.05 (m, 4H). ¹³C NMR (CDCl₃, 100 MHz), δ (ppm): 165.95, 161.82, 156.21, 147.24, 137.33, 125.70, 123.35, 79.07, 64.67, 60.42, 54.75, 51.28, 33.18, 31.97, 28.49, 24.97, 24.61. ESI⁺ (M +H⁺) m/z=364.3; calcd: 364.2.

N-((6-methyl picol-2-yl)methyl)-trans-1,2-diaminocyclohexane (4). A batch of 611 mg (1.68 mmol) 3 was dissolved in 3 mL each CH₂Cl₂: TFA. After 45 minutes, the reaction mixture was concentrated to a pink oil which was taken up in 30 mL CH₂Cl₂ and stirred over a large excess of K₂CO₃. After 3 hours, the resultant light yellow solution was filtered and concentrated to 266 mg (1.00 mmol, 60%) of 4 as a yellow oil. It should be noted that the filtrate often contained very little product when the reaction was performed on a larger scale; when this occurred, the slurry containing product and K₂CO₃ could be carried directly through to the next step (assuming 100% product) and afforded excellent yields. ¹H NMR (CDCl₃, 500 MHz), δ (ppm): 8.00 (d, 1H), 7.81 (t, 1H), 7.67 (d, 1H), 4.17 (d, 1H), 4.40-3.96 (m, 4H), 2.45 (m, 1H), 2.13 (m, 2H), 1.92-1.67 (m, 7H, NH₂ and NH found as broad resonance), 1.30-1.07 (m, 4H). ¹³C NMR (CDCl₃, 125.7 MHz), δ (ppm): 166.08, 161.88, 147.42, 137.47, 125.80, 123.55, 63.95, 55.53, 52.52, 36.14, 31.74, 35.39, 25.33. ESI⁺ (M+H⁺): m/z=264.2; calcd: 264.2.

N-((6-methylpicol-2-yl)methyl)-N,N′,N′-tri(tert-butylacetate)-trans-1,2-diaminocyclohexane (5). To 266 mg (1.13 mmol) of 4, 535 mg (3.22 mmol) of potassium iodide and 922 mg (7.13 mmol) diisopropylethylamine stirring in 3 mL DMF was added 732 mg (3.75 mmol) tert-butyl bromoacetate at RT. After 16 hours, the resultant yellow, heterogeneous solution was diluted with 50 mL CH₂Cl₂, washed with satd. K₂CO_{3(aq)}, copious water and brine. The organic portion was than dried with Na₂SO₄ and concentrated to a yellow oil. After chromatography on silica gel using 9:1 to 3:1 hexane: EtOAc, 2.600 g (4.29 mmol, 75%) of 5 was isolated as a yellow oil. ¹H NMR (CDCl₃, 500 MHz), δ (ppm): 8.05 (d, 1H), 7.92 (t, 1H), 7.76 (t, 1H), 4.12 (d, 1H), 3.94 (s, 3H), 3.80 (d, 1H), 3.45-3.20 (m, 6H), 2.62 (m, br, 1H), 2.53 (t, br, 1H). 2.07-1.99 (m, 2H), 1.66 (s, br, 2H), 1.36 (s, 27H), 1.09-1.00 (m, 4H). ¹³C NMR (CDCl₃, 125.7 MHz), δ (ppm): 171.56, 171.49, 166.05, 161.57, 146.76, 137.37, 127.89, 123.57, 80.76, 80.54, 63.18, 61.70, 58.13, 55.75, 52.83, 28.07 (C(CH₃) peaks are coincidental), 26.78, 25.81, 25.66, 18.38. ESI⁺ (M+H⁺): m/z=606.4; calcd: 606.4.

N-((6-methylpicol-2-yl)methyl)-N,N′,N′-triacetate-trans-1,2-diaminocyclohexane (CyPic3.2TFA). To a batch of 173 mg (0.29 mmol) 5 in 4 mL 1:1 THF: H₂O was added 28 mg (1.17 mmol) lithium hydroxide. After 4 hours stirring at room temperature, the reaction was concentrated to dryness and the resultant residue re-dissolved in 8 mL TFA and stirred at room temperature. After 16 hours, the reaction mixture was concentrated to dryness and purified by preparative HPLC using the method described above (general methods). After pooling of the fractions containing product followed by lyophilization, 84 mg CyPic3A•2TFA was isolated as a white powder. The NMR spectra of the isolated ligand affords broad and ill-resolved peaks when dissolved in D₂O untreated. ¹H NMR (D₂O, 500 MHz), δ (ppm): 8.53 (t, 1H), 8.31 (d, 1H), 8.14 (s, br, 1H), 4.60-3.03 (m, br, 10H), 2.28 (d, 1H), 2.13 (s, br, 1H), 1.85 (s, br, 2H), 1.51-1.26 (m, br, 4H). ¹H NMR (D₂O, pH>10, 500 MHz), δ (ppm): 7.87 (t, 1H), 7.79 (d, 1H), 7.63 (d, 1H), 3.71 (q, 2H), 3.40 (d, 1H), 3.32 (d, 1H), 2.99 (d, 1H), 2.73 (d, 1H), 2.58-2.49 (m, 2H), 2.39 (t, 1H), 2.15-2.07 (m, 2H), 1.90 (d, 1H), 1.71 (t, 2H), 1.18-1.09 (m, 3H), 0.90 (q, 1H). ¹³C NMR (D₂O, pH≧10, 125.7 MHz), δ (ppm): The ¹³C resonances were stronger and more visible at alkaline pH: 181.76, 181.44, 181.19, 174.03, 157.62, 153.67, 139.04, 127.42, 122.78, 61.44, 59.00, 58.02, 57.30, 54.23, 52.29, 25.63, 25.50, 24.72, 24.59. ESI⁺ (M+H⁺): m/z=424.2; calcd: 424.2

[Gd(CyPic3A)(H₂O)₂]⁻. To a batch of 54.5 mg (0.0837 mmol) CyPic3A•2TFA in H₂O was added 31.1 mg (0.0837 mmol) of GdCl₃.6H₂O and the solution adjusted to pH 6.5. Full chelation was affirmed by the testing of small aliquots of this solution in Arsenazo III (0.01 mM in 0.15 M pH 7 ammonium acetate buffer). ESI⁺ (MW+2H⁺): m/z=579.0; calcd: 579.1. Less than 1% free ligand was observed by LC-MS.

[Eu(CyPic3A)(H₂O)₂]⁻. To a batch of 54.8 mg (0.0841 mmol) CyPic3A•2TFA in H₂O was added 24.3 mg (0.0663 mmol) of EuCl₃.6H₂O was added and the solution adjusted to pH 7.2. Analysis by LC-MS revealed full chelation with a slight excess of ligand species present. This was done to ensure against free Eu(III) during the luminescence measurements. ESI⁺ (MW+2H⁺): m/z=574.0; calcd: 574.1

TABLE 7
Comparison of r₁ values of [Gd(CyPic3A)(H₂O)₂]⁻ to
select FDA approved Gd(III) complexes and previously
studied Gd(III) complexes of q = 2. r₁ has units of mM⁻¹s⁻¹

| | | | r₁ | r₁ | r₁ | r₁ |
|---|---|---|---|---|---|---|
| | | | 0.47 T | 1.41 T | 0.47 T | 1.41 T |
| | MW | q | 310K | 310K | 298K | 298K |
| [Gd(CyPic3A)(H₂O)₂]⁻ | 577.1 | 2 | 6.90 | 5.70 | 8.26 | 7.93 |
| [Gd(DTPA)(H₂O)]²⁻ | 564.0 | 1¹⁶ | 3.4¹⁷ | 3.26 | 4.3¹⁶ | — |
| MS-325 | 907.1 | 1¹⁸ | 5.8¹⁷ | 5.2 (1.5 T)¹⁷ | — | — |
| [Gd(HP-DO3A)(H₂O)] | 577.1 | 1¹⁶ | 3.1¹⁷ | 2.9 (1.5 T)¹⁷ | — | — |
| [Gd(DTPA-BMA)(H₂O)] | 592.1 | 1¹⁶ | 3.5¹⁷ | 3.3 (1.5 T)¹⁷ | — | — |
| [Gd(AAZTA)(H₂O)₂]⁻ | 537.1 | 2¹⁹ | — | — | 7.1¹⁹ | — |
| [Gd(HOPO)(H₂O)₂] | 790.2 | 2²⁰ | 10.5¹⁶ | — | — | — |
| [Gd(DO3A)(H₂O)₂] | 536.1 | 2¹⁷ | 4.8¹⁶ | — | — | — |
| [Gd(PCTA)(H₂O)₂] | 569.6 | 2.4²¹ | 5.42²¹ | — | 6.9²² | — |

TABLE 8
Comparison of K_{comp} of [Gd(DTPA)(H₂O)]²⁻ and MS-325 vs.
L obtained through ligand challenge (for CyPic3A) or calculation
(all other L) from thermodynamic data at pH 4 and 7.4,
and time to 80% 1/T₁ from Zn (II) challenge experiment.

| | K_{comp} | K_{comp} | | | Time |
|---|---|---|---|---|---|
| | MS-325 | MS-325 | K_{comp} | K_{comp}, | to |
| | vs. L | vs. L | [Gd(DTPA)(H₂O)]²⁻ | [Gd(DTPA)(H₂O)]²⁻ | 80% r₁ |
| | pH 4 | pH 7.4 | vs. L pH 4 | vs. L pH 7.4 | (min) |
| CyPic3A | 0.031 | 0.56 | 0.18 | .172 | 95 |
| DTPA | 0.58⁵ | 0.35⁵ | 1 | 1 | 383 |
| MS-325-L | 1 | 1 | 0.57⁵ | 2.88⁵ | 3800¹¹ |
| HP-DO3A | 0.16^{5,6} | 0.07^{5,6} | 0.22⁶ | 0.19⁶ | inert¹¹ |
| DTPA- | — | — | 0.37⁷ | .00027⁷ | 124 |
| BMA | | | | | |
| AAZTA | — | — | 0.091⁹ | .001⁹ | — |
| HOPO | — | — | 0.0079^{8,9} | 0.63^{8,9} | — |
| DO3A | 0.00046^{5,6} | 0.00017^{5,6} | 0.00048^{5,6} | 0.00050^{5,6} | inert¹¹ |
| PCTA | — | — | 0.064¹⁰ | 0.019¹⁰ | — |

### References for Example 5 Experimental Section

1. G. R. Fulmer, A. J. M. Miller, N. H. Sherden, H. E. Gottlieb, A. Nudelman, B. M. Stoltz, J. E. Bercaw and K. I. Goldberg. Organometallics, 2010, 29, 2176

2. P. Caravan, G. Parigi, J. M. Chasse, N. J. Cloutier, J. J. Ellison, R. B. Lauffer, C. Luchinat, S. A. McDermid, M. Spiller and T. J. McMurry. Inorg. Chem. 2007, 46, 6632.

3. P. Caravan, A. V. Astashkin and A. M. Raitsimring, Inorg. Chem. 2003, 42, 3972.

4. D. C. Harris, in Quantitative Chemical Analysis, 2nd edn., W.H. Freeman and Company, New York, 2003.

5. P. Caravan, C. Comuzzi, W. Crooks, T. J. McMurry, G. R. Choppin and S. R. Woulfe. Inorg. Chem. 2001, 40, 2170.

6. K. Kumar, C. A. Chang, L. C. Francesconi, D. D. Dischino, M. F. Malley, J. Z. Gougoutas and M. F. Tweedle. Inorg. Chem. 1994, 33, 3567.

7. L. Moriggi, C. Cannizzo, C. Prestinari, F. Berriere and L. Helm. Inorg. Chem. 2008, 47, 8357.

8. D. M. J. Doble, M. Melchior, B. O'Sullivan, C. Siering, J. Xu, V. C. Pierre and K. N. Raymond. Inorg. Chem. 2003, 42, 4930.

9. Z. Baranyai, F. Uggeri, G. B. Giovenzana, A. Benyei, E. Bracher and S. Aime. Chem. Eur. J. 2009, 15, 1696.

10. G. TircsO, Kovacs and A. D. Sherry. Inorg.Chem. 2006, 45, 9269.

63. S. Laurent, L. V. Elst, F. Copoix and R. N. Muller. Invest. Radiol. 2001, 36,115.

11. S. Laurent, L. V. Elst, C. Henoumon and R. N. Muller. Contrast Media Mol. Imag. 2010, 5, 305.

12. M. Polasek and P. Caravan. Inorg. Chem. 2013, 52, 4084.

13. P. D. Garimella, A. Datta, D. W. Romanini, K. N. Raymond and M. B. Francis. J. Am. Chem. Soc. 2011, 133, 14704.

14. C. Platas-Iglesias, M. Mato-Iglesias, K. Djanashvili, R. N. Muller, L. V. Elst, J. A. Peters, A. de Blas and T. Rodriguez-Blas. Chem. Eur. J. 2004, 10, 3579.

15. P. Caravan, J. J. Ellison, T. J. McMurry and R. B. Lauffer. Chem. Rev. 1999, 99, 2293.

16. M. Rohrer, H. Bauer, J. Mintorovitch, M. Requardt and H.-J. Weinmann. Invest. Radiol. 2005, 40, 715.

17. P. Caravan. Acc. Chem. Res. 2009, 42, 851.

18. S. Aime, L. Calabi, C. Cavallotti, E. Gianolio, G. B. Giovenzana, P. Losi, A. Maiocchi, G. Palmisano and M. Sisti. Inorg. Chem. 2004, 43, 7588.

19. A. Datta and K. N. Raymond, Acc. Chem. Res. 2009, 42, 938.

20. W. D. Kim, G. E. Kiefer, F. Maton, K. McMillan, R. N. Muller and A. D. Sherry. Inorg. Chem. 1995, 34, 2233

21. S. Aime, M. Botta, S. G. Crich, G. Giovenzana, R. Pagliarin, M. Sisti and E. Terreno. Magn. Reson. Chem. 1998, 36, S200.

### Example 6

Example 6 has the following experimental details: (i) DOTAlaP-derivatives: Synthesis of compounds; (ii) DOTAlaP derivatives: Relaxivity; (iii)

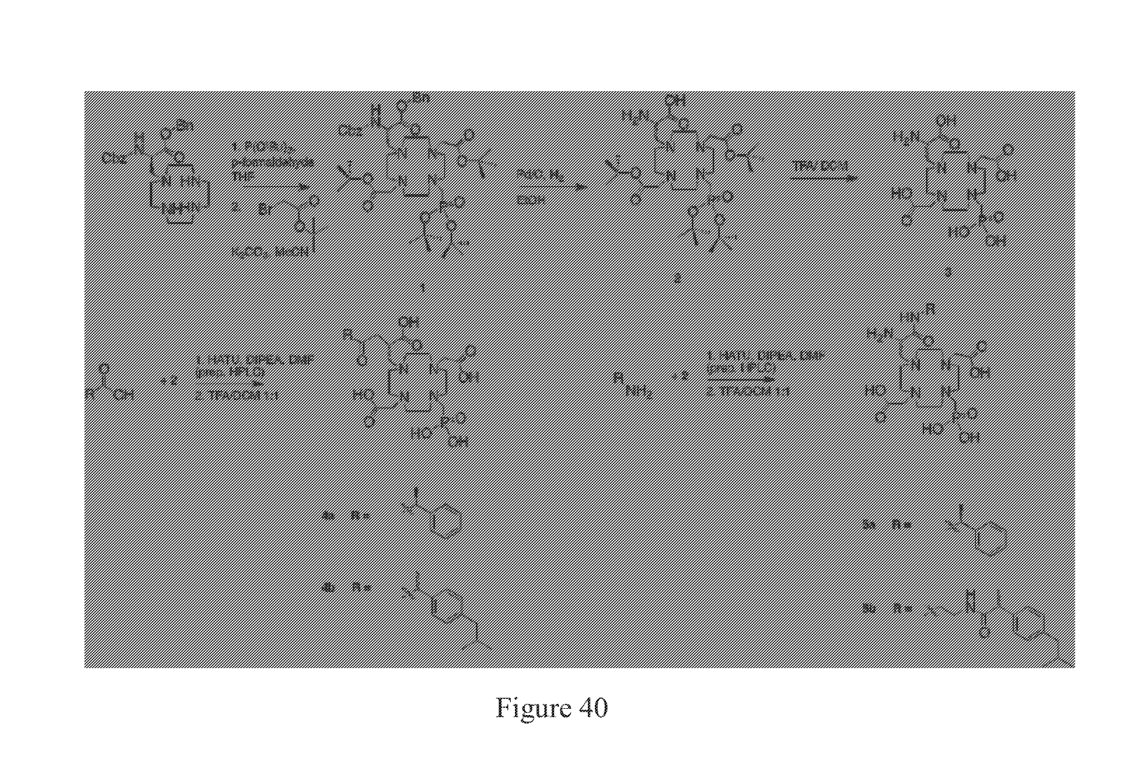

DOTAlaP derivatives: Results of Eu-luminescence lifetime derived hydration number; (iv) DOTAlaP derivatives: Results of ¹⁷O-NMR derived water exchange; (v) Modified trimer: Synthesis protocol; (vi) Animal model used for trimer/Gadovist imaging; (vii) In vivo imaging protocol: trimer and Gadovist; and (viii) In vivo imaging protocol: ‘Ibuvist’ and ‘P-Ibuvist’. FIG. 40 shows the synthesis of DOTAlaP-derivatives of Example 6.

General materials and methods. General Methods and Materials. 1H and 13C NMR spectra were recorded on a Varian 11.7 T NMR system equipped with a 5 mm broadband probe. Purification via HPLC of intermediates toward DOTAlaP derivatives was performed using method A: Injection of crude mixture onto preparative HPLC on a Rainin, Dynamax (column: 250 mm Phenomenex C18) by using A, 0.1% TFA in water; B, 0.1% TFA in MeCN, flow-rate 15 mL/min, from 5% B to 95% B over 20 minutes. HPLC purity analysis (both UV and MS detection) was carried out on an Agilent 1100 system (column: Phenomenex Luna, C18(2) 100/2 mm) with UV detection at 220, 254, and 280 nm by using method B: Injection of crude mixture onto analytical column (Phenomenex Luna, C18(2) 100/2 mm) using A, water; B, MeCN, flow-rate 0.8 mL/min, 15 minute gradient from 2% B to 60% B over 15 minutes. Monitoring of UV absorption was done at 220 nm or method C: A gradient of 95% A (10 mM ammonium acetate) to 95% B (10% 10 mM ammonium acetate/ 90% MeCN), flow-rate 0.8 mL/min, over 15 minutes. method D: A gradient of 95% A (10 mM ammonium acetate) to 35% B (10% 10 mM ammonium acetate/ 90% MeCN), flow-rate 0.8 mL/min, over 15 minutes. The synthesis of ligands was carried out as shown in FIG. 40. Chemicals were supplied by Aldrich Chemical Co., Inc., and were used without further purification. Solvents (HPLC grade) were purchased from various commercial suppliers and used as received. The monoalkylated cyclen precursor to compound 1 of FIG. 40 was synthesized as described previously (Boros et al, J. Am. Chem. Soc., 2012, 134, 19858-68). Tri-tert-butyl-phosphite was synthesized according to a procedure reported by Manning et al., Tet. lett., 2005, 46, 4707-10. General procedure for amide couplings (step to afford products 4a, 4b, 5a, 5b of FIG. 40): Amide coupling followed by deprotection of the tert-butyl protective groups was done by activation of the carboxylate with HATU (1.2 eq) and DIPEA (1.2 eq) for 5 minutes in DMF, followed by addition of the amine (1 eq) dissolved in DMF and stirring at room temperature for 18 hours. After confirmation of presence of the amide-coupling product by LCMS, the intermediate was isolated by preparative HPLC (method A), eluting between 11-13 minutes. The clean fractions containing the desired product were pooled and lyophilized to afford the intermediate as an off white powder in 15-35% yield. General deprotection procedure: Re-dissolution of intermediates described above in a 1:1 mixture of DCM and TFA, followed by stirring for 18 hours afforded the final ligand. General metal complexation procedure (with Lanthanides Gd, Eu and Tb): The ligand was dissolved in H₂O (1 mL). An amount of a stock solution containing MCl₃ 6H₂O (0.95 eq) is added to the ligand solution under monitoring of pH. The pH is adjusted to 7 using 0.1 M NaOH solution. The lightly cloudy solution is filtered and lyophilized to afford the corresponding Lanthanide complex as an off-white powder.

Compound 1. (di-tert-butyl 2,2′-(4-(3-(benzyloxy)-2-(((benzyloxy)carbonyl)amino)-3-oxopropyl)-10-((di-tert-butoxyphosphoryl)methyl)-1,4,7,10-tetraazacyclododecane-1,7-diyl)diacetate). 2-(((benzyloxy)carbonyl)amino)-3-(1,4,7,10-tetraazacyclododecan-1-yl)propanoate (1.475 g, 3.05 mmol, 1 eq) and paraformaldehyde (0.34 g, 11.45 mmol, 3.75 eq.) were dissolved in THF and stirred under N₂ for 30 minutes vigorously. Tri-tert-butyl phosphite (1.145 g, 4.5 mmol, 1.5 eq.) was then added and the reaction was stirred for 16 hours. Reaction control by LCMS showed the product as [M+H]⁺690.4. The reaction mixture was then filtered, concentrated in vacuo, and redissolved in EtOAc (200 mL). The organic layer is washed with 50 mL saturated Na₂CO₃ and 50 mL brine, dried with sodium sulfate, and concentrated to afford the di-alkylated intermediate (1.73 g, 2.5 mmol, 82%), which was used in the subsequent step without further characterization. The intermediate was dissolved in MeCN (75 mL). K₂CO₃ (0.68 g, 4.9 mmol, 2 eq.) was added and the mixture was stirred vigorously after addition of tert-butyl bromoacetate (0.766 g, 0.575 mL, 3.94 mmol, 1.6 eq). After 16 hours, water (70 mL) was added and most of the MeCN was removed in vacuo. The oily residue was taken up in EtOAc (80 mL) and washed with water and brine (100mL each). The The organic layer was separated, dried with sodium sulfate, and concentrated to afford the crude product, which was redissolved in MeCN (5 mL) and purified using preparative HPLC (method A). The product elutes at 12.2 minutes. Fractions containing the product are pooled and lyophilized to afford the product as a white powder (0.43 g, 0.46 mmol, 20% yield). ¹H-NMR (CDCl₃, 500 MHz, ppm): 9.9 (s, br, 2H), 7.36-7.34 (m, 10H), 5.25-5.16(m, 4H), 4.3(m, 1H), 3.35-2.68(m, 21H), 2.04(s, 4H), 1.51-1.43(m, 36H). ¹³C-NMR (CDCl₃, 125 MHz, ppm): 175.4, 170.6, 170.3, 135.0, 131.1, 129.4, 128.6-127.9, 81.4, 81.0, 67.7, 57.3-47.4, 30.6, 28.2, 22.1. ³¹P-NMR (CDCl₃, 200 MHz, ppm): 17.7. LC-ESI-MS: calcd. for C₄₇H₇₆N₅O₁₁P₄: 917.5 Found: 918.6 [M+H]⁺, Rₜ=7.8 min (method C).

Compound 2. 2-amino-3-(4,10-bis(2-(tert-butoxy)-2-oxoethyl)-7-((di-tert-butoxyphosphorryl)methyl)-1,4,7,10-tetraazacyclododecan-1-Apropanoic acid. Compound 1 of FIG. 40 (0.43 g, 0.47mmol) was dissolved in EtOH (40 mL). Pd/C (10% w/v, 0.215 g) was added and the reaction mixture was purged first with N₂, then charged with H₂ (1 atm). The mixture was then stirred under H₂ atmosphere for 3 hours, after which the reaction was found to be complete. The Pd/C was removed by filtration and the solvent was removed in vacuo to afford the product as a colorless oil (0.32 g, 0.46 mmol, quantitative conversion). ¹H-NMR (CD₃OD, 500 MHz, ppm): 5.15 (s, br, 1H), 4.46-2.48 (m, 24H), 1.63-1.47(m, 36H). ¹³C-NMR (CD₃OD, 125 MHz, ppm): 167.9, 160.5, 86.6, 82.6, 55.2-48.5, 30.6, 29.2, 26.9. ³¹P-NMR (CD₃OD, 200 MHz, ppm): 16.9. LC-ESI-MS: calcd. for C₃₂H₆₄N₅O₉P: 693.4 Found: 694.7 [M+H]⁺, Rₜ=5.4 min (method B).

Compound 3. 2,2′-(4-(2-amino-2-carboxyethyl)-10-(phosphonomethyl)-1,4,7,10-tetra-azacyclododecane-1,7-diyl)diacetic acid. ¹H-NMR (CD₃OD, 500 MHz, ppm): 5.15 (s, br, 2H), 4.26-2.46 (m, 25H). ¹³C-NMR (CD₃OD, 125 MHz, ppm): 175.1, 169.6, 55.4-48.9. ³¹P-NMR (CD₃OD, 200 MHz, ppm): 6.3. LC-ESI-MS: calcd. for C₁₆H₃₂N₅O₉P: 469.2 Found: 470.1 [M+H]⁺, Rₜ=1.1 min (method B).

Compound 4a. 2,2′-(4-(2-carboxy-2-(2-phenylpropanamido)ethyl)-10-(phosphonomethyl)-1,4,7,10-tetraazacyclododecane-1,7-diyl)diacetic acid. ¹H-NMR (CD₃OD, 500 MHz, ppm): 7.38-7.23 (m, 5H), 5.15 (s, br, 1H), 4.76 (m, 1H), 3.98-2.86 (m, 24H), 1.47 (t, 3H). ¹³C-NMR (CD₃OD, 125 MHz, ppm): 159.4, 140.9, 128.5-126.8, 116.7, 114.4, 53.3, 49.5, 46.1, 17.1. ³¹P-NMR (CD₃OD, 200 MHz, ppm): 0.8. LC-ESI-MS: calcd. for C₂₅H₄₀N₅O₁₀P: 601.2 Found: 602.4 [M+H]⁺, Rₜ=2.1 min (method B).

Compound 4b. 2,2′-(4-(2-carboxy-2-((1-(4-isobutylphenyl)ethyl)amino)ethyl)-10-(phosphonomethyl)-1,4,7,10-tetraazacyclododecane-1,7-diyl)diacetic acid. ¹H-NMR (CD₃OD, 500 MHz, ppm): 7.28 (d, 2H), 7.20 (d, 2H), 4.73 (s, br, 1H), 3.91-2.87 (m, 24H), 2.45(m, 2H), 1.84(m, 1H), 1.48-1.29(m, 5H), 0.89 (d, 6H). ¹³C-NMR (CD₃OD, 125 MHz, ppm): 172.8, 169.6, 140.5, 138.2, 129.5, 127.0, 54.7-48.2, 30.0, 21.3, 17.1. ³¹P-NMR (CD₃OD, 200 MHz, ppm): 3.5. LC-ESI-MS: calcd. for C₂₈H₄₈N₅O₉P: 629.3 Found: 630.5 [M+H]⁺, Rₜ=5.6 min (method B).

Compound 5a. 2,2′-(4-(2-carboxy-2-(2-(4-isobutylphenyl)propanamido)ethyl)-10-(phosphonomethyl)-1,4,7,10-tetraazacyclododecane-1,7-diyl)diacetic acid. ¹H-NMR (CD₃OD, 500 MHz, ppm): 7.39-7.26 (m, 5H), 5.02 (s, br, 1H), 4.15-2.46 (m, 24H), 1.64-1.46 (m, 3H). ¹³C-NMR (CD₃OD, 125 MHz, ppm): 169.7, 166.3, 160.4, 142.7, 128.5-125.8, 119.6, 117.3, 114.9, 112.7, 53.3-49.5, 27.2, 20.85. ³¹P-NMR (CD₃OD, 200 MHz, ppm): 6.1. LC-ESI-MS: calcd. for C₂₄ H₄₁ N₆O₈; P: 573.3 Found: 574.6 [M+H]⁺, Rₜ=2.3 min (method B).

Compound 5b. 2, 2′-(4-(2-amino-3-((2-(2-(4-isobutylphenyl)propanamido)ethyl)amino)-3-oxopropyl)-10-(phosphonomethyl)-1,4,7,10-tetraazacyclododecane-1,7-diyl)diacetic acid. ¹H-NMR (CD₃OD, 500 MHz, ppm): 7.24 (d, 2H), 7.10 (d, 2H), 4.21-2.44 (m, 30H), 1.94-1.29(m, 6H), 0.89 (d, 6H). ¹³C-NMR (CD₃OD, 125 MHz, ppm): 175.4, 169.8, 167.4, 140.2, 138.8, 128.9, 126.7, 54.6-48.1, 38.2, 30.1, 21.3, 17.1. ³¹P-NMR (CD₃OD, 200 MHz, ppm): 6.1. LC-ESI-MS: calcd. for O₃₁l H₅₄N₇O₉P: 699.3 Found: 700.6 [M+H]⁺, Rₜ=6.8 min (method B).

Lanthanide complexes. MS (ESI+): m/z [Gd(3)] calcd. for C₁₆H₂₈GdN₅O₉P: 623.1 Found 625.1 [M+2H]⁺. [Eu(3)] C₁₆H₂₈EuN₅O₉P calcd. 618.1 Found 620.1 [M+2H]⁺. [Gd(4a)] calcd. for C₂₄H₃₆GdN₅O₉P: 755.1 Found 757.2 [M+2H]⁺. [Eu(4a)] C₂₄H₃₆EuN₅O₉P calcd. 750.1 Found 752.1 [M+2H]⁺. [Gd(4b)] calcd. for C₂₉H₄₄GdN₅O₁₀P: 811.2 Found 813.4 [M+2H]⁺. [Eu(4b)] C₂₉H₄₄EuN₅O₁₀P calcd. 806.2 Found 808.4 [M+2H]⁺. [Tb(4b)] C₂₉H₄₄TbN₅O₁₀P calcd. 812.2 Found 814.4 [M+2H]⁺. [Gd(5a)] calcd. for C₂₄H₃₈GdN₆O₈P: 727.2 Found 728.2 [M+H]⁺. [Eu(5a)] C₂₄H₃₈EuN₆O₈P calcd. 722.2 Found 723.3 [M+H]⁺. [Gd(5b)] calcd. for C₃₁H₅₁GdN₇O₉P: 852.3 Found 853.5 [M+H]⁺. [Eu(5b)] C₃₁H₅₁EuN₇O₉P calcd. 849.3 Found 850.4 [M+H]⁺.

Relaxivity. Longitudinal relaxation times T1, were measured on Bruker Minispecs mq20 (20 MHz) and mq60 (60 MHz) using an inversion recovery method with 10 inversion time values ranging from 0.05×T1 to 5×Ti. Relaxivity was calculated from a linear plot of 3 different concentrations ranging from 0.25 to 1.0 mM versus the corresponding inverse relaxation times. The temperature was adjusted to 37° C. See Table 9.

TABLE 9

| Compound | Gd(3) | Gd(4a) | Gd(4b) | Gd(5a) | Gd(5b) |
|---|---|---|---|---|---|
| r₁ (20 MHz, 37° C., | 6.4 | 3.9 | 4 | 5.4 | 3.2 |
| mM⁻¹s⁻¹) | | | | | |
| r₁ (60 MHz, 37° C., | 6.1 | 3.3 | 3.8 | 4.1 | 3.0 |
| mM⁻¹s⁻¹) | | | | | |

### Determination of q by Luminescence Lifetime Measurements

Luminescence lifetime measurements of Eu complexes in H₂O and D₂O were performed on a Hitachi f-4500 fluorescence spectrophotometer. Concentrations of the samples were 5-10 mM. For the measurements in D₂O, the complexes were first dissolved in D₂O (99.98% D), lyophilized, and dissolved in D₂O again to reduce the amount of residual H₂O. Measurements were taken with the following settings: Excitation at 396 nm, emission at 616 nm, 30 replicates, 0.04 ms temporal resolution (0-20 ms), PMT voltage=400 V. Lifetimes were obtained from monoexponential fits of the data using Igor Pro software. See Table 10

TABLE 10

| Compound | Eu(3) | Eu(4a) | Eu(4b) | Tb(4b) | Eu(5a) |
|---|---|---|---|---|---|
| 1/T (H₂O) | 0.55 | 0.51 | 0.59 | 0.33 | 0.52 |
| 1/T (D₂O) | 2.14 | 1.31 | 1.77 | 0.42 | 1.76 |
| q | 1.4 | 0.5 | 1.1 | 0.15 | 1 |

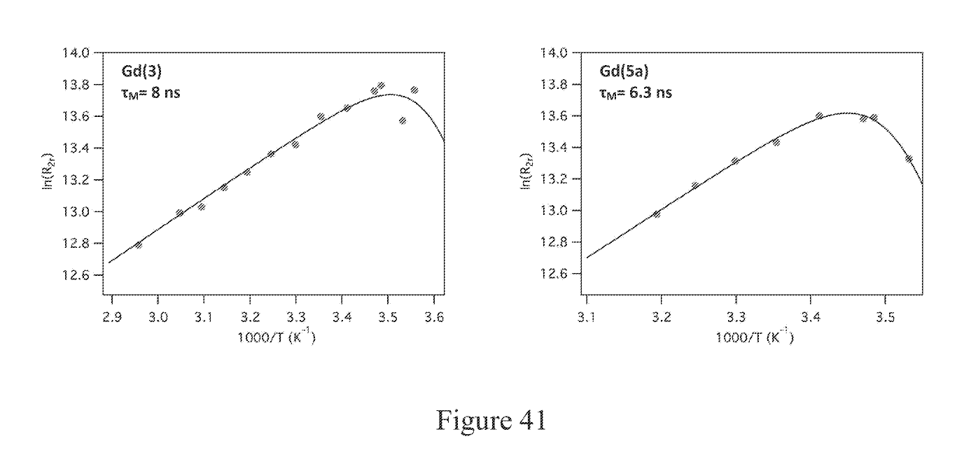

¹⁷O NMR of Gd complexes for Determination of T_{M}. ¹⁷O NMR measurements of solutions were performed at 11.7 T on 350 μL samples contained in 5 mm standard NMR tubes on a Varian spectrometer. Temperature was regulated by air flow controlled by a Varian VT unit. ¹⁷O transverse relaxation times of a >4 mM solution of Gd(3) and Gd(5a) (pH 7.4, 10 mM PBS buffer) were measured using a CPMG sequence. The concentration of the sample was determined by ICP-MS. Reduced relaxation rates, 1/T₂ᵣ were calculated from the difference of 1/T₂ between the sample and the water blank, and then divided by the mole fraction of coordinated water. The temperature dependence of 1/T₂ᵣ was fit to a 4-parameter model as previously described. The Gd—O hyperfine-coupling constant, A/h, was assumed to be 3.8×10⁶ rad/s, the Gd—O distance was assumed to be 3.1 Å. See FIG. 41 and Table 11.

TABLE 11

| | Gd(3) | Gd(5a) |
|---|---|---|
| ³¹⁰kₑₓ × 10⁶ (s⁻¹) | 125 ± 4 | 158 ± 3 |
| ΔH^{‡} (kJ mol⁻¹) | 13.5 ± 1.12 | 22.98 ± 1.76 |
| ³¹⁰ T_{M} (ns) | 8.1 ± 0.25 | 6.36 ± 0.09 |

Synthesis of modified trimer Gd₃L3-COOH.: HOOC—(CH₂)CONH—Gd(DOTAla)-G-Gd(DOTAla)-G-Gd(DOTAla)-GPC(Acm)-CONH₂. Manual solid phase peptide synthesis was carried out as previously reported (Boros et al, J. Am. Chem. Soc., 2012, 134, 19858-68). The originally synthesized sequence for H₉L3 was altered to HOOC—(CH₂)CONH-DOTAla-G-DOTAla-G-DOTAla-GPC(Acm)-CONH₂ by following the last DOTAla coupling step with N-terminal capping with succinic anhydride (10 eq succinic anhydride, 10 eq DIPEA, 2 hours). The peptide was then subsequently cleaved from the solid support using the cleavage-cocktail (TFA/DDT/TIPS/Water (9.25:0.25:0.25:0.25)). The resin was filtered off and the filtrate concentrated with a gentle nitrogen flow, and resuspended in cold Et₂O. The crude peptide was isolated, lyophilized and complexed with Gd. The trimeric metal complex was then purified using preparative HPLC (column: 250 mm Phenomenex C18, A, 10mM ammonium acetate in water; B, 10% 10 mM ammonium acetate in water with 90% MeCN, flow-rate 15 mL/min, from 5% B to 35% B over 15 minutes, HPLC, method D: Rₜ=6.6 min, MS-ESI: m/z: 1160.3 (calcd. 1160.5) [M+2H]²⁺.

Animal model for imaging with Gadovist/ Gd₃L3-COOH: All experiments were performed in accordance with the NIH Guide for the Care and Use of Laboratory Animals. Female nude mice (nu/nu), 4-6 weeks of age, were used in this study. For intracranial implantation, 10⁵ human U87 cells suspended in 5 μL of sterile PBS were injected into the right frontal hemisphere of all the animals using a stereotactic fixation device. Tumor growth was staged every 5 days using T₂ weighted images. After 17 days, the tumors were found to have reached a suitable size for imaging and quantification.

Imaging protocol Gadovist/ Gd₃L3-COOH: Animals were anesthetized with isoflurane (1-2%) and placed in a specially designed cradle with body temperature maintained at 37° C. The tail vein was cannulated for intravenous (i.v.) delivery of the contrast agent while the animal was positioned in the scanner. Imaging was performed at 4.7 T, using a custom-built transmit-receive surface coil to acquire Dynamic Contrast enhanced (DCE) MRI images and T1-weighted images. Doses of the contrast agents were adjusted to 150 μmol/kg (Gadovist/ Gd₃L3-COOH, on a per Gd basis). T1-weighted images were acquired using a Fast Low Angle Shot (FLASH) sequence. T1 and T2 weighted images were acquired before and after the DCE image acquisition. Image acquisition parameters were: TE/TR=2.5/83 ms, averages=16, field-of-view=2.0 cm, image matrix=144×144 (in-plane resolution=139 m), 0.5 mm slice thickness, 9 image slices. The DCE sequence consisted of a T1-weighted gradient-echo sequence with TE=2.1 ms, TR=25 ms, 2 averages, flip angle=60°, FOV=2.0 cm, matrix=72×72 (in-plane resolution=278 μm), 0.5 mm slice thickness, 1 image slice, 90 repetitions, temporal resolution=3.6 s. The contrast agent was injected approximately 1 minute after commencement of the DCE imaging sequence using an intravenous tail vein catheter. The signal intensity in the tumor region of interest (ROI) was analyzed using an in-house written MATLAB program, which models the tumor signal enhancement using the two-compartment model (Tofts et al, Magn. Reson. Med., 1991 17, 357-367; Tofts et al., J. Magn. Reson. Imaging, 1997, 7, 91-101, 3; Tofts et al., J Magn. Reson. Imaging 1999, 10: 223-232), to extract the volume fraction of the extra-vascular extra-cellular (EES) space (vₑ), the volume transfer constant between the plasma and EES (K^{trans}), and the rate constant between the EES and the blood plasma (kₑₚ). Briefly, the time dependence of the tumor signal intensity is fit to equation 1.

$$S ( t ) = {M}_{0} \frac{( 1 - {e}^{- TR ⋆ R 1 ( t )} ) ⋆ sin ( α )}{1 - cos ( α ) ⋆ {e}^{- TR ⋆ R 1 ( t )}} \qquad [ Eq . 1 ]$$

where R1(t) is the longitudinal relaxation rate, a is the flip angle, and TR is the repetition time. R1(t) depends on the contrast agent relaxivity (r₁), the pre-contrast longitudinal relaxation rate (R1(0)), and the tissue concentration of the contrast agent tracer (Cₜ(t)) as described by equation 2.

R1(t)=R1(0)+r1*Cₜ(t) [Eq. 2]

In turn, Cₜ(t) is derived from the arterial input function (AIF), Cₚ(t), as described by equation 3.

$${C}_{t} ( t ) = {K}^{trans} \cdot D \cdot [ \frac{{a}_{1} \cdot ( {e}^{- {k}_{ep} \cdot t} - {e}^{- {k}_{1} \cdot t} )}{( {k}_{1} - {k}_{ep} )} + \frac{{a}_{2} \cdot ( {e}^{- {k}_{ep} \cdot t} - {e}^{- {k}_{2} \cdot t} )}{( {k}_{2} - {k}_{ep} )} ] \\ {C}_{p} ( t ) = D \cdot [ {a}_{1} \cdot {e}^{- {k}_{1} \cdot t} + {a}_{2} \cdot {e}^{- {k}_{2} \cdot t} ] \qquad [ Eq . 3 ]$$

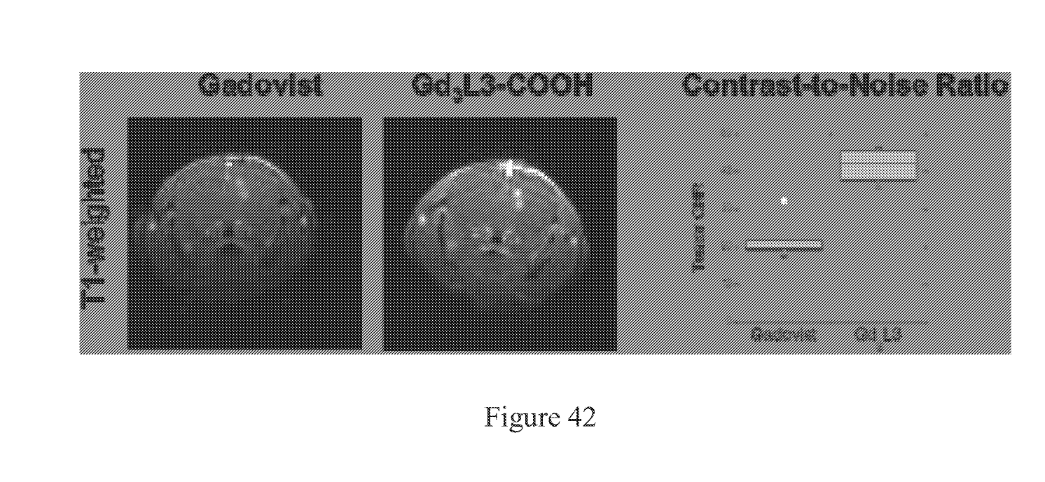

The AIF is modeled as a bi-exponential function with parameters a₁ and k₁ describing the fast equilibration between the plasma and extracellular space, a₂ and k₂ describing the clearance of contrast agent by the kidneys, and D is the contrast agent dose (mmol Gd/kg bodyweight) administered by intravenous injection. We have used the AIF parameters determined empirically (McGrath et al., Magn. Reson. Med., 2009, 61, 1173-1184). See FIG. 42

Probe preparation MS-325/Gd(9)/Gd(4a): Gd(9) and Gd(4a) were prepared according to the procedure of Example 3 as described above. MS-325 was obtained from a commercial source as a 0.1M solution (Gadofosveset, trade names Vasovist, Ablavar). All stock solutions were diluted with PBS to obtain a 40 mM probe concentration, which was determined by ICP-MS.

Animal model for blood pool imaging with MS-325/Gd(9)/Gd(4a): All experiments were performed in accordance with the NIH Guide for the Care and Use of Laboratory Animals. Healthy female nude mice (nu/nu), 4-6 weeks of age were used in this study.

Animal model for liver imaging with Gd(9): Strain A/J male mice (Jackson Laboratories, Bar Harbor, Me., USA) were administered 0.1 ml of a 40% solution of carbon tetrachloride (CCl₄; Sigma) in olive oil by oral gavage, three times a week for 20 weeks (n=8); age matched controls received only pure olive oil (n=4), or no vehicle (n=6). Animals from both models were imaged one week after the last injection to avoid acute effects of DEN or CCl₄.

Imaging protocol MS-325/Gd(9)/Gd(4a): Animals were anesthetized with isoflurane (1-2%) and placed in a specially designed cradle with body temperature maintained at 37° C. The tail vein was cannulated for intravenous (i.v.) delivery of the contrast agent while the animal was positioned in the scanner. Imaging was performed at 4.7 T, using a custom-built volume coil to acquire T1-weighted images. The animal was positioned such that the major organs (heart, stomach, liver, intestines, kidney) were visible in the field of view. Doses of the contrast agents were adjusted to 100 μmol/kg. T1-weighted images were acquired using a 3D Fast Low Angle Shot (FLASH) sequence. MS-325/Gd(4a): FA: 40. Resolution: 0.25 mm/pxl isotropic, Averages: 1, FOV: 4.8 cm×2.4 cm×2.4 cm, Matrix Size: 192×96×96, TE: 1.54, TR: 15.29), with one gated and one non-gated image acquired pre-injection, followed by 5 non-gated 3D FLASH scans, which then were followed by 7 gated and 7 non-gated 3D flash scans up to 1h post injection. Gd(9) healthy animals: FA: 20. Resolution: 0.375mm/pxl isotropic, Averages: 1, FOV: 4.8 cm×2.4 cm×2.4 cm, Matrix Size: 128×64×64, TE: 1.35, TR: 11.01. Gd(9) CCl₄ mice: FA: 20. Resolution: 0.25 mm/pxl isotropic, Averages: 1, FOV: 4.8 cm×2.4 cm×2.4 cm, Matrix Size: 192×96×96, TE: 1.54, TR: 15.29.

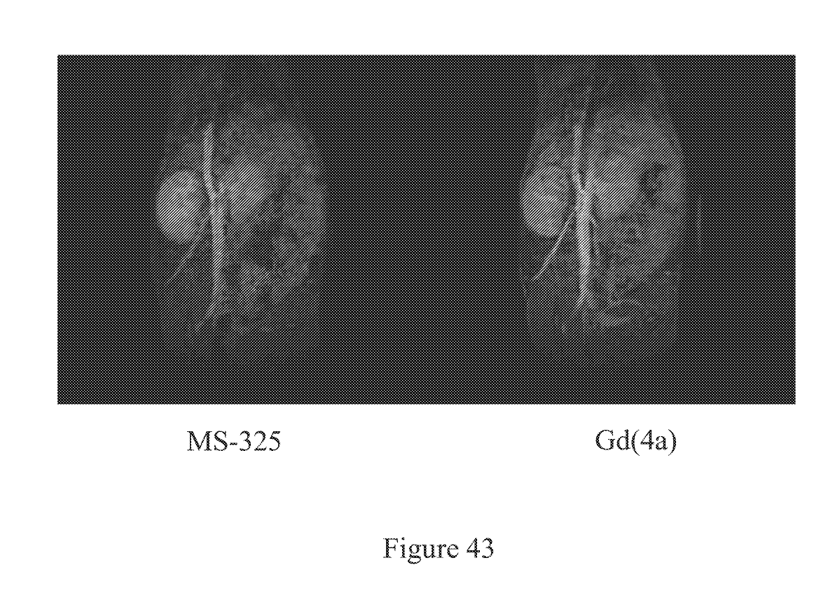

FIG. 43 shows 1 minute post injection images obtained with MS-325 (left) and Gd(4a). Gd(4a) of Example 6 shows visibly better contrast in the vena cava, which can be quantified as 38±2% better contrast (vs. muscle). The same dose of agent was used for both scans.

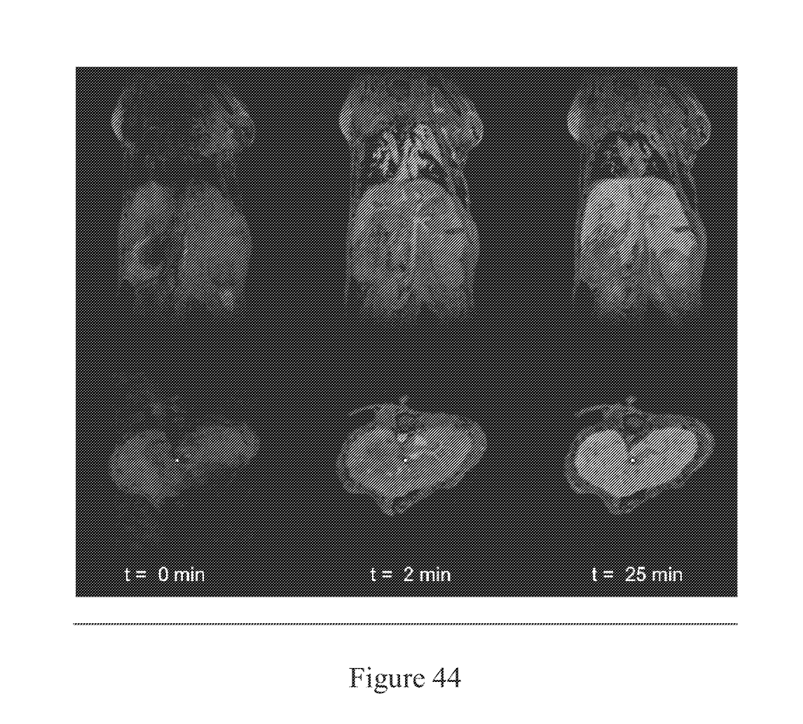

FIG. 44 shows from far left to right: A schematic overview of imaged area on a mouse, coronal slices are shown on top, axial slices on bottom. Pre-injection T₁ weighted scan (t=0 minutes) followed by continuous acquisition of T₁ weighted scans (t=2 minutes, 25 minutes) with same parameters as the pre-injection scan. Early time point (2 minutes) shows enhancement of vasculature. The late time point (25 minutes) shows enhancement of hepatic tissue while the agent has entirely cleared from the blood pool.

Although the present invention has been described in detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

1. A compound for diagnosing or treating a subject, the compound having the formula (I): 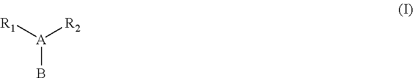
wherein A is a first amino acid residue,
wherein B is a chelate complex comprising a chelator and a metal ion, the chelator comprising a ring of atoms, the chelator forming at least one coordinate bond with the metal ion,
wherein the first amino acid residue is bonded to an atom of the ring of the chelator,
wherein the first amino acid residue has a carbonyl group oxygen that forms a coordinate bond with the metal ion,
wherein R₁ is a moiety comprising hydrogen, or an amino acid residue, or combinations thereof,
wherein R₂ is a moiety comprising hydrogen, or an amino acid residue, or combinations thereof, and
wherein at least one of R₁ and R₂ comprises an amino acid residue.

2. The compound of claim 1 wherein the compound has the formula (II): 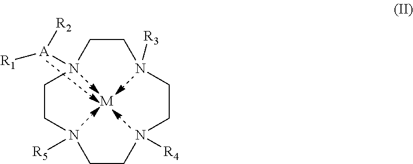
wherein A, R₁, and R₂ are as defined above, and at least one of R₁ and R₂ comprises an amino acid residue,
wherein R₃, R₄, and R₅ are independently selected from the group consisting of H, CH₂CO₂H, CH₂CH₂CO₂H, CH₂C(O)NR⁶R⁷, CH₂NHCOR⁶, CH₂C(O)N(OH)R⁶, CH₂C(O)NHSO₂R⁶, CH₂NHSO₂R⁶, CH₂N(OH)C(O)R⁶, CH₂P(R⁶)O₂R⁷, CH₂PO₃R⁶R⁷, wherein R⁶ and R⁷ are independently selected from the group consisting of H, CO₂H, C₁-C₆ alkyl, C₁₋₆CO₂H, CH(CO₂H)C₁₋₆CO₂H, C₁₋₆CF₃, C₁₋₆CCl₃, C₁₋₆CBr₃, C₁₋₆Cl₃, or C₁₋₆PO₃R⁹R¹⁰, wherein R⁹ and R¹⁹ are independently selected from the group consisting of H, CO₂H, C₁-C₆ alkyl, C₁₋₆CO₂H, CH(CO₂H)C₁₋₆CO₂H;
wherein M is the metal ion, and
wherein an atom of at least one of R₃, R₄, and R₅ in the compound forms a coordinate bond with the metal ion.

3. The compound of claim 1 wherein:
the first amino acid residue is selected from residues of alanine, cysteine, aspartic acid, glutamic acid, phenylalanine, glycine, histidine, isoleucine, lysine, leucine, methionine, asparagine, pyrrolysine, proline, glutamine, arginine, serine, threonine, selenocysteine, valine, tryptophan, and tyrosine.

4. The compound of claim 1 or claim 2 wherein:
the first amino acid residue is an alanine residue.

5. The compound of claim 1 wherein:
the metal ion is selected from ions of gadolinium, europium, terbium, manganese, iron, ⁴⁵Ti, ⁵¹Mn, ⁵²Mn, ^{52m}Mn, ⁵²Fe, ⁶⁰Cu, ⁶¹Cu, ⁶²Cu, ⁶⁴Cu, ⁶⁷Cu, ⁶⁷Ga, ⁶⁸Ga, ⁷²As, ⁸⁶Y, ⁸⁹Zr, ⁹⁰Nb, ^{94m}Tc, ^{99m}Tc, ¹¹⁰In, ¹¹¹In, ¹¹³In, ¹⁷⁷Lu, ²⁰¹Tl, ²¹²Pb ²¹³Bi, or 225Ac.

6. The compound of claim 1 wherein:
the metal ion is paramagnetic.

7. The compound of claim 1 wherein:
the metal ion is selected from paramagnetic metal ions having atomic numbers 21-29, 43, 44, and 57-83.

8. The compound of claim 1 wherein:
A has limited rotational freedom.

9. The compound of claim 1 wherein:
R₁ includes a cysteine residue, and
R₂ includes a cysteine residue.

10. The compound of claim 1 wherein:
R₁ includes a cysteine residue, and
R₂ includes a cysteine residue, and
R₁ and R₂ are linked by a disulfide bond.

11. The compound of claim 1 wherein:
at least one of R₁ and R₂ comprises a fluorescent moiety.

12. The compound of claim 10 wherein:
the fluorescent moiety has an absorption wavelength maxima in the range of 550 to 1000 nanometers, preferably 650 to 850 nanometers.

13. The compound of claim 12 wherein:
the fluorescent moiety is selected from cyanine dyes, carbocyanine dyes, and CyAL dyes.

14. The compound of claim 1 wherein:
the compound has a per-metal r₁ relaxivity of greater than 4 mM^{'11}s⁻¹.

15. The compound of claim 1 wherein:
the compound has a mean water residency time of 5 to 30 nanoseconds.

16. The compound of claim 1 wherein:
at least one of R₁ and R₂ comprises a blood plasma binding moiety.

17. The compound of claim 1 wherein:
at least one of R₁ and R₂ comprises a targeting moiety that can target a site in the subject.

18. The compound of claim 1 wherein:
the targeting moiety is selected from proteins, enzymes, peptides, antibodies, and drugs.

19. The compound of claim 1 wherein:
the compound is a contrast agent for magnetic resonance imaging.

20. The compound of claim 1 wherein:
the compound has the formula (III): 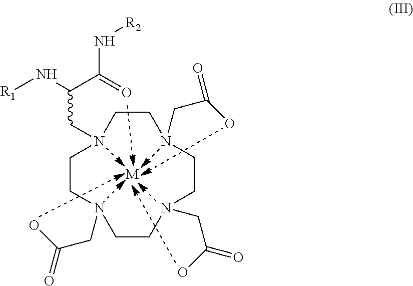
wherein R₁ and R₂ are as defined above, and at least one of R₁ and R₂ comprises an amino acid residue, and
wherein M is the metal ion.

21. The compound of claim 20 wherein:
the metal ion is Gd³⁺.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. A compound for diagnosing or treating a subject, the compound having the formula (IV): 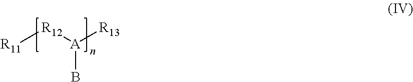
wherein A is a first amino acid residue,
wherein B is a chelate complex comprising a chelator and a metal ion, the chelator comprising a ring of atoms, the chelator forming at least one coordinate bond with the metal ion,
wherein the first amino acid residue is bonded to an atom of the ring of the chelator,
wherein the first amino acid residue has a carbonyl group oxygen that forms a coordinate bond with the metal ion,
wherein R₁₁ is a moiety comprising hydrogen, or an amino acid residue, or combinations thereof,
wherein R₁₂ is nothing or a moiety comprising hydrogen, or an amino acid residue, or combinations thereof,
wherein R₁₃ is a moiety comprising hydrogen, or an amino acid residue, or combinations thereof.
wherein at least one of R₁₁ and R₁₃ comprises an amino acid residue, and
wherein n is an integer of 2 or more.

35. The compound of claim 34 wherein: 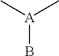
has the formula (V) 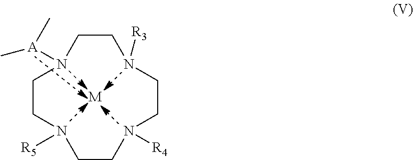
wherein R₃, R₄, and R₅ are independently selected from the group consisting of H, CH₂CO₂H, CH₂CH₂CO₂H, CH₂C(O)NR⁶R⁷, CH₂NHCOR⁶, CH₂C(O)N(OH)R⁶, CH₂C(O)NHSO₂R⁶, CH₂NHSO₂R⁶, CH₂N(OH)C(O)R⁶, CH₂P(R⁶)O₂R⁷, CH₂PO₃R⁶R⁷, wherein R⁶ and R⁷ are independently selected from the group consisting of H, CO₂H, C₁-C₆ alkyl, C₁₋₆CO₂H, CH(CO₂H)C₁₋₆CO₂H, C₁₋₆CF₃, C₁₋₆CCl₃, C₁₋₆CBr₃, C₁₋₆Cl₃, or C₁₋₆PO₃R⁹R¹⁰, wherein R⁹ and R¹⁰ are independently selected from the group consisting of H, CO₂H, C₁-C₆ alkyl, C₁₋₆CO₂H, CH(CO₂H)C₁₋₆CO₂H;
wherein M is the metal ion, and
wherein an atom of at least one of R₃, R₄, and R₅ in the compound forms a coordinate bond with the metal ion.

36. The compound of claim 34 wherein: 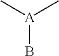
has the formula (VI) 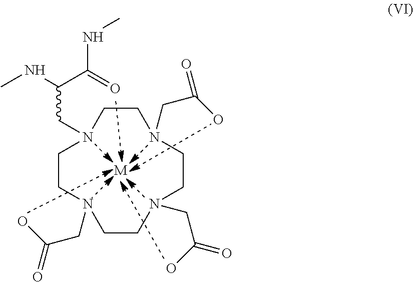
wherein M is the metal ion.

37. The compound of claim 34 wherein:
the first amino acid residue is selected from residues of alanine, cysteine, aspartic acid, glutamic acid, phenylalanine, glycine, histidine, isoleucine, lysine, leucine, methionine, asparagine, pyrrolysine, proline, glutamine, arginine, serine, threonine, selenocysteine, valine, tryptophan, and tyrosine.

38. The compound of claim 34 wherein:
the first amino acid residue is an alanine residue.

39. The compound of claim 34 wherein:
the metal ion is selected from ions of gadolinium, europium, terbium, manganese, iron,⁴⁵Ti, ⁵¹Mn, ⁵²Mn, ^{52m}Mn ⁵²Fe, ⁶⁰Cu, ⁶¹Cu, ⁶²Cu, ⁶⁴Cu, ⁶⁷Cu, ⁶⁷Ga, ⁶⁸Ga, ⁷²_{As,} ⁸⁶Y, ⁸⁹Zr, ⁹⁰Nb, ^{94m}Tc, ^{99m}Tc, ¹¹⁰In, ¹¹¹In, ¹¹³In, ¹⁷⁷Lu, ²⁰¹Tl, ²¹²Pb ²¹³Bi, or ²²⁵Ac.

40. The compound of claim 34 wherein:
the metal ion is an ion of gadolinium.

41. The compound of claim 34 wherein::
A has limited rotational freedom.

42. The compound of claim 34 wherein:
R₁₁ includes a cysteine residue, and
R₁₃ includes a cysteine residue.

43. The compound of claim 34 wherein:
R₁₁ includes a cysteine residue, and
R₁₃ includes a cysteine residue, and
R₁₁ and R₁₃ are linked by a disulfide bond.

44. The compound of claim 34 wherein:
at least one of R₁₁ and R₁₃ comprises a fluorescent moiety.

45. The compound of claim 44 wherein:
the fluorescent moiety is bonded to a phenylalanine residue.

46. The compound of claim 34 wherein:
the compound has a per-metal r₁ relaxivity of greater than 4 mM⁻¹ s^{'1}.

47. The compound of claim 34 wherein:
the compound has a mean water residency time of 5 to 30 nanoseconds.

48. The compound of claim 34 wherein:
at least one of R₁₁ and R₁₃ comprises an albumin binding moiety.

49. The compound of claim 34 wherein:
at least one of R₁₁ and R₁₃ comprises a targeting moiety that can target a site in the subject.

50. The compound of claim 49 wherein:
the targeting moiety is selected from proteins, enzymes, peptides, antibodies, and drugs.

51. The compound of claim 34 wherein:
the compound is a contrast agent for magnetic resonance imaging.

52. A compound having the formula (VIII): 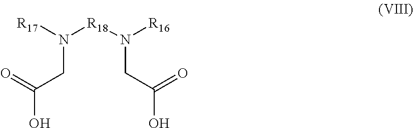
wherein R₁₆ is selected from substituted or unsubstituted alkyl carboxylates, substituted or unsubstituted cycloalkyl carboxylates, substituted or unsubstituted heterocyclic carboxylates, and amino acids,
wherein R₁₇ is selected from substituted or unsubstituted alkyl carboxylates, substituted or unsubstituted cycloalkyl carboxylates, substituted or unsubstituted heterocyclic carboxylates, and amino acids, and
wherein R₁₈ is selected from substituted or unsubstituted alkylenes and substituted or unsubstituted cycloalkylenes.

53. The compound of claim 52, wherein R₁₈ is selected from unsubstituted cycloalkylenes.

54. The compound of claim 52, wherein R₁₈ is cyclohexylene.

55. The compound of claim 52, wherein R₁₆ is selected from unsubstituted alkyl carboxylates.

56. The compound of claim 52, wherein R₁₆ is C₁-C₂₀ alkyl carboxylate.

57. The compound of claim 52, wherein R₁₆ is methyl carboxylate.

58. The compound of claim 52, wherein R₁₇ is carboxyalkylpyridine.

59. The compound of claim 52, wherein R₁₇ is carboxy-(C₁-C₂₀)alkyl-pyridine.

60. The compound of claim 52, wherein R₁₇ is carboxymethylpyridine.

61. The compound of claim 52, wherein:
R₁₆ is methyl carboxylate,
R₁₇ is carboxymethylpyridine, and
R₁₈ is cyclohexylene.

62. A compound having the formula (IX): 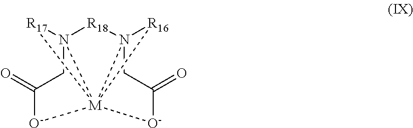
wherein R₁₆ is selected from substituted or unsubstituted alkyl carboxylates, substituted or unsubstituted cycloalkyl carboxylates, substituted or unsubstituted heterocyclic carboxylates, and amino acids,
wherein R₁₇ is selected from substituted or unsubstituted alkyl carboxylates, substituted or unsubstituted cycloalkyl carboxylates, substituted or unsubstituted heterocyclic carboxylates, and amino acids,
wherein R₁₈ is selected from substituted or unsubstituted alkylenes and substituted or unsubstituted cycloalkylenes, and
wherein M is a metal ion.

63. The compound of claim 62, wherein R₁₈ is selected from unsubstituted cycloalkylenes.

64. The compound of claim 62, wherein R₁₈ is cyclohexylene.

65. The compound of claim 62, wherein R₁₆ is selected from unsubstituted alkyl carboxylates.

66. The compound of claim 62, wherein R₁₆ is C₁-C₂₀ alkyl carboxylate.

67. The compound of claim 62, wherein R₁₆ is methyl carboxylate.

68. The compound of claim 62, wherein R₁₇ is carboxyalkylpyridine.

69. The compound of claim 62, wherein R₁₇ is carboxy-(C₁-C₂₀)alkyl-pyridine.

70. The compound of claim 62, wherein R₁₇ is carboxymethylpyridine.

71. The compound of claim 62, wherein:
R₁₆ is methyl carboxylate,
R₁₇ is carboxymethylpyridine, and
R^{y} is cyclohexylene.

72. The compound of claim 62, wherein:
the metal ion is selected from ions of gadolinium, europium, terbium, manganese, iron, ⁴⁵Ti, ⁵¹Mn, ⁵²Mn, ^{52m}Mn, ⁵²Fe, ⁶⁴Cu, ⁶⁷Cu, ⁶⁷Ga, ⁶⁸Ga, ⁷²As, ⁸⁶Y, ⁸⁹Zr, ⁹⁰Nb, ^{94m}Tc, ^{99m}Tc, ¹¹⁰In, ¹¹¹In, ¹¹³In, ¹⁷⁷Lu, ²⁰¹In, ²¹²Pb ²¹³Bi, or ²²⁵Ac.

73. The compound of claim 62 wherein:
the metal ion is paramagnetic.

74. The compound of claim 62 wherein:
the metal ion is selected from paramagnetic metal ions having atomic numbers 21-29, 43, 44, and 57-83.

75. The compound of claim 62 wherein:
the compound has a per-metal r₁ relaxivity of greater than 4 mM⁻¹ s⁻¹.

76. The compound of claim 62 wherein:
the compound has a mean water residency time of 5 to 30 nanoseconds.

77. The compound of claim 62 wherein:
the compound is a contrast agent for magnetic resonance imaging.

78. The compound of claim 62 wherein:
the metal ion is Gd³⁺.

79. The compound of claim 62 wherein the compound is heptadentate.

80. The compound of claim 62, wherein the metal ion coordinates with two molecules of water.

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. A compound for diagnosing or treating a subject, the compound having the formula (X): 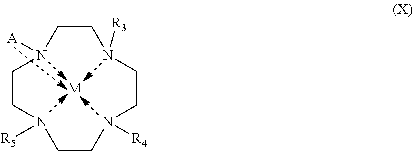
wherein A is selected from 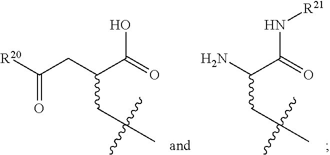
wherein R₃, R₄, and R₅ are independently selected from the group consisting of H, CH₂CO₂H, CH₂CH₂CO₂H, CH₂C(O)NR⁶R⁷, CH₂NHCOR⁶, CH₂C(O)N(OH)R⁶, CH₂C(O)NHSO₂R⁶, CH₂NHSO₂R⁶, CH₂N(OH)C(O)R⁶, CH₂P(R⁶)O₂R⁷, CH₂PO₃R⁶R⁷, wherein R⁶ and R⁷ are independently selected from the group consisting of H, CO₂H, C₁-C₆ alkyl, C₁₋₆CO₂H, CH(CO₂H)C₁₋₆CO₂H, C₁₋₆CF₃, C₁₋₆CCl₃, C₁₋₆CBr₃, C₁₋₆Cl₃, or C₁₋₆PO₃R⁹R¹⁰, wherein R⁹ and R¹⁰ are independently selected from the group consisting of H, CO₂H, C₁-C₆ alkyl, C₁₋₆CO₂H, CH(CO₂H)C₁₋₆CO₂H;
wherein R²⁰ and R²¹ are independently selected from 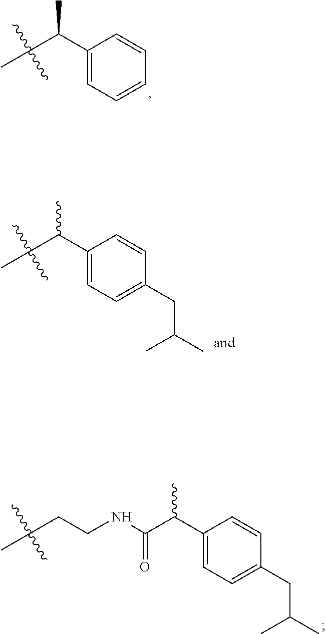
and
wherein M a metal ion.

101. The compound of claim 100 wherein:
the metal ion is selected from ions of gadolinium, europium, terbium, manganese, iron, ⁴⁵Ti, ⁵¹Mn, ⁵²Mn, ^{52m}Mn ⁵²Fe, ⁶⁰Cu, ⁶¹Cu, ⁶²Cu, ⁶⁴Cu, ⁶⁷Cu, ⁶⁷Ga, ⁶⁸Ga, ⁷²As, ⁸⁶Y, ⁸⁹Zr, ⁹⁰Nb, ^{94m}Tc, ^{99m}Tc, ¹¹⁰In, ¹¹¹In, ¹¹³In, ¹⁷⁷Lu, ²⁰¹Tl, ²¹²Pb ²¹³Bi, or ²²⁵Ac.

102. The compound of claim 100 wherein the metal ion is paramagnetic.

103. The compound of claim 100 wherein the metal ion is selected from paramagnetic metal ions having atomic numbers 21-29, 43, 44, and 57-83.

104. The compound of claim 100 wherein:
the compound has a per-metal r₁ relaxivity of greater than 3 mM⁻¹ s⁻¹.

105. The compound of claim 100 wherein:
the compound has a mean water residency time of 5 to 30 nanoseconds.

106. The compound of claim 100 wherein:
the compound is a contrast agent for magnetic resonance imaging.

107. The compound of claim 100 wherein:
the metal ion is Gd³⁺.

108. The compound of claim 100 wherein:
R₃ is CH₂CO₂H,
R₄ is CH₂PO₃R⁶R⁷, and
R₅ is CH₂CO₂H.

109. The compound of claim 108 wherein:
R⁶ and R⁷ are H.

110. (canceled)

111. (canceled)

112. (canceled)

113. (canceled)

114. (canceled)

115. (canceled)

116. (canceled)
